(19) United States
(12) Patent Application Publication
(10) Pub. No.: US 2024/0238394 A1
(43) Pub. Date: Jul. 18, 2024

(54) COMPOSITIONS, SYSTEMS AND METHODS OF RNA EDITING USING DKC1

(71) Applicant: Modit Therapeutics Beijing Limited, Beijing (CN)

(72) Inventors: Meifang ZHANG, Beijing (CN); Chengqi YI, Beijing (CN)

(21) Appl. No.: 18/563,822

(22) PCT Filed: May 26, 2022
(86) PCT No.: PCT/CN2022/095172

§ 371 Date: Nov. 22, 2023

(30) Foreign Application Priority Data
May 26, 2021 (WO) PCT/CN2021/096122

(51) Int. Cl. A61K 38/52; A61K 31/7105; A61K 48/00; C12N 9/90; C12N 15/11

(52) U.S. Cl. CPC A61K 38/52; A61K 31/7105; A61K 48/005; C12N 9/90; C12N 15/11; C12N 2310/20; C12N 2310/315; C12N 2310/321; C12N 2310/322; C12N 2310/531

(57) ABSTRACT

Provided are methods, compositions, and systems for targeted pseudouridylation of RNA. In some aspects, provided are methods for editing a target RNA (e.g., mRNA) in a host cell, comprising introducing an engineered guide small nucleolar RNA (gsnoRNA) into the host cell, wherein the gsnoRNA recruits a DKC1 protein to modify a target uridine residue into a pseudouridine residue in the target RNA. In some embodiments, the DKC1 protein has cytoplasmic localization in the host cell.

### CROSS-REFERENCE TO RELATED APPLICATIONS

This application claims priority benefit of International Patent Application No. PCT/CN2021/096122 filed May 26, 2021, the content of which is incorporated herein by reference in its entirety.

### INCORPORATION BY REFERENCE OF SEQUENCE LISTING

The content of the following submission on ASCII text file is incorporated herein by reference in its entirety: a computer readable form (CRF) of the Sequence Listing (file name: 165392000441SEQLIST.TXT, date recorded: May 24, 2022, size: 75,959 bytes).

### FIELD

The present application relates to compositions, systems and methods for editing RNA via targeted pseudouridylation using DKC1.

### BACKGROUND

Pseudouridine (Ψ) is the most abundant post-transcriptionally modified nucleotide in stable RNAs, including tRNA, rRNA, snRNA and mRNA, constituting approximately 5% of total ribonucleotides. The conversion of uridine to Y (pseudouridylation) requires two distinct chemical reactions: the breaking of the C1′-N1 glycosydic bond and the making of a new carbon-glycosydic (C1′-C5) bond that relinks the base to the sugar. Pseudouridylation is a true isomerization reaction, which creates an extra hydrogen bond donor and influences a wide variety of functional aspects depending on the type of RNA that carries the Y and the position within the RNA sequence, such as protein synthesis and increased stop-codon read-through (Yu and Meier, 2014, RNA Biology 11:1483-1494). Many of the mRNA Ψs reside in coding regions, and the majority of them respond to environmental stress, indicating functional significance (Carlile et al. 2014, Nature 515:143).

In eukaryotes and archaea, pseudouridylation can be introduced by box H/ACA ribonucleoproteins (RNPs), each of which contains a unique small RNA (box H/ACA RNA, one of the two major classes of small nucleolar RNAs, or ‘snoRNAs’) and four core proteins (dyskerin (DKC1), NHP2, NOP10 and GAR1). Dyskerin (DKC1; also known as NAP57/CBF5) is a highly conserved multifunctional protein that acts as RNA-guided pseudouridine synthase, directing the enzymatic conversion of specific uridines to pseudouridines. It concentrates in the nucleoli and the Cajal bodies (CBs) where, in association with three other highly conserved proteins—Nop10, Nhp2, Gar1—composes a tetramer able to enter in the composition of different nuclear RNPs playing key biological functions. Within the nucleolus, the tetramer associates with H/ACA small nucleolar RNAs (snoRNAs) to compose the H/ACA snoRNPs, that regulate rRNA processing and pseudouridylate RNA targets by snoRNA-guided base complementarity. Within the CBs, it associates with CB specific small RNAs (scaRNAs) to compose the scaRNPs, that direct pseudouridylation of spliceosomal snRNAs. NAP57/dyskerin (DKC1)/CBF5 catalyzes the chemical reactions, converting the target uridine to Ψ. The RNA component serves as a guide that specifies, through base-pairing interaction with its substrate RNA, the target uridine for pseudouridylation (Ge and Yu, 2013, Trends Biochem Sci 38(4):210-218). Based on this guide-substrate base-pairing scheme, Karijolich and Yu (2011, Nature 474:395-398) designed an artificial box H/ACA RNA to introduce Ψ into mRNA at a Premature Termination Codon (PTC) in S. cerevisiae. They demonstrated that Ψ was indeed incorporated into TRM4 mRNA at the PTC. Pseudouridylated PTC promoted nonsense suppression by altering ribosome decoding (Fernandez et al. 2013, Nature 500:107-110; Wu et al. 2015, Methods in Enzymology 560:187-217; U.S. Pat. No. 8,603,457). Using a similar strategy, others showed that artificial H/ACA RNAs could site-specifically pseudouridylate pre-mRNA after microinjection into Xenopus oocytes (Chen et al. 2010, Mol Cell Biol 30:4108-4119). In both examples, the artificial H/ACA RNAs were modified to alter the loops that serve as the guide sequence, but otherwise these snoRNAs were unaltered.

Although site-specific pseudouridylation or target RNAs is a potentially powerful technique, the methods available thus far have resulted in low editing efficiency of target RNAs. Accordingly, there is a need in the art for optimized gsnoRNAs, gsnoRNA-based gene editing systems, and methods of editing a target RNA by pseudouridylation.

### BRIEF SUMMARY

The present application provides methods for editing target RNAs in host cells using gsnoRNA and DKC1 protein. Embodiments of the methods are also referred herein as the “RESTART” method, which can be used to allow read-through of RNA transcripts having a premature termination codons (PTC).

In some aspects, the present application provides a method for editing a target RNA in a host cell, comprising introducing a guide small nucleolar RNA (gsnoRNA) and a nucleic acid molecule encoding a DKC1 protein into the host cell, wherein the gsnoRNA comprises a guide sequence that hybridizes to a sequence comprising a target uridine residue in the target RNA, and wherein the gsnoRNA recruits the DKC1 protein to modify the target uridine residue into a pseudouridine residue in the target RNA. In some embodiments, the gsnoRNA comprises a scaffold sequence derived from a wildtype H/ACA-snoRNA selected from the group consisting of ACA19, ACA2b, ACA36, ACA44, ACA27, E2, ACA3, and ACA17.

In some aspects, provided herein is a method for editing a target RNA in a host cell, comprising introducing an engineered gsnoRNA into the host cell, wherein the gsnoRNA comprises a guide sequence that hybridizes to a sequence comprising a target uridine residue in the target RNA, wherein the gsnoRNA comprises a scaffold sequence derived from wildtype ACA2b, ACA36, ACA44, ACA27, E2, ACA3, or ACA17, and wherein the gsnoRNA recruits a DKC1 protein in the host cell to modify the target uridine residue into a pseudouridine residue in the target RNA. In some embodiments, the DKC1 protein is an endogenous DKC1 protein of the host cell. In some embodiments, the method further comprises introducing a nucleic acid encoding the DKC1 protein into the host cell.

In some aspects, provided herein is a method for editing a target RNA in a host cell, comprising introducing an engineered gsnoRNA into the host cell, wherein the gsnoRNA comprises a guide sequence that hybridizes to a sequence comprising a target uridine residue in the target RNA, wherein the gsnoRNA comprises a nucleotide sequence selected from the group consisting of the nucleotide sequences provided in Table 2, Table 3, or Table 4, and wherein the gsnoRNA recruits a DKC1 protein in the host cell to modify the target uridine residue into a pseudouridine residue in the target RNA.

In some aspects, provided herein is a method for editing a target RNA in a host cell, comprising introducing an engineered gsnoRNA into the host cell, wherein the gsnoRNA comprises a guide sequence that hybridizes to a sequence comprising a target uridine residue in the target RNA, wherein the gsnoRNA comprises a nucleotide sequence selected from the group consisting of SEQ ID NOs: 4-6, 9-12, 15-19, 22-36, and 177-179 and wherein the gsnoRNA recruits a DKC1 protein in the host cell to modify the target uridine residue into a pseudouridine residue in the target RNA.

In some aspects, provided herein is a method for editing a target RNA in a host cell, comprising introducing an engineered gsnoRNA into the host cell, wherein the gsnoRNA comprises a guide sequence that hybridizes to a sequence comprising a target uridine residue in the target RNA, wherein the gsnoRNA comprises a nucleotide sequence selected from the group consisting of SEQ ID NOs: 20-21 and 145-150, and wherein the gsnoRNA recruits a DKC1 protein in the host cell to modify the target uridine residue into a pseudouridine residue in the target RNA. In some embodiments, the DKC1 protein is an endogenous DKC1 protein of the host cell. In some embodiments, the method further comprises introducing a nucleic acid encoding the DKC1 protein into the host cell.

In some embodiments according to any of the methods described above, the DKC1 protein has cytoplasmic localization in the host cell.

In some embodiments according to any of the methods described above, the DKC1 protein comprises a DKC1 protein fragment corresponding to amino acid residues 41 to 420 of a human DKC1 isoform 3 protein, wherein the amino acid numbering is according to SEQ ID NO: 2.

In some embodiments according to any of the methods described above, the DKC1 protein comprises an amino acid sequence having at least 85% (e.g., at least about any one of 90%, 91%, 92%, 93%, 94%, 95%, 96%, 97%, 98%, 99%, or more) identity to SEQ ID NO: 88. In some embodiments, the DKC1 protein comprises the amino acid sequence of SEQ ID NO: 88. In some embodiments according to any of the methods described above, the DKC1 protein comprises a naturally occurring DKC1 isoform with cytoplasmic localization in the host cell.

In some aspects, provided herein is a method for editing a target RNA in a host cell, comprising introducing an engineered gsnoRNA into the host cell, wherein the gsnoRNA comprises a guide sequence that hybridizes to a sequence comprising a target uridine residue in the target RNA, wherein the host cell expresses a DKC1 isoform with cytoplasmic localization, and wherein the gsnoRNA recruits the DKC1 isoform to modify the target uridine residue into a pseudouridine residue in the target RNA.

In some aspects, provided herein is a method for editing a target RNA in a host cell, comprising introducing (a) an engineered gsnoRNA and (b) a splice-switching antisense oligonucleotide (ASO) into the host cell, wherein the gsnoRNA comprises a guide sequence that hybridizes to a sequence comprising a target uridine residue in the target RNA, wherein the ASO enhances expression of a DKC1 protein that is an endogenous DKC1 isoform with cytoplasmic localization in the host cell, and wherein the gsnoRNA recruits the DKC1 protein to modify the target uridine residue into a pseudouridine residue in the target RNA. In some embodiments, the gsnoRNA comprises a scaffold sequence derived from a wildtype H/ACA-snoRNA selected from the group consisting of ACA19, ACA2b, ACA36, ACA44, ACA27, E2, ACA3, and ACA17.

In some embodiments according to any of the methods described above, the DKC1 isoform corresponds to isoform 3 of human DKC1 protein.

In some embodiments according to any of the methods described above, the DKC1 protein comprises an amino acid sequence having at least 85% (e.g., at least about any one of 90%, 91%, 92%, 93%, 94%, 95%, 96%, 97%, 98%, 99%, or more) identity to SEQ ID NO: 2. In some embodiments, the DKC1 protein comprises the amino acid sequence of SEQ ID NO: 2.

In some embodiments according to any of the methods described above, the DKC1 protein comprises a DKC1 protein fragment corresponding to amino acid residues 1 to 419 of a full-length human DKC1 isoform 1 protein, wherein the amino acid numbering is according to SEQ ID NO: 1.

In some embodiments according to any of the methods described above, the gsnoRNA comprises a scaffold sequence is derived from ACA2b. In some embodiments according to any of the methods described above, the gsnoRNA comprises a scaffold sequence derived from ACA36. In some embodiments, the gsnoRNA comprises a mutation in the 3′ hairpin of the ACA36 scaffold.

In some embodiments according to any of the methods described above, the gsnoRNA comprises a scaffold sequence derived from ACA19.

In some embodiments according to any of the methods described above, the gsnoRNA comprises one or more guide sequences each located in a region corresponding to a hairpin structure of the wildtype H/ACA-snoRNA. In some embodiments, at least one of the one or more guide sequences is located in a hairpin structure at the 3′ terminal part (also referred herein as “3′ hairpin structure”) of the wildtype H/ACA-snoRNA. In some embodiments, at least one of the one or more guide sequences is located in a hairpin structure at the 5′ terminal part (also referred herein as “5′ hairpin structure”) of the wildtype H/ACA-snoRNA. In some embodiments, the gsnoRNA comprises a single guide sequence. In some embodiments, the gsnoRNA comprises two or more (e.g., 2, 3, 4, 5, 6, or more) guide sequences.

In some embodiments according to any of the methods described above, the gsnoRNA comprises one or more mutations (e.g., substitution, insertion and/or deletion) in one or more hairpin structures (e.g., the 3′ and/or 5′ hairpin structures) of the wildtype ACA19.

In some embodiments according to any of the methods described above, the engineered gsnoRNA comprises one or more substitution mutations in nucleotides of a polyU sequence in the wildtype H/ACA-snoRNA, wherein the polyU sequence comprises at least 4 consecutive U residues.

In some embodiments according to any of the methods described above, the engineered gsnoRNA comprises one or more insertion or deletion mutations positioned between the nucleotide residue in the guide region that hybridizes to the target uridine and an H/ACA box of the wildtype H/ACA snoRNA, whereby the engineered gsnoRNA comprises 14 or 15 nucleotides between the nucleotide residue in the guide region that hybridizes to the target uridine and the H/ACA box.

In some embodiments, the one or more mutations are selected from the group consisting of substitution of residues 26-29 with UUCU, substitution of residues 26-29 with UGUU, addition of G to the 3′ hairpin structure after residue 115, and addition of a dinucleotide sequence (XX, e.g., CU) to the 5′ hairpin after residue 8, wherein X is a nucleotide selected from A, U, C, and G, and wherein the numbering is according to SEQ ID NO: 37. In some embodiments, the dinucleotide sequence is part of the guide RNA designed to hybridize to the target RNA.

In some embodiments according to any of the methods described above, the gsnoRNA comprises a nucleotide sequence selected from the group consisting of SEQ ID NOs: 3-12, 15-19, 22-36, and 177-179. In some embodiments, the gsnoRNA comprises a nucleotide sequence selected from the group consisting of SEQ ID NOs: 15-19.

In some embodiments according to any of the methods described above, the gsnoRNA comprises a nucleotide sequence selected from the group consisting of SEQ ID NOs: 20-21 and 145-150.

In some embodiments according to any of the methods described above, the method comprises introducing a nucleic acid molecule encoding the gsnoRNA into the host cell. In some embodiments, the nucleic acid molecule encoding the gsnoRNA is under a small RNA promoter. In some embodiments, the nucleic acid molecule encoding the gsnoRNA is under a promoter selected from the group consisting of U6 (e.g., transcribed by Polymerase III) and U1 (e.g., transcribed by Polymerase II) promoters. In some embodiments, the nucleic acid molecule encoding the gsnoRNA is embedded in an intron sequence located between a first exon sequence and a second exon sequence, and wherein the first exon sequence, the intron sequence and the second exon sequence are derived from a naturally-occurring gene. In some embodiments, the intron sequence is from an intron of an endogenous gene in the host cell, wherein the gene is selected from the group consisting of EIF3A, SNHG12, RPL21, and RPSA. In some embodiments, the intron sequence is from an intron of an exogenous gene, such as HBB. In some embodiments, the nucleic acid encoding the gsnoRNA is not embedded in an intron sequence.

In some embodiments according to any of the methods described above, the nucleic acid molecule encoding the DKC1 protein is present in a viral vector. In some embodiments, the nucleic acid molecule encoding the gsnoRNA is present in a viral vector.

In some embodiments according to any of the methods described above, the method comprises introducing into the host cell a vector comprising a first nucleic acid sequence encoding the DKC1 protein and a second nucleic acid sequence encoding the gsnoRNA. In some embodiments, the nucleic acid molecule encoding the DKC1 protein and the nucleic acid molecule encoding the gsnoRNA are present in separate vectors. In some embodiments, the vector is a viral vector. In some embodiments, the vector is an adeno-associated viral (AAV) vector.

In some embodiments according to any of the methods described above, the gsnoRNA comprises one or more chemically modified nucleosides and/or inter-nucleosidic linkages. In some embodiments, the gsnoRNA comprises one or more nucleosides having 2′-OMe or 2′-MOE modifications. In some embodiments, the gsnoRNA comprises no more than 10, no more than 8, no more than 6, or no more than 4 chemically modified nucleosides. In some embodiments, the gsnoRNA comprises one or more phosphorothioate inter-nucleosidic linkages. In some embodiments, the gsnoRNA comprises no more than 10, no more than 9, no more than 8, or no more than 6 phosphorothioate inter-nucleosidic linkages. In some embodiments, the gsnoRNA comprises a 5′ cap modification. In some embodiments, the 5′ cap modification is a 7-methylguanosine (m⁷G) cap. In some embodiments, the gsnoRNA does not comprise one or more chemically modified nucleosides or inter-nucleosidic linkages. [symbol] cap.

[symbol]

In some embodiments according to any of the methods described above, efficiency of editing the target RNA is at least 10% (e.g., at least about any one of 15%, 20%, 25%, 30%, 35%, 40%, 45%, 50%, 60%, or higher).

In some embodiments according to any of the methods described above, wherein the sequence comprising the target uridine in the target RNA is a premature termination codon in a sequence encoding a protein, the method results in expression of the full-length protein in the host cell of at least 4% (e.g., at least 5%, at least 6%, at least 7%, at least 8%, at least 9% or at least 10%) of the expression level of the full-length protein without a premature termination codon.

In some embodiments according to any of the methods described above, wherein the sequence comprising the target uridine in the target RNA is a premature termination codon in a sequence encoding a protein, the method results in expression of the full-length protein, wherein the expression of the protein is detectable without enrichment (e.g., without enrichment by immunoprecipitation). In some embodiments, the protein is detected via a tag (e.g., via a fluorescent tag). In some embodiments, the protein is detected by immo-staining according to methods known in the art.

In some embodiments according to any of the methods described above, wherein the sequence comprising the target uridine in the target RNA is a premature termination codon in a sequence encoding a protein, the method results in expression of the full-length protein in at least 20% of host cells (e.g., at least 25%, at least 30%, at least 35%, at least 40%, at least 45%, or at least 50% of host cells).

In some embodiments according to any of the methods described above, the target RNA is not a ribosomal RNA (rRNA) such as an endogenous rRNA of the host cell.

In some embodiments according to any of the methods described above, the target RNA is a messenger RNA (mRNA). In some embodiments, the sequence comprising the target uridine in the target RNA is a stop codon, and modification of the target uridine to pseudouridine causes the stop codon to be translated as a coding codon. In some embodiments, the stop codon is a premature termination codon (PTC). In some embodiments, the PTC is associated with a genetic disease or condition. In some embodiments, the sequence comprising the target uridine in the target RNA is a stop codon, and modification of the target uridine to pseudouridine reduces or prevents nonsense-mediate decay (NMD).

In some embodiments according to any of the methods described above, the DKC1 protein is part of a ribonucleoprotein (RNP) complex that associates with the gsnoRNA. In some embodiments, the RNP complex comprises NOP10, GAR1, and NHP2.

In some embodiments according to any of the methods described above, the host cell is an archaeal cell. In some embodiments, the host cell is a eukaryotic cell. In some embodiments, the host cell is a mammalian cell. In some embodiments, the host cell is a human cell.

In some embodiments according to any of the methods described above, the method is carried out in vivo. In some embodiments, the method is carried out ex vivo.

In some aspects, provided herein is a method of treating a disease or condition associated with a PTC in a target RNA in a subject, comprising editing the target RNA in a cell of the subject using any of the methods described above, wherein the gsnoRNA comprises a guide sequence that hybridizes to the PTC in the target RNA, and wherein modification of the uridine residue in the PTC to a pseudouridine residue causes translation read-through of the PTC in the target RNA, thereby treating the disease or condition in the subject.

In some embodiments, the disease or condition is selected from the group consisting of Cystic fibrosis, Hurler Syndrome, alpha-1-antitrypsin (A1AT) deficiency, Parkinson's disease, Alzheimer's disease, albinism, Amyotrophic lateral sclerosis, Asthma, 8-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase, Haemophilia, Hereditary Hemochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer.

In some aspects, provided herein is an engineered gsnoRNA comprising a guide sequence that hybridizes to a sequence comprising a target uridine residue in a target RNA in a host cell. In some embodiments, the gsnoRNA comprises a nucleotide sequence selected from the group consisting of SEQ ID NOs: 4-6, 9-12, 15-19, 22-36, and 177-179, and the gsnoRNA is capable of recruiting a DKC1 protein in the host cell to modify the target uridine residue into a pseudouridine residue in the target RNA. In some embodiments, the gsnoRNA comprises a nucleotide sequence selected from the group consisting of SEQ ID NOs: 20-21 and 145-150, and the gsnoRNA is capable of recruiting a DKC1 protein in the host cell to modify the target uridine residue into a pseudouridine residue in the target RNA.

In some aspects, provided herein is an engineered gsnoRNA comprising a guide sequence that hybridizes to a sequence comprising a target uridine residue in a target RNA in a host cell, wherein the gsnoRNA comprises a scaffold sequence derived from a wildtype H/ACA-snoRNA selected from the group consisting of ACA2b, ACA36, ACA44, ACA27, E2, ACA3, and ACA17, and wherein the gsnoRNA is capable of recruiting a DKC1 protein in the host cell to modify the target uridine residue into a pseudouridine residue in the target RNA.

In some embodiments according to any of the engineered gsnoRNAs described above, the gsnoRNA comprises a 5′ cap modification. In some embodiments, the 5′ cap modification is a 7-methylguanosine (m⁷G) cap. In some embodiments according to any of the engineered gsnoRNAs described above, the gsnoRNA comprises one or more chemically modified nucleosides and/or inter-nucleosidic linkages. In some embodiments according to any one of the engineered gsnoRNAs described above, the gsnoRNA comprises one or more nucleosides having 2′-OMe or 2′-MOE modifications. In some embodiments, the gsnoRNA comprises no more than 10, no more than 8, no more than 6, or no more than 4 chemically modified nucleosides. In some embodiments, the gsnoRNA comprises one or more phosphorothioate inter-nucleosidic linkages. In some embodiments, the gsnoRNA comprises no more than 10, no more than 9, no more than 8, or no more than 6 phosphorothioate inter-nucleosidic linkages.

In some embodiments according to any of the engineered gsnoRNAs described above, the gsnoRNA comprises a scaffold sequence is derived from ACA2b. In some embodiments according to any of the engineered gsnoRNAs described above, the gsnoRNA comprises a scaffold sequence derived from ACA36. In some embodiments, the gsnoRNA comprises a mutation in the 3′ hairpin of the ACA36 scaffold.

In some embodiments according to any of the engineered gsnoRNAs described above, the gsnoRNA comprises a scaffold sequence derived from ACA19.

In some embodiments according to any of the engineered gsnoRNAs described above, the gsnoRNA comprises one or more guide sequences each located in a region corresponding to a hairpin structure of the wildtype H/ACA-snoRNA. In some embodiments, at least one of the one or more guide sequences is located in a hairpin structure at the 3′ terminal part of the wildtype H/ACA-snoRNA. In some embodiments, at least one of the one or more guide sequences is located in a hairpin structure at the 5′ terminal part of the wildtype H/ACA-snoRNA. In some embodiments, the gsnoRNA comprises a single guide sequence. In some embodiments, the gsnoRNA comprises two or more (e.g., 2, 3, 4, 5, 6, or more) guide sequences.

In some embodiments according to any of the engineered gsnoRNAs described above, the gsnoRNA comprises one or more mutations (e.g., substitution, insertion and/or deletion) in one or more hairpin structures (e.g., the 3′ and/or 5′ hairpin structures) of the wildtype ACA19.

In some embodiments according to any of the engineered gsnoRNAs described above, the engineered gsnoRNA comprises one or more substitution mutations in nucleotides of a polyU sequence in the wildtype H/ACA-snoRNA, wherein the polyU sequence comprises at least 4 consecutive U residues.

In some embodiments according to any of the engineered gsnoRNAs described above, the engineered gsnoRNA comprises one or more insertion or deletion mutations positioned between the nucleotide residue in the guide region that hybridizes to the target uridine and an H/ACA box of the wildtype H/ACA snoRNA, whereby the engineered gsnoRNA comprises 14 or 15 nucleotides between the nucleotide residue in the guide region that hybridizes to the target uridine and the H/ACA box.

In some embodiments, the one or more mutations are selected from the group consisting of substitution of residues 26-29 with UUCU, substitution of residues 26-29 with UGUU, addition of G to the 3′ hairpin structure after residue 115, and addition of a dinucleotide sequence (XX, e.g., CU) to the 5′ hairpin after residue 8, wherein X is a nucleotide selected from A, U, C, and G, and wherein the numbering is according to SEQ ID NO: 37. In some embodiments, the engineered gsnoRNA comprises a nucleotide sequence selected from the group consisting of SEQ ID NOs: 15-19.

In some aspects, provided herein is an engineered gsnoRNA comprising a guide sequence that hybridizes to a sequence comprising a target uridine residue in a target RNA in a host cell, wherein the gsnoRNA comprises a scaffold sequence derived from a wildtype ACA19, wherein the engineered gsnoRNA comprises one or more insertion or deletion mutations positioned between the nucleotide residue in the guide region that hybridizes to the target uridine and an H/ACA box of the wildtype ACA19, wherein the engineered gsnoRNA comprises 14 or 15 nucleotides between the nucleotide residue in the guide region that hybridizes to the target uridine and the H/ACA box, and wherein the gsnoRNA is capable of recruiting a DKC1 protein in the host cell to modify the target uridine residue into a pseudouridine residue in the target RNA. In some embodiments, the one or more mutations are selected from the group consisting of the one or more mutations are selected from the group consisting of substitution of residues 26-29 with UUCU, substitution of residues 26-29 with UGUU, addition of G to the 3′ hairpin structure after residue 115, and addition of a dinucleotide sequence (XX, e.g., CU) to the 5′ hairpin after residue 8, wherein X is a nucleotide selected from A, U, C, and G, and wherein the numbering is according to SEQ ID NO: 37. In some embodiments, the gsnoRNA comprises a nucleotide sequence selected from the group consisting of SEQ ID NOs: 15-19.

In some aspects, provided herein is an isolated nucleic acid molecule comprising a sequence encoding the engineered gsnoRNA of any of the preceding embodiments. In some embodiments, provided herein is a vector (e.g., viral vector) comprising the nucleic acid molecule.

In some aspects, provided herein is an engineered RNA-editing system comprising: (a) a gsnoRNA comprising a guide sequence that hybridizes to a sequence comprising a target uridine residue in a target RNA in a host cell, or a nucleic acid molecule encoding the gsnoRNA; and (b) a DKC1 protein, or a nucleic acid molecule encoding the DKC1 protein, wherein the gsnoRNA is capable of recruiting the DKC1 protein to modify the target uridine residue into a pseudouridine residue in the target RNA.

In some embodiments, the DKC1 protein has cytoplasmic localization in the host cell. In some embodiments according to any of the methods described above, the DKC1 protein comprises a DKC1 protein fragment corresponding to amino acid residues 41 to 420 of a human DKC1 isoform 3 protein, wherein the amino acid numbering is according to SEQ ID NO: 2.

In some embodiments according to any of the methods described above, the DKC1 protein comprises an amino acid sequence having at least 85% (e.g., at least about any one of 90%, 91%, 92%, 93%, 94%, 95%, 96%, 97%, 98%, 99%, or more) identity to SEQ ID NO: 88. In some embodiments, the DKC1 protein comprises the amino acid sequence of SEQ ID NO: 88. In some embodiments according to any of the methods described above, the DKC1 protein comprises a naturally occurring DKC1 isoform with cytoplasmic localization in the host cell.

In some embodiments according to any of the engineered RNA-editing systems described above, the DKC1 isoform corresponds to isoform 3 of human DKC1 protein.

In some embodiments according to any of the engineered RNA-editing systems described above, the DKC1 protein comprises an amino acid sequence having at least 85% (e.g., at least about any one of 90%, 91%, 92%, 93%, 94%, 95%, 96%, 97%, 98%, 99%, or more) identity to SEQ ID NO: 2. In some embodiments, , the DKC1 protein comprises the amino acid sequence of SEQ ID NO: 2.

In some embodiments according to any of the engineered RNA-editing systems described above, the DKC1 protein comprises a DKC1 protein fragment corresponding to amino acid residues 1 to 419 of a full-length human DKC1 isoform 1 protein, wherein the amino acid numbering is according to SEQ ID NO: 1.

In some embodiments according to any of the engineered RNA-editing systems described above, the gsnoRNA comprises a scaffold sequence is derived from ACA2b. In some embodiments according to any of the engineered RNA-editing systems described above, the gsnoRNA comprises a scaffold sequence derived from ACA36. In some embodiments, the gsnoRNA comprises a mutation in the 3′ hairpin of the ACA36 scaffold.

In some embodiments according to any of the engineered RNA-editing systems described above, the gsnoRNA comprises a scaffold sequence derived from ACA19.

In some embodiments according to any of the engineered RNA-editing systems described above, the gsnoRNA comprises one or more guide sequences each located in a region corresponding to a hairpin structure of the wildtype H/ACA-snoRNA. In some embodiments, at least one of the one or more guide sequences is located in a hairpin structure at the 3′ terminal part of the wildtype H/ACA-snoRNA. In some embodiments, at least one of the one or more guide sequences is located in a hairpin structure at the 5′ terminal part of the wildtype H/ACA-snoRNA. In some embodiments, the gsnoRNA comprises a single guide sequence. In some embodiments, the gsnoRNA comprises two or more (e.g., 2, 3, 4, 5, 6, or more) guide sequences.

In some embodiments according to any of the engineered RNA-editing systems described above, the gsnoRNA comprises one or more mutations (e.g., substitution, insertion and/or deletion) in one or more hairpin structures (e.g., the 3′ and/or 5′ hairpin structures) of the wildtype ACA19.

In some embodiments according to any of the engineered RNA-editing systems described above, the engineered gsnoRNA comprises one or more substitution mutations in nucleotides of a polyU sequence in the wildtype H/ACA-snoRNA, wherein the polyU sequence comprises at least 4 consecutive U residues.

In some embodiments according to any of the engineered RNA-editing systems described above, the engineered gsnoRNA comprises one or more insertion or deletion mutations positioned between the nucleotide residue in the guide region that hybridizes to the target uridine and an H/ACA box of the wildtype H/ACA snoRNA, whereby the engineered gsnoRNA comprises 14 or 15 nucleotides between the nucleotide residue in the guide region that hybridizes to the target uridine and the H/ACA box.

In some embodiments of the engineered RNA-editing system, the DKC1 protein is part of a ribonucleoprotein (RNP) complex that associates with the gsnoRNA. In some embodiments, the ribonucleoprotein complex comprises NOP10, GAR1, and/or NHP2.

In some embodiments, the one or more mutations are selected from the group consisting of the one or more mutations are selected from the group consisting of substitution of residues 26-29 with UUCU, substitution of residues 26-29 with UGUU, addition of G to the 3′ hairpin structure after residue 115, and addition of a dinucleotide sequence (XX, e.g., CU) to the 5′ hairpin after residue 8, wherein X is a nucleotide selected from A, U, C, and G, and wherein the numbering is according to SEQ ID NO: 37.

In some embodiments according to any of the engineered RNA-editing systems described above, the gsnoRNA comprises a nucleotide sequence selected from the group consisting of SEQ ID NOs: 3-12, 15-19, 22-36, and 177-179. In some embodiments, the gsnoRNA comprises a nucleotide sequence selected from the group consisting of SEQ ID NOs: 15-19.

In some embodiments according to any of the engineered RNA-editing systems described above, the gsnoRNA comprises a nucleotide sequence selected from the group consisting of SEQ ID NOs: 20-21 and 145-150.

In some aspects, provided herein is a pharmaceutical composition comprising any of the gsnoRNA, the nucleic acid molecules, or the engineered RNA-editing systems described above, and a pharmaceutically acceptable carrier.

In some aspects, provided herein is a host cell comprising any of the gsnoRNA, the nucleic acid molecules, or the engineered RNA-editing systems described above.

In some aspects, provided herein is a kit for editing a target RNA in a host cell, comprising any of the gsnoRNA, the nucleic acid molecules, or the engineered RNA-editing systems described above.

Also provided are compositions, kits and articles of manufacture for use in any one the methods described above.

### BRIEF DESCRIPTION OF THE DRAWINGS

FIGS. 1A-1F show readthrough of premature termination codon mediated by engineered guide snoRNA. FIG. 1A provides schematics of the “RESTART” method design. The snoRNP complex is indicated in dashed box. FIG. 1B provides schematics showing the structure of Reporter-1 and guide snoRNA constructs. In the Reporter-1, 15 bases are inserted into the position between the codons of 154^{th} and 155^{th} amino acids. The DNA sequences of 15 bases are shown, and the premature termination codon (PTC) site (TAG) is indicated. A positive control (Venus-GGT) is included. FIGS. 1C-1D, HEK293T cells were co-transfected with Reporter-1 and guide snoRNA constructs. Venus expression was detected by high-content imaging system. FIG. 1C shows representative fluorescence images of cells showing the expression levels of Venus. Bar, 200 μm. FIG. 1D shows a dot plot showing the relative fraction of Venus positive cells. FIG. 1E, Western blot analysis showing expression levels of DKC1 proteins upon DKC1 stable knockdown. FIG. 1F, Bar plot showing the relative fraction of Venus positive cells in shControl and DKC1 stable knockdown cells co-transfected with Reporter-1 and gsnoRNA constructs.

FIGS. 2A-2C show the PTC-readthrough effects mediated by gsnoRNAs of different constructs. Dot plots showing the relative fraction of Venus positive cells co-transfected with Reporter-1 and gsnoRNAs within the host intron (FIG. 2A), gsnoRNAs within HBB intron (FIG. 2B), or gsnoRNAs transcribed from small RNA promoter (FIG. 2C). The structure of gsnoRNA constructs were indicated in bottom of each panel.

FIGS. 3A-3F show predicted secondary structure of gsnoRNA scaffolds used in FIGS. 1A-1F. The secondary structures and base pair probabilities are predicted using the RNAfold server, as described in Gruber et al. (The Vienna RNA websuite. Nucleic Acids Res 36, W70-4 (2008)), the contents of which are herein incorporated by reference in their entirety.

FIGS. 4A-4F show optimization of gsnoRNA scaffolds improves the efficiency of PTC-readthrough. FIG. 4A, Predicted secondary structure of gACA19, gACA2b, and gACA36 scaffolds. The secondary structures and base pair probabilities are predicted using the RNAfold server. In the structure of gACA19 scaffold, seven mutations are indicated. FIG. 4B, Structure of the gsnoRNA construct. FIG. 4C, Dot plot showing the relative fraction of Venus positive cells co-transfected with Reporter-1 and gsnoRNAs of (FIG. 4B) constructs. FIG. 4D, Representative fluorescence images of cells co-transfected with Reporter-1 and gsnoRNAs of (FIG. 4B) constructs. Bar, 200 μm. FIG. 4E, Dot plot showing the relative fraction of Venus positive cells co-transfected with Reporter-1 and engineered gACA19 scaffolds with different mutations. The engineered positions of gACA19 are annotated in (FIG. 4A). FIG. 4F, Representative fluorescence images of (FIG. 4E). Bar, 200 μm.

FIGS. 5A-5D show predicted secondary structure of gsnoRNA scaffolds used in FIGS. 4A-4F. The secondary structures and base pair probabilities are predicted using the RNAfold server.

FIGS. 6A-6B show engineering of gACA36 scaffolds. FIG. 6A, Predicted secondary structure of engineered gACA36 scaffolds. The secondary structures and base pair probabilities are predicted using the RNAfold server. FIG. 6B, Dot plot showing the relative fraction of Venus positive cells co-transfected with Reporter-1 and different gsnoRNAs constructs.

FIGS. 7A-7I show that exogenous DKC1-isoform3 protein improves the efficiency of PTC-RT. FIG. 7A, Structure of two isoforms of human DKC1 transcripts. Exons are numbered on top, coding regions are represented by filled boxes, and UTRs are represented by white boxes. NLS, nuclear localization signal. FIG. 7B, Schematic showing the structure of Reporter-3 construct, in which gsnoRNA is arranged in tandem with reporter. The sequences surrounding the PTC sites and the PTC sites (TAA/TAG/TGA) are shown. FIG. 7C, Western blot analysis showing expression levels of DKC1 proteins in HEK293T DKC1 stable overexpression cells. Santa Cruz and Abcam anti-DKC1 antibodies target the C-terminal and N-terminal region of DKC1 protein, respectively. FIGS. 7D-7F, Indicated Reporter-3 constructs were transfected into control HEK293T, DKC1-isoform1 stable overexpression, and DKC1-isoform3 stable overexpression cells, respectively. FIG. 7D, Representative fluorescence images of cells. Bar, 200 μm. FIG. 7E, Bar plot showing the relative fraction of EGFP positive cells. FIG. 7F, Bar plot showing the relative fraction of EGFP intensities. FIG. 7G, Bar plot showing the relative fraction of EGFP positive cells in HEK293T cells transfected with different Reporter-3 constructs, and co-transfected with different Reporter-3 and DKC1-isoform3 (200 ng) constructs. FIG. 7H, Bar plot showing the relative fraction of EGFP intensities in HEK293T cells transfected with different Reporter-3 constructs, and co-transfected with different Reporter-3 and DKC1-isoform3 (200 ng) constructs. FIG. 7I, Locus-specific Ψ modifications in Reporter-3 transcripts were detected by a radiolabeling-free, qPCR-based method. The curves were obtained by high-resolution melting analysis. Ψ site is specifically labeled by CMC chemical; after reverse transcription, the Ψ-CMC adduct cause a mutation/deletion at or around Ψ site in cDNA, thus giving rise to a shift in the melting temperature. HEK293T cells were co-transfected with different Reporter-3 and DKC1-isoform3 (200 ng) constructs.

FIGS. 8A-8D show that exogenous DKC1-isoform3 protein improves the efficiency of PTC-RT. FIGS. 8A-8C, Reporter-1 and gsnoRNA constructs were co-transfected into control HEK293T, DKC1-isoform1 stable overexpression, and DKC1-isoform3 stable overexpression cells, respectively. FIG. 8A, Representative fluorescence images of cells. Bar, 200 μm. FIG. 8B, Bar plot showing the relative fraction of Venus positive cells. FIG. 8C, Bar plot showing the relative fraction of Venus intensities. FIG. 8D, Dot plot showing the relative fraction of EGFP positive cells co-transfected with Reporter-3 together with empty vector (Vec), DKC1-isoform1 (DKC1 iso1) or DKC1-isoform3 (DKC1 iso3) constructs. The statistical analyses of bar plots are unpaired Student's t-tests.

FIG. 9 shows readthrough efficiency of different truncation of DKC1-isoform3. Dot plot showing the relative fraction of EGFP positive cells co-transfected with Reporter-3 and different DKC1-isoform3 truncation constructs.

FIGS. 10A-10E show comparison of readthrough efficiencies on different stop codons. FIG. 10A, Representative fluorescence images of cells transfected with different Reporter-3 constructs. Bar, 200 μm. FIG. 10B, Representative fluorescence images of cells co-transfected with indicated Reporter-3 and DKC1-isoform3 (200 ng) constructs. Bar, 200 μm. FIGS. 10C-10E, Bar plots showing the relative fraction of the EGFP positive cells co-transfected with Reporter-3-TAA (FIG. 10C), Reporter-3-TAG (FIG. 10D), or Reporter-3-TGA (FIG. 10E) together with decreasing amount of DKC1-isoform3 constructs.

FIGS. 11A-11C show detection of locus-specific Ψ modifications. HEK293T cells were co-transfected with different Reporter-3 and DKC1-isoform3 (200 ng) constructs. Locus-specific Ψ modifications in Reporter-3 transcripts (FIG. 11A) and Ψ1045 sites in 18S rRNA (FIGS. 11B-11C) were detected by a radiolabeling-free, qPCR-based method. The curves were obtained by high-resolution melting analysis.

FIG. 12 Guide snoRNAs target genetic disorders caused by nonsense mutations. Schematic of the PTC-disease reporter and gsnoRNA constructs. Complementarity regions between gsnoRNAs (top) and target sites in PTC-disease gene (bottom).

FIG. 13 Complementarity regions between gsnoRNAs (top) and target sites in PTC-disease gene (bottom).

FIGS. 14A-C show that RESTART corrects nonsense mutations that can cause genetic disorders. FIGS. 14A-B, Dot plots showing the relative fraction of EGFP positive cells co-transfected with indicated gsnoRNA and RESTART v1 PTC-disease reporter constructs (FIG. 14A) and RESTART v2 DKC1-isoform3 constructs (FIG. 14B). FIG. 14C, Bar plot showing the relative fraction of EGFP positive cells co-transfected with indicated gsnoRNA and PTC-disease reporter with or without DKC1-isoform3 constructs.

FIGS. 15A-E show the delivery of RESTART by RNA oligonucleotides. FIG. 15A-C, The structures of gsnoRNAs prepared by in vitro transcription. FIG. 15D, Bar plots showing the relative fraction of EGFP positive cells transfected with the indicated gsnoRNA constructs, in vitro transcribed gsnoRNA oligonucleotides, or chemically synthesized gsnoRNA oligonucleotides. FIG. 15E, The structures of chemically synthesized gsnoRNA oligonucleotides.

### DETAILED DESCRIPTION

The present application provides methods and compositions for editing a target RNA in a host cell, comprising introducing an engineered guide small nucleolar RNA (gsnoRNA) into the host cell, wherein the gsnoRNA comprises a guide sequence that hybridizes to a sequence comprising a target uridine residue in the target RNA, and wherein the gsnoRNA recruits a DKC1 protein to modify the target uridine residue into a pseudouridine residue in the target RNA. In some aspects, the gsnoRNA is an engineered gsnoRNA comprising one or more mutations compared to a wildtype H/ACA scaffold. In some embodiments, the one or more mutations increase the editing efficiency of the gsnoRNA. In some aspects, the method further comprises increasing the cellular levels of a DKC1 protein with cytoplasmic localization, whereby the editing efficiency of the gsnoRNA/DKC1 protein complex is increased. In some aspects, the methods and compositions provided herein can be used to edit a premature termination codon (PTC) in a target gene mRNA, thereby suppressing nonsense-mediated decay of the mRNA and promoting translation of the full-length protein. In some embodiments, the methods disclosed herein can be used to treat a disease associated with a PTC in a target gene.

In some aspects, the present disclosure provides engineered gsnoRNAs and gsnoRNA scaffolds, or nucleic acid molecules encoding the gsnoRNAs. In some embodiments, the engineered gsnoRNA scaffolds are based on wildtype H/ACA snoRNA scaffolds identified by the present inventors as having higher editing efficiency compared to other scaffolds. In some embodiments, the engineered gsnoRNA scaffolds comprise mutations that increase their editing efficiency.

The methods and compositions described in the present application are based at least in part on the unexpected discovery that expression of an isoform of DKC1 with cytoplasmic localization (e.g., isoform 3 of human DKC1) significantly increases the editing efficiency of a target RNA using a gsnoRNA/DKC1 system. In one aspect, the present inventors realized that by introducing an exogenous DKC1 isoform with cytoplasmic localization, the editing efficiency of a gsnoRNA could be increased. In another aspect, the present inventors identified truncation and deletion variants of the DKC1 protein that can be used to increase the editing efficiency of a gsnoRNA.

In some aspects, provided herein are nucleic acid constructs encoding gsnoRNA for use according to the methods described herein. In some embodiments, the present inventors identified promoters and construct configurations for gsnoRNA expression that provide increased editing efficiency of the gsnoRNA.

### I. Definitions

Terms are used herein as generally used in the art, unless otherwise defined as follows.

The terms “polynucleotide,” “nucleic acid,” “nucleotide sequence,” and “nucleic acid sequence” are used interchangeably. They refer to a polymeric form of nucleotides of any length, either deoxyribonucleotides or ribonucleotides, or analogs thereof.

As used herein, “complementarity” refers to the ability of a nucleic acid to form hydrogen bond(s) with another nucleic acid by traditional Watson-Crick and Wobble base-pairing. A percent complementarity indicates the percentage of residues in a nucleic acid molecule which can form hydrogen bonds (i.e., Watson-Crick and Wobble base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence form hydrogen bonds with the same number of contiguous residues in a second nucleic acid sequence. “Substantially complementary” as used herein refers to a degree of complementarity that is at least about any one of 70%, 75%, 80%, 85%, 90%, 95%, 97%, 98%, 99%, or 100% over a region of about 40, 50, 60, 70, 80, 100, 150, 200, 250 or more nucleotides, or refers to two nucleic acids that hybridize under stringent conditions.

References to “hybridization” typically refer to specific hybridization, and exclude non-specific hybridization. Specific hybridization can occur under experimental conditions chosen, using techniques well known in the art, to ensure that the majority of stable interactions between probe and target are where the probe and target have at least 70%, preferably at least 80%, more preferably at least 90% sequence identity.

The term “mismatch” is used herein to refer to opposing nucleotides in a double stranded RNA complex which do not form perfect base pairs according to the Watson-Crick and Wobble base pairing rules. Mismatching nucleotides are G-A, C-A, U-C, A-A, G-G, C-C, U-U pairs. Wobble base pairs are: G-U, I-U, I-A, and I-C base pairs.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular, the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleic acid sequences can, alternatively, be determined using the GAP program in the GCG software package using an NWSgapdna. CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

“Percent (%) amino acid sequence identity” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5): 1792-1797, 2004; Edgar, R. C., BMC Bioinformatics 5(1): 113, 2004, each of which are incorporated herein by reference in their entirety for all purposes).

The terms “non-naturally occurring” or “engineered” are used interchangeably and indicate the involvement of the hand of man. The terms, when referring to nucleic acid molecules or polypeptides mean that the nucleic acid molecule or the polypeptide is comprises at least one modification (e.g., at least one mutation, such as a substitution, insertion, or deletion, or at least one non-naturally occurring chemical modification) compared to a naturally-occurring nucleic acid molecule or polypeptide, or is at least substantially free from at least one other component with which they are naturally associated in nature and as found in nature.

The term “wildtype” as used herein in reference to an ACA scaffold sequence refers to the sequence of a naturally occurring box H/ACA small nucleolar RNA.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to one or more ingredients in a pharmaceutical formulation, other than an active ingredient, which is nontoxic to a subject. A pharmaceutically acceptable carrier includes, but is not limited to, a buffer, excipient, stabilizer, cryoprotectant, tonicity agent, preservative, and combinations thereof. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S. Pharmacopeia or other generally recognized pharmacopeia for use in mammals, and more particularly in humans.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or condition (e.g., coronavirus infection), or a probe for specifically detecting a biomarker described herein. In certain embodiments, the manufacture or kit is promoted, distributed, or sold as a unit for performing the methods described herein.

It is understood that embodiments described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “an,” or “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Compositions and Systems

In some aspects, provided herein is an engineered gsnoRNA comprising a guide sequence that hybridizes to a sequence comprising a target uridine residue in a target RNA in a host cell, wherein the gsnoRNA comprises a nucleotide sequence selected from the group consisting of SEQ ID NOs: 4-6, 9-12, 15-19, 22-36, and 177-179, and wherein the gsnoRNA is capable of recruiting a DKC1 protein in the host cell to modify the target uridine residue into a pseudouridine residue in the target RNA. In some embodiments, the gsnoRNA comprises one or more nucleosides having 2′-OMe or 2′-MOE modifications. In some embodiments, the engineered gsnoRNA comprises no more than 10, no more than 8, no more than 6, or no more than 4 chemically modified nucleosides. In some embodiments, the engineered gsnoRNA comprises one or more phosphorothioate inter-nucleosidic linkages. In some embodiments, the gsnoRNA comprises no more than 10, no more than 9, no more than 8, or no more than 6 phosphorothioate inter-nucleosidic linkages. In some embodiments, the gsnoRNA comprises a 5′ cap modification (e.g., a 7-methylguanosine (m⁷G) cap modification). In some embodiments, the 5′ cap modification is introduced by in vitro transcription using an m⁷G(5′)ppp(5′)G cap analog.

In some embodiments, the engineered gsnoRNA is produced by in vitro transcription. In some embodiments, the engineered gsnoRNA produced by in vitro transcription is a full-length gsnoRNA (e.g., comprising a 3′ hairpin, a 5′ hairpin, an H box, and an ACA box). In some embodiments, the engineered gsnoRNA produced by in vitro transcription comprises a 5′ cap modification (e.g., a 7-methylguanosine (m⁷G) cap modification).

In some embodiments, the engineered gsnoRNA comprises a single hairpin and an H box, but does not comprise an ACA box. In some embodiments, the engineered gsnoRNA comprises the sequence of SEQ ID NO: 179. In some embodiments, the engineered gsnoRNA comprises a single hairpin and an ACA box, but does not comprise an H box. In some embodiments, the engineered gsnoRNA comprises the sequence of SEQ ID NO: 180. In some embodiments, the gsnoRNA comprises one or more nucleosides having 2′-OMe or 2′-MOE modifications. In some embodiments, the engineered gsnoRNA comprises no more than 10, no more than 8, no more than 6, or no more than 4 chemically modified nucleosides. In some embodiments, the engineered gsnoRNA comprises one or more phosphorothioate inter-nucleosidic linkages. In some embodiments, the gsnoRNA comprises no more than 10, no more than 9, no more than 8, or no more than 6 phosphorothioate inter-nucleosidic linkages.

In some aspects, provided herein is an engineered gsnoRNA comprising a guide sequence that hybridizes to a sequence comprising a target uridine residue in a target RNA in a host cell, wherein the gsnoRNA comprises a scaffold sequence derived from wildtype ACA2b or ACA36, and wherein the gsnoRNA is capable of recruiting a DKC1 protein in the host cell to modify the target uridine residue into a pseudouridine residue in the target RNA. In some embodiments, the gsnoRNA comprises a scaffold sequence derived from the sequence of SEQ ID NOs: 11 or 12. In some embodiments, the gsnoRNA comprises one, two, three, or four substitution, deletion, and/or insertion mutations compared to SEQ ID NOs: 11 or 12. In some embodiments, the gsnoRNA comprises one or more nucleosides having 2′-OMe or 2′-MOE modifications. In some embodiments, the engineered gsnoRNA comprises no more than 10, no more than 8, no more than 6, or no more than 4 chemically modified nucleosides. In some embodiments, the engineered gsnoRNA comprises one or more phosphorothioate inter-nucleosidic linkages. In some embodiments, the gsnoRNA comprises no more than 10, no more than 9, no more than 8, or no more than 6 phosphorothioate inter-nucleosidic linkages. In some embodiments, the gsnoRNA comprises a 5′ cap modification (e.g., a 7-methylguanosine (m⁷G) cap modification). In some embodiments, the 5′ cap modification is introduced by in vitro transcription using an m⁷G(5′)ppp(5′)G cap analog.

In some aspects, provided herein is an isolated nucleic acid molecule comprising a sequence encoding a gsnoRNA provided herein. In some embodiments, the gsnoRNA comprises a scaffold sequence derived from a wildtype H/ACA-snoRNA selected from the group consisting of ACA19, ACA2b, ACA36, ACA44, ACA27, E2, ACA3, and ACA17. In some embodiments, the gsnoRNA comprises a scaffold sequence derived from a wildtype H/ACA-snoRNA selected from the group consisting of ACA2b, ACA36, ACA44, ACA27, E2, ACA3, and ACA17. In some embodiments, the gsnoRNA comprises a scaffold sequence derived from ACA36. In some embodiments, the gsnoRNA comprises a scaffold sequence derived from ACA2b. In some embodiments, the gsnoRNA comprises a scaffold sequence derived from ACA19. In some embodiments, the nucleic acid molecule further comprises a sequence encoding an agent that promotes expression of isoform 3 of a DKC1 protein (e.g., a splice-switching antisense oligonucleotide (ASO), wherein the ASO enhances expression of a DKC1 protein that is an endogenous DKC1 isoform with cytoplasmic localization in the host cell). In some embodiments, the nucleic acid molecule further comprises a sequence encoding a DKC1 isoform or DKC1 protein variant, wherein the isoform or variant has cytoplasmic localization. Exemplary DKC1 proteins are described in Section II A below.

In some aspects, provided herein is an engineered RNA-editing system comprising: (a) a gsnoRNA (such as any one of the gsnoRNAs described in Section II B below) comprising a guide sequence that hybridizes to a sequence comprising a target uridine residue in a target RNA in a host cell, or a nucleic acid molecule encoding the gsnoRNA; and (b) a DKC1 protein (such as any one of the DKC1 proteins described in Section II A below), or a nucleic acid molecule encoding the DKC1 protein, wherein the gsnoRNA is capable of recruiting the DKC1 protein to modify the target uridine residue into a pseudouridine residue in the target RNA. In some embodiments, the DKC1 protein is a DKC1 isoform with cytoplasmic localization.

In some aspects, provided herein is a host cell comprising any of the gsnoRNAs, nucleic acid constructs/molecules, or engineered RNA-editing systems described herein.

In some aspects, provided herein is a kit for editing a target RNA in a host cell, comprising any of the gsnoRNAs, nucleic acid constructs/molecules, or engineered RNA-editing systems described herein.

### A. DKC1 Protein

The present application in some embodiments provides engineered DKC1 proteins or nucleic acid constructs encoding a DKC1 protein.

Dyskerin (DKC1) is a highly conserved multifunctional protein that acts as RNA-guided pseudouridine synthase, directing the enzymatic conversion of specific uridines to pseudouridines. It concentrates in the nucleoli and the Cajal bodies (CBs) where, in association with three other highly conserved proteins (Nop10, Nhp2, Gar1), DKC1 composes a tetramer able to enter in the composition of different nuclear RNPs playing key biological functions. Within the nucleolus, the tetramer associates with H/ACA small nucleolar RNAs (snoRNAs) to compose the H/ACA snoRNPs, that regulate rRNA processing and pseudouridylate RNA targets by snoRNA-guided base complementarity. Within the CBs, it associates with CB specific small RNAs (scaRNAs) to compose the scaRNPs, that direct pseudouridylation of spliceosomal snoRNAs.

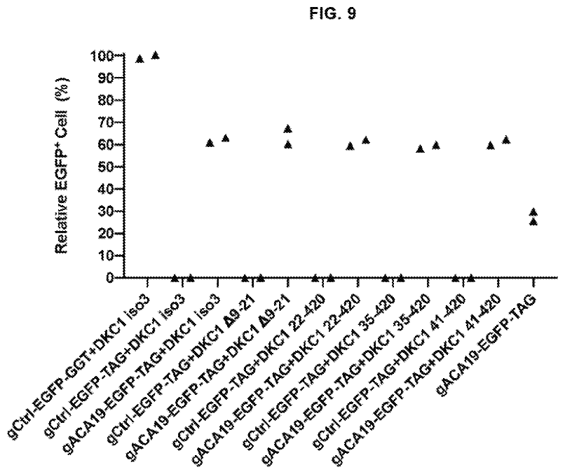

There are two DKC1 isoforms in human cells: DKC1 isoform 1 is the canonical DKC1 form containing the bipartite N- and C-terminal nuclear localization signals (NLSs); DKC1 isoform 3 is an alternative splicing variant, which is produced by retention of the intron 12 and lacks C-terminal NLS (FIG. 9A). The endogenous mRNA expression level of isoform1 is approximately 20-fold greater than that of isoform 3⁵. Surprisingly, the present inventors found that increasing the level of DKC1 isoform 3 enhances the target pseudouridylation editing efficiency (e.g., editing efficiency of target mRNAs) guided by gsnoRNAs.

In some aspects, compositions of the present disclosure comprise nucleic acid constructs for expression of a DKC1 protein. In some aspects, compositions of the present disclosure comprise a DKC1 protein (e.g., a DKC1 protein in complex with a gsnoRNA). In some embodiments, the DKC1 protein is isoform 3 of a mammalian DKC1 protein. In some embodiments, the DKC1 protein is homologous to isoform 3 of a human DKC1 protein. In some embodiments, the DKC1 protein has at least 75%, 80%, 85%, 90%, 95%, 96%, 97%, 98%, or 99% sequence identity to isoform 3 of human DKC1 protein. In some embodiments, the DKC1 protein is isoform 3 of human DKC1 protein. In some embodiments, the DKC1 protein comprises an amino acid sequence having at least 85% (e.g., at least any of 90%, 95%, 96%, 97%, 98%, 99%, or 100%) sequence identity to SEQ ID NO: 2. The sequence of full-length DKC1 (isoform 1) and isoform 3 DKC1 are shown in Table 1 below.

In some embodiments, the DKC1 protein is part of a ribonucleoprotein (RNP) complex that associates with the gsnoRNA. In some embodiments, the ribonucleoprotein complex comprises NOP10, GAR1, and/or NHP2.

In some aspects, provided herein are truncated DKC1 protein variants and nucleic acid constructs encoding the same. In some embodiments, the DKC1 protein comprises a deletion of a nuclear localization signal (NLS) relative to a wildtype DKC1 protein of the same species. In some embodiments, the DKC1 protein comprises a deletion of amino acid residues 9-21 of DKC1 isoform 3, wherein the amino acid numbering is based on SEQ ID NO: 2. In some embodiments, the DKC1 protein comprises amino acid residues 22-420 of DKC1 isoform 3, wherein the amino acid numbering is based on SEQ ID NO: 2. In some embodiments, the DKC1 protein comprises amino acid residues 35-420 of DKC1 isoform 3, wherein the amino acid numbering is based on SEQ ID NO: 2. In some embodiments, the DKC1 protein comprises amino acid residues 41-420 of DKC1 isoform 3, wherein the amino acid numbering is based on SEQ ID NO: 2. Although the DKC1 sequence in SEQ ID NO: 2 is isoform 3 of human DKC1, the person of ordinary skill in the art will understand how to generate corresponding truncation and deletion variants of homologous DKC1 proteins based on sequence alignments (e.g., corresponding deletion/truncation variants of DKC1 proteins from other mammalian species).

In some embodiments, the DKC1 protein comprises an amino acid sequence having at least 85% (e.g., at least any of 90%, 95%, 96%, 97%, 98%, 99%, or 100%) sequence identity to SEQ ID NO: 85. In some embodiments, the DKC1 protein comprises an amino acid sequence having at least 85% (e.g., at least any of 90%, 95%, 96%, 97%, 98%, 99%, or 100%) sequence identity to SEQ ID NO: 86. In some embodiments, the DKC1 protein comprises an amino acid sequence having at least 85% (e.g., at least any of 90%, 95%, 96%, 97%, 98%, 99%, or 100%) sequence identity to SEQ ID NO: 87. In some embodiments, the DKC1 protein comprises an amino acid sequence having at least 85% (e.g., at least any of 90%, 95%, 96%, 97%, 98%, 99%, or 100%) sequence identity to SEQ ID NO: 88.

TABLE 1
DKC1 protein sequences

| DKC1 | | SEQ ID |
|---|---|---|
| protein | Sequence | NO. |
| full-length | MADAEVIILPKKHKKKKERKSLPEEDVAEIQHAEEFLIKPESKVAKLDTSQWPL | 1 |
| human | LLKNFDKLNVRITHYTPLACGSNPLKREIGDYIRTGFINLDKPSNPSSHEVVAW | |
| DKC1 | IRRILRVEKTGHSGTLDPKVTGCLIVCIERATRLVKSQQSAGKEYVGIVRLHNA | |
| protein | IEGGTQLSRALETLTGALFQRPPLIAAVKRQLRVRTIYESKMIEYDPERRLGIF | |
| | WVSCEAGTYIRTLCVHLGLLLGVGGQMQELRRVRSGVMSEKDHMVTMHDVLDAQ | |
| | WLYDNHKDESYLRRVVYPLEKLLTSHKRLVMKDSAVNAICYGAKIMLPGVLRYE | |
| | DGIEVNQEIVVITTKGEAICMAIALMTTAVISTCDHGIVAKIKRVIMERDTYPR | |
| | KWGLGPKASQKKLMIKQGLLDKHGKPTDSTPATWKQEYVDYSESAKKEVVAEVV | |
| | KAPQVVAEAAKTAKRKRESESESDETPPAAPQLIKKEKKKSKKDKKAKAGLESG | |
| | AEPGDGDSDTTKKKKKKKKAKEVELVSE | |
| human | MADAEVIILPKKHKKKKERKSLPEEDVAEIQHAEEFLIKPESKVAKLDTSQWPL | 2 |
| DKC1 | LLKNFDKLNVRITHYTPLACGSNPLKREIGDYIRIGFINLDKPSNPSSHEVVAW | |
| isoform 3 | IRRILRVEKTGHSGTLDPKVTGCLIVCIERATRLVKSQQSAGKEYVGIVRLHNA | |
| | IEGGTQLSRALETLTGALFQRPPLIAAVKRQLRVRTIYESKMIEYDPERRLGIF | |
| | WVSCEAGTYIRTLCVHLGLLLGVGGQMQELRRVRSGVMSEKDHMVTMHDVLDAQ | |
| | WLYDNHKDESYLRRVVYPLEKLLISHKRLVMKDSAVNAICYGAKIMLPGVLRYE | |
| | DGIEVNQEIVVITTKGEAICMAIALMTTAVISTCDHGIVAKIKRVIMERDTYPR | |
| | KWGLGPKASQKKLMIKQGLLDKHGKPTDSTPATWKQEYVDYR | |
| Amino | MADAEVIILPKKHKKKKERKSLPEEDVAEIQHAEEFLIKPESKVAKLDTSQWPL | 85 |
| acids 1-419 | LLKNEDKLNVRITHYTPLACGSNPLKREIGDYIRTGFINLDKPSNPSSHEVVAW | |
| of human | IRRILRVEKTGHSGTLDPKVTGCLIVCIERATRLVKSQQSAGKEYVGIVRLHNA | |
| DKC1 | IEGGTQLSRALETLTGALFQRPPLIAAVKRQLRVRTIYESKMIEYDPERRLGIF | |
| | WVSCEAGTYIRTLCVHLGLLLGVGGQMQELRRVRSGVMSEKDHMVTMHDVLDAQ | |
| | WLYDNHKDESYLRRVVYPLEKLLTSHKRLVMKDSAVNAICYGAKIMLPGVLRYE | |
| | DGIEVNQEIVVITTKGEAICMAIALMTTAVISTCDHGIVAKIKRVIMERDTYPR | |
| | KWGLGPKASQKKLMIKQGLLDKHGKPTDSTPATWKQEYVDY | |
| human | MADAEVIILPEEDVAEIQHAEEFLIKPESKVAKLDTSQWPLLLKNEDKLNVRTT | 86 |
| DKC1 | HYTPLACGSNPLKREIGDYIRTGFINLDKPSNPSSHEVVAWIRRILRVEKTGHS | |
| isoform 3 | GTLDPKVTGCLIVCIERATRLVKSQQSAGKEYVGIVRLHNAIEGGTQLSRALET | |
| 49-21 | LTGALFQRPPLIAAVKRQLRVRTIYESKMIEYDPERRLGIFWVSCEAGTYIRTL | |
| | CVHLGLLLGVGGQMQELRRVRSGVMSEKDHMVTMHDVLDAQWLYDNHKDESYLR | |
| | RVVYPLEKLLTSHKRLVMKDSAVNAICYGAKIMLPGVLRYEDGIEVNQEIVVIT | |
| | TKGEAICMAIALMTTAVISTCDHGIVAKIKRVIMERDTYPRKWGLGPKASQKKL | |
| | MIKQGLLDKHGKPTDSTPATWKQEYVDYR | |
| Truncation | MLPEEDVAEIQHAEEFLIKPESKVAKLDTSQWPLLLKNEDKLNVRTTHYTPLAC | 13 |
| 22-420 of | GSNPLKREIGDYIRTGFINLDKPSNPSSHEVVAWIRRILRVEKTGHSGTLDPKV | |
| human | TGCLIVCIERATRLVKSQQSAGKEYVGIVRLHNAIEGGTQLSRALETLTGALFQ | |
| DKC1 | RPPLIAAVKRQLRVRTIYESKMIEYDPERRLGIFWVSCEAGTYIRTLCVHLGLL | |
| isoform 3 | LGVGGQMQELRRVRSGVMSEKDHMVTMHDVLDAQWLYDNHKDESYLRRVVYPLE | |
| | KLLTSHKRLVMKDSAVNAICYGAKIMLPGVLRYEDGIEVNQEIVVITTKGEAIC | |
| | MAIALMTTAVISTCDHGIVAKIKRVIMERDTYPRKWGLGPKASQKKLMIKQGLL | |
| | DKHGKPTDSTPATWKQEYVDYR | |
| Truncation | MEFLIKPESKVAKLDTSQWPLLLKNEDKLNVRTTHYTPLACGSNPLKREIGDYI | 87 |
| 35-420 of | RTGFINLDKPSNPSSHEVVAWIRRILRVEKTGHSGTLDPKVIGCLIVCIERATR | |
| human | LVKSQQSAGKEYVGIVRLHNAIEGGTQLSRALETLTGALFQRPPLIAAVKRQLR | |
| DKC1 | VRTIYESKMIEYDPERRLGIFWVSCEAGTYIRTLCVHLGLLLGVGGQMQELRRV | |
| isoform 3 | RSGVMSEKDHMVTMHDVLDAQWLYDNHKDESYLRRVVYPLEKLLTSHKRLVMKD | |
| | SAVNAICYGAKIMLPGVLRYEDGIEVNQEIVVITTKGEAICMAIALMTTAVIST | |
| | CDHGIVAKIKRVIMERDTYPRKWGLGPKASQKKLMIKQGLLDKHGKPTDSTPAT | |
| | WKQEYVDYR | |
| Truncation | MESKVAKLDTSQWPLLLKNFDKLNVRTTHYTPLACGSNPLKREIGDYIRTGFIN | 88 |
| 41-420 of | LDKPSNPSSHEVVAWIRRILRVEKTGHSGTLDPKVTGCLIVCIERATRLVKSQQ | |
| DKC1 | SAGKEYVGIVRLHNAIEGGTQLSRALETLTGALFQRPPLIAAVKRQLRVRTIYE | |
| isoform 3 | SKMIEYDPERRLGIFWVSCEAGTYIRTLCVHLGLLLGVGGQMQELRRVRSGVMS | |
| | EKDHMVTMHDVLDAQWLYDNHKDESYLRRVVYPLEKLLTSHKRLVMKDSAVNAI | |
| | CYGAKIMLPGVLRYEDGIEVNQEIVVITTKGEAICMAIALMTTAVISTCDHGIV | |
| | AKIKRVIMERDTYPRKWGLGPKASQKKLMIKQGLLDKHGKPTDSTPATWKQEYV | |
| | DYR | |

In some embodiments, amino acid sequence variants of the DKC1 proteins provided herein are contemplated. For example, it may be desirable to improve the stability and/or other biological properties of DKC1 (e.g., of the catalytic domain of DKC1) or of its interaction with other proteins in a ribonucleoprotein complex. Structures of DKC1 and other proteins in the ribonucleoprotein complex have been described, for example in Rashid et al. (Molecular Cell (2006) 21(2): 249-260) and Czekay et al. (Front. Microbiol. (2021) 12:654370), the contents of which are herein incorporated by reference in their entirety. Amino acid sequence variants of a DKC1 protein may be prepared by introducing appropriate modifications into the nucleotide sequence encoding the target-binding moiety, or by peptide synthesis. Such modifications include, for example, deletions from, and/or insertions into and/or substitutions of residues within the amino acid sequences of the target-binding moiety. Any combination of deletion, insertion, and substitution can be made to arrive at the final construct, provided that the final construct possesses the desired characteristics.

In some embodiments, DKC1 protein variants having one or more amino acid substitutions are provided. Amino acid substitutions may be introduced into a DKC1 protein and the products screened for a desired activity

Conservative substitutions are shown in Table A below.

TABLE A
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

Also contemplated are fusion proteins comprising a fragment of a naturally occurring DKC1 protein or a functional variant thereof and a heterologous amino acid sequence, e.g., at the N-terminus, the C-terminus, or an internal location of the DKC1 fragment.

B. Nucleic Acid Constructs and Engineered gsnoRNA

In some aspects, provided herein are engineered gsnoRNA based on H/ACA snoRNAs. In some embodiments, the gsnoRNA comprises a single guide sequence. In some embodiments, the gsnoRNA comprise two guide sequences. In some embodiments, the engineered gsnoRNA comprises more than two (e.g., 3, 4, 5, 6, or more) guide sequences. For example, H/ACA snoRNAs contain two hairpins followed by the Hand ACA box motifs. In some embodiments, both hairpins of the engineered gsnoRNAs provided herein contain guide sequences that are capable of targeting the target pseudouridylation site. In other embodiments, only one hairpin of an engineered gsnoRNA contains a guide sequence capable of targeting the target pseudouridylation site. Exemplary engineered gsnoRNA sequences are provided in Tables 2 and 3 below.

In some aspects, gsnoRNAs disclosed herein are synthetic oligonucleotides, which can be synthesized according to methods known in the art. In some embodiments, gsnoRNAs according to the present disclosure are oligoribonucleotides (full RNA). However, in some embodiments, gsnoRNAs of the present disclosure may comprise DNA. In some embodiments, especially when exclusively consisting of nucleotides or linkages that can be expressed in a biological system, gsnoRNAs may be expressed in situ, e.g. from a plasmid or a viral vector.

In some aspects, the gsnoRNA comprises a scaffold sequence derived from a wildtype H/ACA-snoRNA selected from the group consisting of ACA2b and ACA36. In some aspects, the editing efficiency of a gsnoRNA derived from a wildtype H/ACA scaffold is at least 5% (e.g., between or between about 5%-15% or 5-10%) in mammalian cells (e.g., in human cells such as HEK293T cells). In some embodiments, the gsnoRNA comprises a scaffold sequence derived from ACA2b. In some embodiments, the gsnoRNA comprises a scaffold sequence derived from ACA36. In some embodiments, the gsnoRNA comprises a scaffold sequence derived from ACA19.

In some aspects, disclosed herein are engineered gsnoRNA and engineered gsnoRNA scaffolds derived from wildtype H/ACA-snoRNA (e.g., from ACA2b, ACA36, or ACA19), wherein the gsnoRNA are capable of modifying a PTC in an RNA encoding a protein, wherein said modification results in expression of the full-length protein. In some embodiments, the engineered gsnoRNA is capable of causing expression of the full-length protein in the host cell of at least 4% (e.g., at least 5%, at least 6%, at least 7%, at least 8%, at least 9% or at least 10%) of the expression level of the full-length protein without a premature termination codon. In some embodiments, the engineered gsnoRNA is capable of causing expression of the full-length protein, wherein the expression of the protein is detectable without enrichment (e.g., without enrichment by immunoprecipitation). In some embodiments, the protein is detected via a tag (e.g., via a fluorescent tag). In some embodiments, the protein is detected by immo-staining according to methods known in the art. In some embodiments, the engineered gsnoRNA is capable of causing expression of the full-length protein in at least 20% of host cells (e.g., at least 25%, at least 30%, at least 35%, at least 40%, at least 45%, or at least 50% of host cells).

In some embodiments, the gsnoRNA comprises one or more guide sequences each located in a region corresponding to a hairpin structure of the wildtype H/ACA-snoRNA. In some embodiments, the gsnoRNA comprise one or more guide sequences located in a hairpin structure at the 3′ terminal part of the wildtype H/ACA-snoRNA. In some embodiments, the gsnoRNA comprise one or more guide sequences located in a hairpin structure at the S′ terminal part of the wildtype H/ACA-snoRNA.

In some embodiments, the gsnoRNA comprises one or more mutations (e.g., substitution, insertion and/or deletion) in one or more hairpin structures (e.g., the 3′ and/or 5′ hairpin structures) of the wildtype ACA19. In some embodiments, the gsnoRNA comprises one or more mutations that alter the distance between the nucleotide residue in the guide region that hybridizes to the target uridine and the H/ACA box compared to a wildtype scaffold. In some embodiments, the one or more mutations comprise insertion or deletion of one or more nucleotide residues. In some embodiments, the engineered gsnoRNA comprises 14 nucleotides between the nucleotide residue in the guide region that hybridizes to the target uridine and the H/ACA box. In some embodiments, the engineered gsnoRNA comprises 15 nucleotides between the nucleotide residue in the guide region that hybridizes to the target uridine and the H/ACA box. In some embodiments, said mutations increase the efficiency of pseudouridylation (e.g., the efficiency of PTC-readthrough) by at least 1.2-, 1.3-, 1.4-, 1.5-, or 1.6-fold compared to the wildtype scaffold.

In some embodiments, the one or more mutations comprise substitutions in a small polyU sequence (e.g., a sequence of 4 or more, or 5 or more consecutive uridine (U) residues). In some embodiments, the one or more mutations comprise altering a small polyU sequence so that it comprises no more than two consecutive U residues. In some embodiments, the one or more mutations comprise a single base mutation in a “UUUU” sequence. In some embodiments, the mutation is a “UUCU” mutation or a “UGUU” mutation. In some embodiments, the mutated polyU sequence is located in a loop region of the gsnoRNA scaffold. In some embodiments, the engineered gsnoRNA comprises the sequence of SEQ ID NO. 49 or 50. In some embodiments, the engineered gsnoRNA comprises the sequence of SEQ ID NO. 15 or 16. In some embodiments, said mutations increase the efficiency of pseudouridylation (e.g., the efficiency of PTC-readthrough) by at least 1.2-, 1.3-, 1.4-, 1.5-, or 1.6-fold compared to the wildtype scaffold.

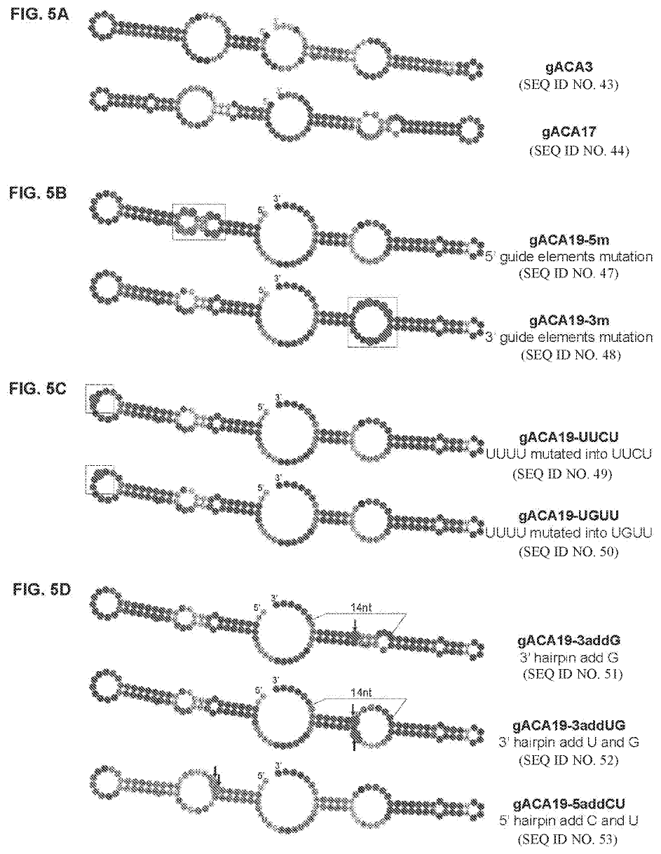

In some embodiments, the one or more mutations comprise mutations that increase the openness of a guide region compared to the guide region of a wildtype scaffold. In some embodiments, the one or more mutations reduce the base-pairing probability of one or more residues within a guide region of the gsnoRNA scaffold (e.g., the 5′ guide region of the gACA19 scaffold). In some embodiments, the one or more mutations comprise insertion or one or more nucleotides. In some embodiments, the one or more mutations comprise the addition of CU after residue 8, wherein the numbering is according to SEQ ID NO: 37. In some embodiments, the engineered gsnoRNA is the gsnoRNA of SEQ ID NO: 53. The predicted secondary structure of gACA19-5addCU (SEQ ID NO: 53) is shown in FIG. 5D. In some embodiments, said mutations increase the efficiency of pseudouridylation (e.g., the efficiency of PTC-readthrough) by at least 1.2-, 1.3-, 1.4-, 1.5-, or 1.6-fold compared to the wildtype scaffold.

In some embodiments, the one or more mutations are selected from the group consisting of substitution of residues 26-29 with UUCU, substitution of residues 26-29 with UGUU, addition of G to the 3′ hairpin structure after residue 115, and addition of a dinucleotide sequence (XX, e.g., CU) to the 5′ hairpin after residue 8, wherein X is a nucleotide selected from A, U, C, and G, and wherein the numbering is according to SEQ ID NO: 37.

In some embodiments, gsnoRNA comprises a nucleotide sequence selected from the group consisting of SEQ ID NOs: 17-19 and 22-29.

In some embodiments, the gsnoRNA comprises a nucleotide sequence selected from the group consisting of SEQ ID NOs: 4-6, 9-12, 15-19, 22-36, and 177-179.

In some embodiments, the gsnoRNA comprises a nucleotide sequence selected from the group consisting of SEQ ID NOs: 15-19.

In some embodiments, the gsoRNA comprises a nucleotide sequence selected from the group consisting of SEQ ID NOs: 20-21 and 145-150.

In some embodiments, the gsnoRNA is a disease-targeting gsnoRNA (e.g., any of the gsnoRNA sequences provided in Table 4).

In some embodiments, the gsnoRNA comprises one or more chemically modified nucleosides and/or inter-nucleosidic linkages. In some embodiments, the gsnoRNA comprises one or more nucleosides having 2′ O-methyl (2′-OMe) or 2′-O-methoxyethyl (2′-MOE) modifications. In some embodiments, a gsnoRNA according to the present disclosure may be chemically modified almost in its entirety, for example by providing nucleotides with a 2′-O-methylated sugar moiety (2′-OMe) and/or with a 2′-O-methoxyethyl sugar moiety (2′-MOE). In some embodiments, the gsnoRNA comprises no more than 20, no more than 15, no more than 10, no more than 8, no more than 6, or no more than 4 2′-OMe or 2′-MOE modifications. In some embodiments, the gsnoRNA comprises between about 2 and about 6 2′-OMe or 2′-MOE modifications. In some embodiments, the gsnoRNA comprises about 4 2′-OMe or 2′-MOE modifications. In some embodiments, the gsnoRNA comprises no more than 5 modified sugars. In some embodiments, the gsnoRNA comprises two nucleosides comprising modified sugar moieties (e.g., 2′-OMe) at the 5′ end and two nucleosides comprising modified sugar moieties (e.g., 2′-OMe) at the 3′ end of the gsnoRNA. In some embodiments, the gsnoRNA comprises no more than four, three, or two nucleosides comprising modified sugar moieties (e.g., 2′-OMe) at the 5′ end and no more than four, three, or two nucleosides comprising modified sugar moieties (e.g., 2′-OMe) at the 3′ end of the gsnoRNA. In some embodiments, the gsnoRNA comprises one or more phosphorothioate inter-nucleosidic linkages. In some embodiments, the gsnoRNA comprises no more than 20, no more than 15, no more than 10, no more than 8, or no more than 6 phosphorothioate linkages. In some embodiments, the gsnoRNA comprises between about 2 and about 10 phosphorothioate linkages. In some embodiments, the gsnoRNA comprises about 6 phosphorothioate linkages. In some embodiments, the gsnoRNA comprises about three phosphorothioate linkages at the 5′ end and about three phosphorothioate linkages at the 3′ end of the gsnoRNA. In some embodiments, the gsnoRNA comprises no more than five, four, or three phosphorothioate linkages at the 5′ end and no more than five, four, or three phosphorothioate linkages at the 3′ end of the gsnoRNA. Example 7 provides results demonstrating that a limited number of modifications is sufficient for stability and function of gsnoRNA oligonucleotides.

In some embodiments, the gsnoRNA comprises one or more chemically modified nucleosides and/or inter-nucleosidic linkages. In some embodiments, the gsnoRNA comprises one or more nucleosides having 2′ O-methyl (2′-OMe) or 2′-O-methoxyethyl (2′-MOE) modifications. In some embodiments, a gsnoRNA according to the present disclosure may be chemically modified almost in its entirety, for example by providing nucleotides with a 2′-O-methylated sugar moiety (2′-OMe) and/or with a 2′-O-methoxyethyl sugar moiety (2′-MOE). In some embodiments In some embodiments, the gsnoRNA comprises a 5′ hairpin, an H box (consensus sequence ANANNA), a 3′ hairpin, and an ACA box (consensus sequence ANA). In some embodiments, the gsnoRNA comprises a single hairpin and an H box (referred to herein as a gH5 or rH5 for 5′ half gsnoRNA encoding sequence or gsnoRNA oligonucleotide, respectively), and lacks an ACA box. In some embodiments, the gsnoRNA comprises a single hairpin and an ACA box (referred to herein as a gH3 or rH3 for 3′ half gsnoRNA encoding sequence or gsnoRNA oligonucleotide, respectively), and lacks an H box. In some embodiments, the gsnoRNA comprising a single hairpin is between 60 and 70 nucleotides in length. In some embodiments, the gsnoRNA comprising a single hairpin is about 65 nucleotides in length.

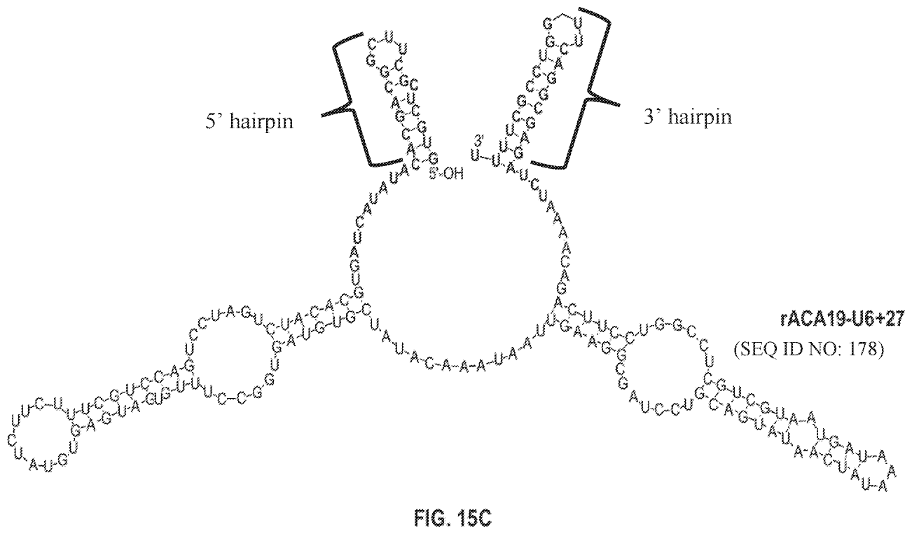

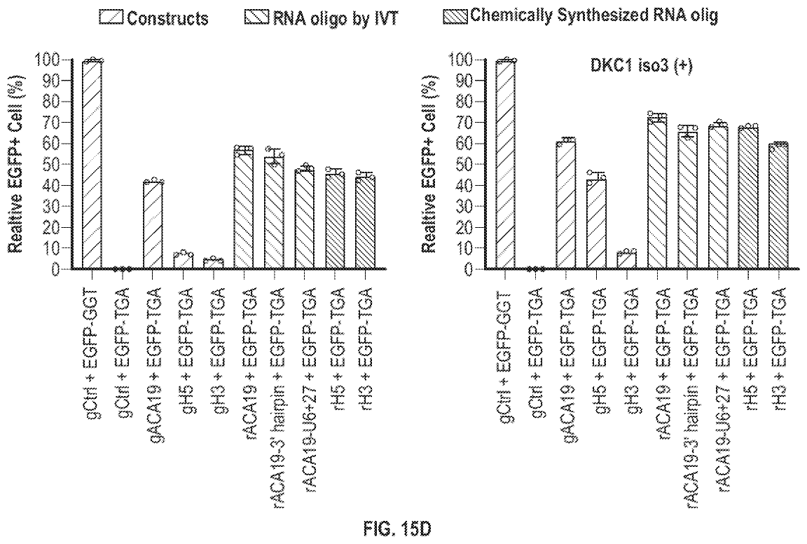

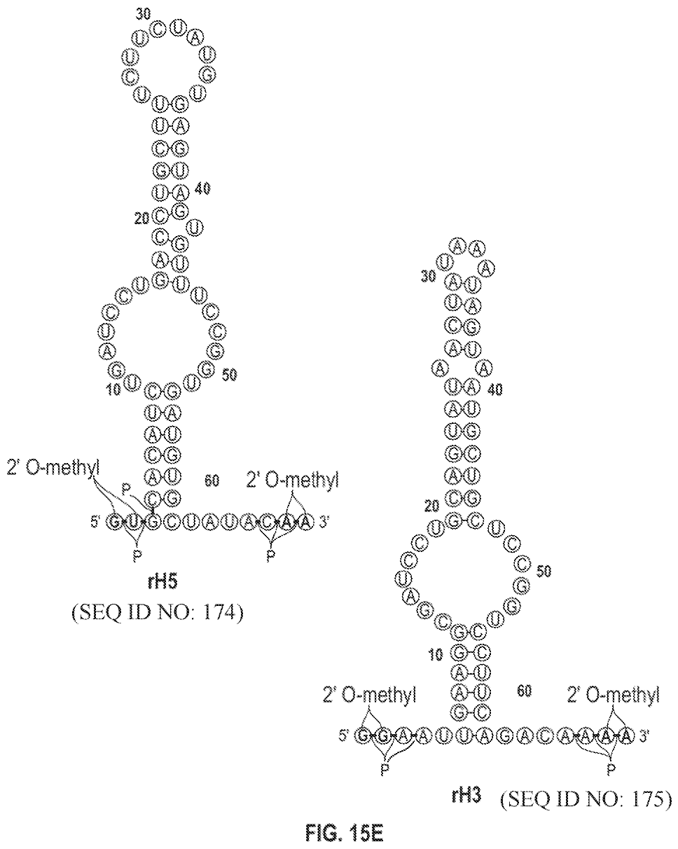

In some embodiments, the gsnoRNA is prepared by in vitro transcription. In some embodiments, the gsnoRNA prepared by in vitro transcription comprises the sequence of any one of SEQ ID NOs: 4-6, 9-12, 15-19, 22-36. In some embodiments, the gsnoRNA prepared by in vitro transcription comprises a 5′ cap modification or a 5′ hairpin (e.g., of a U6+U27 expression cassette). In some embodiments, the gsnoRNA prepared by in vitro transcription comprises a 5′ cap modification. In some embodiments, the 5′ cap modification is is a m⁷G modification (e.g., a cap 0, cap 1, or cap 2 modification) or an m⁶Aₘ modification. Suitable methods for adding a 5′ cap to an RNA oligonucleotide have been described, for example, in U.S. Pat. No. 10,494,399, the contents of which are herein incorporated by reference in their entirety. In some embodiments, the gsnoRNA further comprises a 3′ hairpin (e.g., the gsnoRNA comprises the sequence of any one of SEQ ID NOs: 4-6, 9-12, 15-19 and 22-36 and a 3′ hairpin). In some embodiments, the gsnoRNA comprises a 5′ cap modification and does not comprise a 3′ hairpin (e.g., as shown in FIG. 15A). In some embodiments, the 5′ cap modification is introduced by in vitro transcription using an m⁷G(5′)ppp(5′)G cap analog.

Various chemistries and modification are known in the field of oligonucleotides that can be readily used in accordance with the disclosure. The regular internucleosidic linkages between the nucleotides may be altered by mono- or di-thioation of the phosphodiester bonds to yield phosphorothioate esters or phosphorodithioate esters, respectively. Other modifications of the internucleosidic linkages are possible, including amidation and peptide linkers. In a preferred aspect the gsnoRNAs of the present disclosure have one, two, three, four, five, six or more phosphorothioate linkages between the most terminal nucleotides of the gsnoRNA (hence, preferably at both the 5′ and 3′ end), which means that in the case of three phosphorothioate linkages, the ultimate four nucleotides are linked accordingly. It will be understood by the skilled person that the number of such linkages may vary on each end, depending on the target sequence, or based on other aspects, such as toxicity. However, it is some embodiments of the disclosure that the gsnoRNA does comprise one or more PS linkages between any position at its terminal seven nucleotides.

The ribose sugar may be modified by substitution of the 2′-0 moiety with a lower alkyl (C1-4, such as 2′-OMe), alkenyl (C2-4), alkynyl (C2-4), methoxyethyl (2′-methoxyethoxy; or 2′-O-methoxyethyl; or 2′-MOE), or other substituent. In some embodiments, substituents of the 2′ OH group are a methyl, methoxyethyl or 3,3′-dimethylallyl group. The latter is known for its property to inhibit nuclease sensitivity due to its bulkiness, while improving efficiency of hybridization. Alternatively, locked nucleic acid sequences (LNAs), comprising a 2′-4′ intramolecular bridge (usually a methylene bridge between the 2′ oxygen and 4′ carbon) linkage inside the ribose ring, may be applied. Purine nucleobases and/or pyrimidine nucleobases may be modified to alter their properties, for example by amination or deamination of the heterocyclic rings. Other modifications that may be present in the gsnoRNAs of the present disclosure are 2′-F modified sugars, BNA and cEt. The exact chemistries and formats may depend from oligonucleotide construct to oligonucleotide construct and from application to application, and may be worked out in accordance with the wishes and preferences of those of skill in the art.

Examples of chemical modifications in the gsnoRNAs of the present disclosure are modifications of the sugar moiety, including by cross-linking substituents within the sugar (ribose) moiety (e.g. as in LNA or locked nucleic acids, BNA, cEt and the like), by substitution of the 2′-O atom with alkyl (e.g. 2′-O-methyl), alkynyl (2′-O-alkynyl), alkenyl (2′- O)-alkenyl), alkoxyalkyl (e.g. 2′-O-methoxyethyl, 2′-MOE) groups, having a length as specified above, and the like. In the context of the present disclosure, a sugar ‘modification’ also comprises 2′ deoxyribose (as in DNA). In addition, the phosphodiester group of the backbone may be modified by thioation, dithioation, amidation and the like to yield phosphorothioate, phosphorodithioate, phosphoramidate, etc., internucleosidic linkages. The internucleosidic linkages may be replaced in full or in part by peptidic linkages to yield in peptidonucleic acid sequences and the like. Alternatively, or in addition, the nucleobases may be modified by (de)amination, to yield inosine or 2′6′-diaminopurines and the like. A further modification may be methylation of the C5 in the cytidine moiety of the nucleotide, to reduce potential immunogenic properties known to be associated with CpG sequences.

In some embodiments, the gsnoRNA does not comprise one or more chemically modified nucleosides and/or inter-nucleosidic linkages. In some embodiments, the gsnoRNA does not comprise any non-natural inter-nucleosidic linkages.

Mammalian H/ACA snoRNAs are generally embedded (positioned) within pre-mRNA intronic regions of protein-coding genes. During transcription elongation, several proteins with a functional role in pseudouridylation, such as NOP10, dyskerin (DKC1) or NHP2 bind to the nascent H/ACA snoRNA sequences. Following splicing, the guide RNAs are processed through debranching and exonucleolytic processing, resulting in a RNA-protein complex called ‘small nuclear ribonucleoproteins’ (snRNPs, or snRNP complex). Box H/ACA snoRNAs have no preference for localization relative to the 5′ or 3′ ends of the intron and can be present in small or very large introns, as opposed to box C/D snoRNAs, which are usually localized 60-90 nucleotides upstream the 3′-splice site and are encoded in relatively small introns. It has been suggested by Kiss and Filipowicz (1995, Genes Dev 9 (11): 141 1-1424) that a given snoRNA sequence could be excised and fully processed from an intronic region of any given actively spliced mRNA. To show the feasibility of this snoRNA processing independently from the host intron context, Kiss and Filipowicz artificially imbedded several snoRNAs (III 7a, U17b and U19) into the second intron of the human B-globin gene and expressed the resulting vector in fibroblast-like cells. After transfection, they found that the artificial, intronically delivered snoRNAs were properly processed from the human B-globin intron and the β-globin pre-mRNA was correctly spliced. Darzacq et al. (2002, EMBO J 21(11); 2746-2756) corroborated that other guide RNAs could be inserted into the second intron of the human β-globin gene using an expression vector under the control of the cytomegalovirus (CMV) promoter and be delivered to mammalian cells via transfection.

The inventors of the present application unexpectedly identified divergent host intron context-dependent effects on the pseudouridylation editing efficiency of different gsnoRNAs (as discussed in Example 1). For example, the present inventors tested the PTC readthrough efficiency of gsnoRNAs based on wildtype ACA19 (embedded in the host intron of EIF3A), ACA-44 (embedded in the host intron of SNHG12), ACA27 (embedded in the host intron of RPL21), and E2 (embedded in the host intron of RPSA) host genes, and embedded in a non-host intron of the HBB gene (FIGS. 2A and 2B). Surprisingly, the present inventors found that the editing efficiency of gsnoRNAs based on an E2 scaffold was lower when the gE2 was embedded in an HBB intron compared to the host RPSA intron, whereas the editing efficiency of gACA19 was similar when embedded in an HBB intron compared to the host EIF3A intron. Based on this observation that host gene sequences have divergent effects on different gsnoRNAs, the inventors envisioned that directly expressing the gsnoRNAs without host gene effects might further increase the efficiency of PTC-readthrough. Therefore, the inventors designed a series of gsnoRNA expression constructs wherein the nucleic acid molecule encoding the gsnoRNA is not embedded in an intron. As discussed in Example 1, the present inventors demonstrated enhanced pseudouridylation activity of gsnoRNAs not embedded in an intron, wherein the nucleic acid molecule encoding the gsnoRNA is driven by hU6 (type III RNA polymerase III promoter) and hU1 (snRNA-type RNA polymerase II promoter) promoters. Thus, in one aspect, provided herein is a nucleic acid molecule encoding a gsnoRNA, wherein the nucleic acid molecule is under the control of a small RNA promoter (e.g., a U6 or U1 promoter). In some embodiments, the nucleic acid encoding the gsnoRNA is not embedded in an intron sequence.

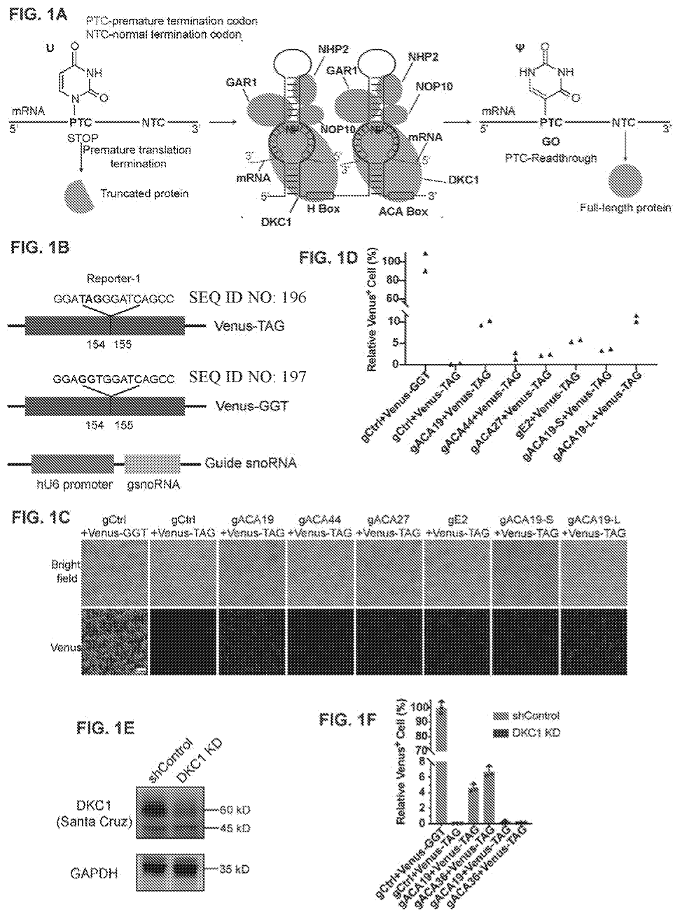

In some aspects, provided herein is a nucleic acid construct encoding the gsnoRNA. In some embodiments of the methods described herein, the method comprises introducing a nucleic acid molecule encoding the gsnoRNA into the host cell. In some embodiments, the nucleic acid molecule encoding the gsnoRNA is under the control of a small RNA promoter. In some embodiments, the small RNA promoter is a U6 (transcribed by Polymerase III) or U1 (transcribed by Polymerase II) promoter. In some embodiments, the expression of the gsnoRNA from the small RNA promoter according to the methods disclosed herein provides an increased pseudouridylation efficiency (e.g., an increased PTC-read-through efficiency) compared to the same gsnoRNA embedded in a host intron sequence or other intron sequence. In some embodiments, the pseudoridylation efficiency of the gsnoRNA expressed from a nucleic acid under the control of the small RNA promoter is 1.2-, 1.3-, 1.4-, 1.5-, 1.6-, 1.7-, 1.8-, 1.9- or 2-fold higher compared to the same gsnoRNA embedded in a host intron. Example 1 (FIGS. 1A-1E and FIGS. 2A-2C) provides results demonstrating enhanced PTC-read-through by a gsnoRNA expressed from a nucleic acid under the control of a small RNA promoter compared to an intron-embedded gsnoRNA.

In some embodiments, the nucleic acid molecule encoding the gsnoRNA is embedded in an intron sequence located between a first exon sequence and a second exon sequence. In some embodiments, the first exon sequence, the intron sequence and the second exon sequence are derived from a naturally-occurring gene. In some embodiments, the intron may comprise (besides the nucleic acid molecule of the present disclosure, comprising the guide region) additional nucleotides. Since the guide region is expressed from the intron sequence, such additional nucleotides may be selected to render the most efficient expression from the intron. In some embodiments, the exon A/intron/exon B sequence is present in a vector, such as a plasmid or a viral vector. Such a vector can be used to deliver the exon-intron-exon sequence to the cell. Additional introns and exons may be present in such a vector. In some embodiments, the exon A sequence (upstream of the intron that carries the nucleic acid encoding the gsnoRNA (which is expressed after transcription)) comprises or consists of exon 1 of the human β-globin gene, and the exon B sequence (downstream of the intron that carries the nucleic acid encoding the gsnoRNA (which is expressed after transcription)) comprises or consists of exon 2 of the human β-globin gene. In some embodiments, the exon A sequence (upstream of the intron that carries the nucleic acid encoding the gsnoRNA (which is expressed after transcription)) comprises or consists of exon 2 of the human Hemoglobin subunit β (HBB) gene, and the exon B sequence (downstream of the intron that carries the nucleic acid encoding the gsnoRNA (which is expressed after transcription)) comprises or consists of exon 3 of the human Hemoglobin subunit β (HBB) gene. In some embodiments, the nucleic acid molecule encoding the gsnoRNA is embedded in an intron sequence between a first exon sequence and a second exon sequence, wherein the intron sequence, first exon sequence, and second econ sequence correspond to the sequences of a naturally-occurring snoRNA-carrying host gene. In some embodiments, the construct comprising the intron-embedded gsnoRNA encoding sequence is under the control of a CMV promoter.

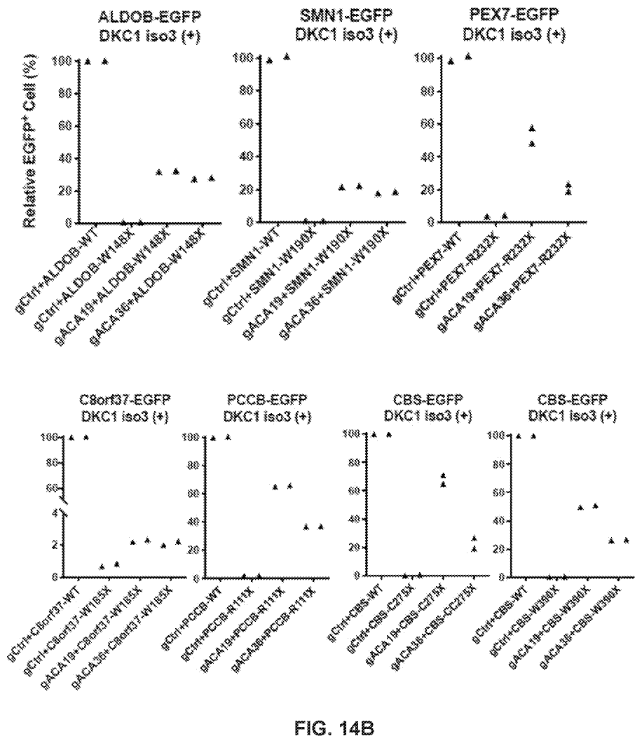

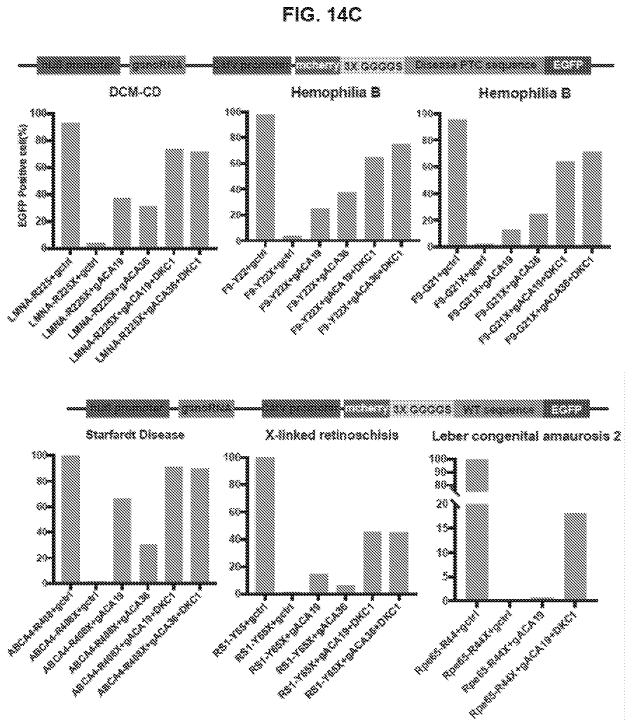

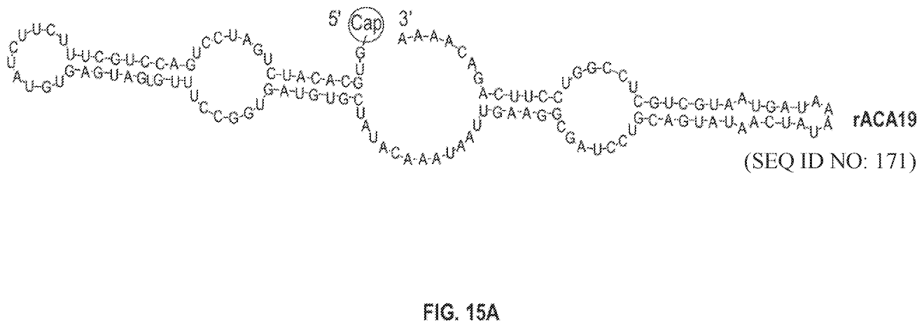

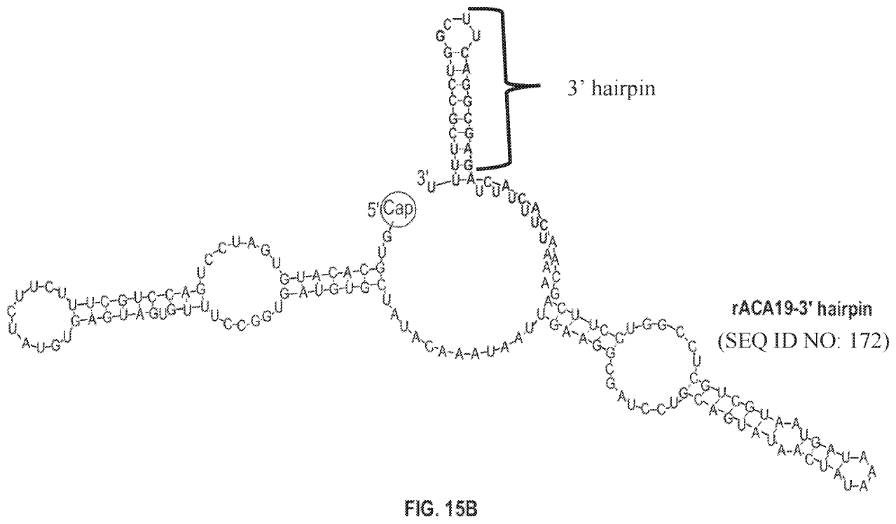

In some aspects, provided herein are engineered gsnoRNA targeting disease-associated PTCs. In some embodiments, the engineered gsnoRNA targeting disease-associated PTCs comprise one or more mutations to enhance the editing efficiency and/or expression of the gsnoRNAs. In some embodiments, the engineered gsnoRNA targeting disease-associated PTCs are selected from SEQ ID NOs: 71-84 (shown in FIGS. 14-15). Sequences of exemplary engineered gsnoRNAs targeting disease-associated PTCs are shown in Table 4 below.

In some embodiments, the gsnoRNA may be administered in a free form (or ‘naked’, without the context of a vector), or being delivered to a cell by other means, such as liposomes, or nanoparticles, or by using iontophoresis. In some embodiments, the gsnoRNA can be administered in a ribonucleoprotein complex (e.g., in a complex comprising DKC1, HNP2, NOP10, and/or GAR1). In some embodiments, the free gsnoRNA comprises one or more chemically modified nucleosides and/or inter-nucleosidic linkages as described above.

In some aspects, provided herein is a nucleic acid construct encoding DKC1 (e.g., any of the DKC1 proteins described in Section II A above). In some embodiments of the methods described herein, the method comprises introducing a nucleic acid molecule encoding the DKC1 protein into the host cell. In some embodiments, the nucleic acid molecule comprises a promoter operably linked to a nucleotide sequence encoding the DKC1. In some embodiments, the promoter is a Pol II promoter. In some embodiments, the promoter is a CMV promoter.

As disclosed herein, vectors may carry DNA or RNA, and are generally used to express the gsnoRNA and/or DKC1 protein constructs of the present disclosure after the vector is processed in the cell in which it is introduced. Such is generally through transcription of the DNA or RNA present in the vector. In some embodiments, vectors are viral vectors (that may be used to infect target cells to be treated), or plasmids, that may be introduced into the cell in a variety of ways, known to the person skilled in the art.

In some embodiments, the nucleic acid molecule encoding the DKC1 protein and/or the nucleic acid molecule encoding the gsnoRNA are present in a viral vector. In some embodiments, the method comprises introducing into the host cell a vector (e.g., a plasmid or viral vector) comprising a first nucleic acid sequence encoding the DKC1 protein and a second nucleic acid sequence encoding the gsnoRNA. In some embodiments, the vector is an adeno-associated viral (AAV) vector.

Exemplary engineered ACA scaffold sequences are shown in Table 2 below. The guide sequence is indicated as (Xn) and underlined, wherein Xn is a sequence of X nucleotides of length n, wherein X is any of A, U, G, or C and n is 4, 5, 6, 7, 8, 9, 10, 11, or 12. The guide sequence (Xn) can be modified to target the gsnoRNA to the desired target site, as will be understood by one of ordinary skill in the art. In some embodiments, n is an integer of a suitable length for the guide region. In some embodiments, n is 4, 5, 6, 7, 8, or 9.

Exemplary engineered gsnoRNA sequences, including exemplary guide sequences are shown in Table 3 below.

Exemplary engineered gsnoRNA sequences targeting exemplary disease-associated PTCs are shown in Table 4 below.

TABLE 2
ACA scaffold sequences.

| | | SEQ ID |
|---|---|---|
| Name | Sequence | NO. |
| gACA19 | GUGCACA(Xn)GACCUGCUUUCUUUUAUGUGAGUAGUGUU(Xn)GUG | 3 |
| | CUAUACAAAUAAUUGAAGGC(Xn)GCAGUAUAACUAUAAAUAGUAAU | |
| | GCUGC(Xn)CCUUCAGACAAAA | |
| gACA44 | CAGCA(Xn)GGGCUGUGGCUGGUCAUAGCCAUGGGAUC(Xn)GCAUG | 4 |
| | CAAGAGCAACCUGGAAAGA(Xn)ACAGCGCAGGUCAGUACAAUACCU | |
| | GCAAGCUGC(Xn)AGCUUUCCUAUAAUG | |
| gACA27 | UACCCC(Xn)GCCAGUUGGACUUAUGUCUUUAUUGGU(Xn)AGUGGG | 5 |
| | GCAAAGGAAAUAUCCUU(Xn)UCAGGCAAACUGGGUGUUUGUCUGUA | |
| | (Xn)GAGGAAACAAAU | |
| gE2 | UGUGCACA(Xn)GCUUGGAGUUGAGGCUACUGACUGGCCGAUGAACU | 6 |
| | CGCAAGU(Xn)GUGCUACAUGAGGGGCAAGU(Xn)ACACCACAAGGG | |
| | UCUCUGGCCCAAUGAGUGGAGUUUGA(Xn)AUUCUUGCUACAAGUA | |
| gACA19-S | CACA(Xn)GACCUGCUUUCUUUUAUGUGAGUAGUGUU(Xn)UGUGCU | 7 |
| | AUACAAAUAAUUGAAGGC(Xn)GCAGUAUAACUAUAAAUAGUAAUGC | |
| | UGC(Xn)CCUUCAGACAAAA | |
| gACA19-L | UCAGUAUUUGUGCACA(Xn)GACCUGCUUUCUUUUAUGUGAGUAGUG | 8 |
| | UU(Xn)GUGCUAUACAAAUAAUUGAAGGC(Xn)GCAGUAUAACUAUA | |
| | AAUAGUAAUGCUGC(Xn)CCUUCAGACAAAAAUUCUAUAA | |
| gACA3 | AUCGA(Xn)ACGCUUGGGUAUCGGCUAUUGCCUGAGUGU(Xn)CCUC | 9 |
| | GAAGAGUAACUGCUGAC(Xn)ACUGGCUGUGGGCCUUAUGGCACAGU | |
| | CAGU(Xn)CAGGUUAGAGACAUGC | |
| gACA17 | ACUGCCCCU(Xn)GCAGCUGUGGCUGCCGUGUCACAUCUGU(Xn)GU | 10 |
| | GGCAGAGAUUAGAGAGGCUAUGU(Xn)CAAGCGUUCUGCCCCGUGAA | |
| | CGUUUG(Xn)GUCUCACACUC | |
| gACA2b | UUGGCUCU(Xn)GGCCAGCAGUUUGCUGAAGCUGUUGGcc(Xn)CAG | 11 |
| | GAGCCUAAAGAAUUGUCUUUCUA(Xn)UUGGCCAUUUCAUAACUUUG | |
| | GAAAUGUAAUGGUCAA(Xn)AGAAAGAAACAUGA | |
| gACA36 | UUCCAAA(Xn)UCAGUCCAGGGCAGCUUCCCUGUUCUGA(Xn)UUUG | 12 |
| | GGACAUUAAAAUGGGCUAAGGGAG(Xn)GGGUAGAAAGUAUUAUUCU | |
| | AUUC(Xn)CCUCCCAGCCUACAAAA | |
| gACA19- | GUGCACA(Xn)GACCUGCUUUCUUCUAUGUGAGUAGUGUU(Xn)GUG | 15 |
| UUCU | CUAUACAAAUAAUUGAAGGC(Xn)GCAGUAUAACUAUAAAUAGUAAU | |
| | GCUGC(Xn)CCUUCAGACAAAA | |
| gACA19- | GUGCACA(Xn)GACCUGCUUUCUGUUAUGUGAGUAGUGUU(Xn)GUG | 16 |
| UGUU | CUAUACAAAUAAUUGAAGGC(Xn)GCAGUAUAACUAUAAAUAGUAAU | |
| | GCUGC(Xn)CCUUCAGACAAAA | |
| gACA19- | GUGCACA(Xn)GACCUGCUUUUUUUAUGUGAGUAGUGUU(Xn)GUG | 17 |
| 3addG | CUAUACAAAUAAUUGAAGGC(Xn)GCAGUAUAACUAUAAAUAGUAAU | |
| | GCUGC(Xn)GCCUUCAGACAAAA | |
| gACA19- | GUGCACA(Xn)GACCUGCUUUCUUUUAUGUGAGUAGUGUU(Xn)GUG | 18 |
| 3addUG | CUAUACAAAUAAUUGAAGGCU(Xn)GCAGUAUAACUAUAAAUAGUAA | |
| | UGCUGC(Xn)CCUUCAGACAAAA | |
| gACA19- | GUGCACAUCU(Xn)GACCUGCUUUCUUUUAUGUGAGUAGUGUU(Xn) | 19 |
| 5addCU | GUGCUAUACAAAUAAUUGAAGGC(Xn)GCAGUAUAACUAUAAAUAGU | |
| | AAUGCUGC(Xn)CCUUCAGACAAAA | |
| gACA36- | UUCCAAA(Xn)UCAGUCCAGGGCAGCUUCCCUGUACUGA(Xn)UUUG | 22 |
| 5UmA | GGACAUUAAAAUGGGCUAAGGGAG(Xn)GGGUAGAAAGUAUUAUUCU | |
| | AUUC(Xn)CCUCCCAGCCUACAAAA | |
| gACA36- | UUCCAAA(Xn)UCAGUCCAGGGCAGCUUCCCUGGACUGA(Xn)UUUG | 23 |
| 5UUmGA | GGACAUUAAAAUGGGCUAAGGGAG(Xn)GGGUAGAAAGUAUUAUUCU | |
| | AUUC(Xn)CCUCCCAGCCUACAAAA | |
| gACA36- | UUCCAAG(Xn)UCAGUCCAGGGCAGCUUCCCUGUUCUGA(Xn)CUUG | 24 |
| 5CGbp | GGACAUUAAAAUGGGCUAAGGGAG(Xn)GGGUAGAAAGUAUUAUUCU | |
| | AUUC(Xn)CCUCCCAGCCUACAAAA | |
| gACA36- | UUCCAAA(Xn)UCAGUCCAGGGCAGCUUCCCUGUUCUGA(Xn)UUUG | 25 |
| 3GmU | GGACAUUAAAAUGGGCUAAGGGA(Xn)GGGUAGAAAGUAUUAUUCUA | |
| | UUC(Xn)CCUCCCAGCCUACAAAA | |
| gACA36- | UUCCAAA(Xn)UCAGUCCAGGGCAGCUUCCCUGUUCUGA(Xn)UUUG | 26 |
| 3GmU- | GGACAUUAAAAUGGGCUGGGA(Xn)GGGUAGAAAGUAUUAUUCUAUU | |
| delAA | C(Xn)CCUCCCAGCCUACAAAA | |
| gACA36- | UUCCAAA(Xn)UCAGUCCAGGGCAGCUUCCCUGUUCUGA(Xn)UUUG | 27 |
| 3GmU- | GGACAUUAAAAUGGGCUAGGGA(Xn)GGGUAGAAAGUAUUAUUCUAU | |
| delA | UC(Xn)CCUCCCAGCCUACAAAA | |
| gACA36- | UUCCAAA(Xn)UCAGUCCAGGGCAGCUUCCCUGUUCUGA(Xn)UUUG | 28 |
| 3UmC | GGACAUUAAAAUGGGCUAAGGGAG(Xn)GGGUAGAAAGUAUUAUUCU | |
| | AUCC(Xn)CCUCCCAGCCUACAAAA | |
| gACA36- | UUCCAAA(Xn)UCAGUCCAGGGCAGCUUCCCUGUUCUGA(Xn)UUUG | 29 |
| 3GmU- | GGACAUUAAAAUGGGCUGGGA(Xn)GGGUAGAAAGUAUUAUUCUAUC | |
| delAA- | C(Xn)CCUCCCAGCCUACAAAA | |
| UmC | | |
| gACA36- | UUCCAAA(Xn)UCAGUCCAGGGCAGCUUCCCUGUUCUGA(Xn)UUUG | 30 |
| 3delGC | GGACAUUAAAAUGGCUAAGGGAG(Xn)GGGUAGAAAGUAUUAUUCUA | |
| | UUC(Xn)CCUCCCAGCUACAAAA | |
| gACA36- | UUCCAAA(Xn)UCAGUCCAGGGCAGCUUCCCUGUUCUGA(Xn)UUUG | 31 |
| 3delC | GGACAUUAAAAUGGGCUAAGGGAG(Xn)GGGUAGAAAGUAUUAUUCU | |
| | AUUC(Xn)CUCCCAGCCUACAAAA | |
| gACA36- | UUCCAAA(Xn)UCAGUCCAGGGCAGCUUCCCUGUUCUGA(Xn)UUUG | 32 |
| 3GmU- | GGACAUUAAAAUGGGCUAAGGGA(Xn)GGGUAGAAAGUAUUAUUCUA | |
| delC | UUC(Xn)CUCCCAGCCUACAAAA | |
| gACA19- | GUGCACA(Xn)GACCUGCUUUCUUCUAUGUGAGUAGUGUU(Xn)UGU | 33 |
| UUCU- | GCUAUACAAAUAAUUGAAGGC(Xn)GCAGUAUAACUAUAAAUAGUAA | |
| 3addG | UGCUGC(Xn)CCUUCAGACAAAA | |
| gACA19- | GUGCACAU(Xn)GACCUGCUUUCUUCUAUGUGAGUAGUGUU(Xn)GU | 34 |
| UUCU- | GCUAUACAAAUAAUUGAAGGC(Xn)GCAGUAUAACUAUAAAUAGUAA | |
| SaddCU | UGCUGC(Xn)CCUUCAGACAAAA | |
| gACA19- | GUGCACAU(Xn)GACCUGCUUUCUUUUAUGUGAGUAGUGUU(Xn)GU | 35 |
| 3addG- | GCUAUACAAAUAAUUGAAGGC(Xn)GCAGUAUAACUAUAAAUAGUAA | |
| SaddCU | UGCUGC(Xn)CCUUCAGACAAAA | |
| gACA19- | GUGCACAUC(Xn)GACCUGCUUUCUUCUAUGUGAGUAGUGUU(Xn)G | 36 |
| UUCU- | UGCUAUACAAAUAAUUGAAGGC( Xn)GCAGUAUAACUAUAAAUAGUA | |
| 3addG- | AUGCUGC(Xn)CCUUCAGACAAAA | |
| SaddCU | | |
| gACA2b- | UUGGCUCUC(Xn)GGCCAGCAGUUUGCUGAAGCUGUUGGcc(Xn)CA | 20 |
| SaddC | GGAGCCUAAAGAAUUGUCUUUCUA(Xn)UUGGCCAUUUCAUAACUUU | |
| | GGAAAUGUAAUGGUCAA(Xn)AGAAAGAAACAUGA | |
| gACA2b- | UUGGCU(Xn)GGCCAGCAGUUUGCUGAAGCUGUUGGcc(Xn)CAGGA | 21 |
| 5CUmGC | GCCUAAAGAAUUGUCUUUCUA(Xn)UUGGCCAUUUCAUAACUUUGGA | |
| | AAUGUAAUGGUCAA(Xn)AGAAAGAAACAUGA | |
| gACA2b- | UUGGCUCU(Xn)GGCCAGCAGUUUGCUGAAGCUGUUGGcc(Xn)GGA | 145 |
| SCAmUG | GCCUAAAGAAUUGUCUUUCUA(Xn)UUGGCCAUUUCAUAACUUUGGA | |
| | AAUGUAAUGGUCAA(Xn)AGAAAGAAACAUGA | |
| gACA2b- | UUGGCUCUC(Xn)GGCCAGCAGUUUGCUGAAGCUGUUGGcc(Xn)GG | 146 |
| SaddC- | AGCCUAAAGAAUUGUCUUUCUA(Xn)UUGGCCAUUUCAUAACUUUGG | |
| 5CAmUG | AAAUGUAAUGGUCAA(Xn)AGAAAGAAACAUGA | |
| gACA2b- | UUGGCU(Xn)GGCCAGCAGUUUGCUGAAGCUGUUGGcc(Xn)GGAGC | 147 |
| 5CUmGC- | CUAAAGAAUUGUCUUUCUA(Xn)UUGGCCAUUUCAUAACUUUGGAAA | |
| 5CAmUG | UGUAAUGGUCAA(Xn)AGAAAGAAACAUGA | |
| gACA2b- | UUGGCUCU(Xn)GGCCAGCAGUUUGCUGAAGCUGUUGGcc(Xn)CAG | 148 |
| 3delA | GAGCCUAAAGAAUUGUCUUUCU(Xn)UUGGCCAUUUCAUAACUUUGG | |
| | AAAUGUAAUGGUCAA(Xn)AGAAAGAAACAUGA | |
| gACA2b- | UUGGCUCU(Xn)GGCCAGCAGUUUGCUGAAGCUGUUGGcc(Xn)CAG | 149 |
| 3delA- | GAGCCUAAAGAAUUGUCGUUCU(Xn)UUGGCCAUUUCAUAACUUUGG | |
| GCbp-2 | AAAUGUAAUGGUCAA(Xn)AGAACGAAACAUGA | |
| gACA2b- | UUGGCUCU(Xn)GGCCAGCAGUUUGCUGAAGCUGUUGGcc(Xn)CAG | 150 |
| GCbp | GAGCCUAAAGAAUUGUCUUUCUA(Xn)GUGGCCAUUUCAUAACUUUG | |
| | GAAAUGUAAUGGUCAC(Xn)AGAAAGAAACAUGA | |
| rACA19- | GUGCACAU(Xn)GACCUGCUUUCUUCUAUGUGAGUAGUGUU(Xn)GU | 177 |
| 3'hairpin | GCUAUACAAAUAAUUGAAGGC(Xn)GCAGUAUAACUAUAAAUAGUAA | |
| | UGCUGC(Xn)CCUUCAGACAAAAUCUAUCUAUCUAGAGCGGACUUCG | |
| | GUCCGCUUUU | |
| rACA19- | GUGCUCGCUUCGGCAGCACAUAUACUAGUGCACAU(Xn)GACCUGCU | 178 |
| U6+27 | UUCUUCUAUGUGAGUAGUGUU(Xn)GUGCUAUACAAAUAAUUGAAGG | |
| | C(Xn)GCAGUAUAACUAUAAAUAGUAAUGCUGC(Xn)CCUUCAGACA | |
| | AAAUCUAGAGCGGACUUCGGUCCGCUUUU | |
| rH5 | GUGCACAU(Xn)GACCUGCUUUCUUCUAUGUGAGUAGUGUU(Xn)GU | 179 |
| | GCUAUACAA | |
| rH3 | GGAAUUGAAGGC(Xn)GCAGUAUAACUAUAAAUAGUAAUGCUGC | 180 |
| | (Xn)CCUUCAGACAAAA | |

TABLE 3
Exemplary gsnoRNA constructs(guide sequences are underlined)

| | | SEQ ID |
|---|---|---|
| Name | Sequence | NO. |
| gACA19 | GUGCACAUGAUCCCGACCUGCUUUCUUUUAUGUGAGUAGUGUUUCCG | 37 |
| | GUGAUGUGCUAUACAAAUAAUUGAAGGCGAUCCCGCAGUAUAACUAU | |
| | AAAUAGUAAUGCUGCUCCGGUCCUUCAGACAAAA | |
| gACA44 | CAGCAGCUGAUCCCGGGCUGUGGCUGGUCAUAGCCAUGGGAUCUCCG | 38 |
| | GUGGCAUGCAAGAGCAACCUGGAAAGAAUCCCACAGCGCAGGUCAGU | |
| | ACAAUACCUGCAAGCUGCUCCGGAGCUUUCCUAUAAUG | |
| gACA27 | UACCCCCGGCUGAUCCCGCCAGUUGGACUUAUGUCUUUAUUGGUUCC | 39 |
| | GGUAGUGGGGCAAAGGAAAUAUCCUUUGAUCCCUCAGGCAAACUGGG | |
| | UGUUUGUCUGUAUCCGGUGAGAGGAAACAAAU | |
| gE2 | UGUGCACACUGAUCCCGCUUGGAGUUGAGGCUACUGACUGGCCGAUG | 40 |
| | AACUCGCAAGUUCCGGUGAUGUGCUACAUGAGGGGCAAGUCUGAUCC | |
| | CACACCACAAGGGUCUCUGGCCCAAUGAGUGGAGUUUGAUCCGGAUU | |
| | CUUGCUACAAGUA | |
| gACA19-S | CACAUGAUCCCGACCUGCUUUCUUUUAUGUGAGUAGUGUUUCCGGUG | 41 |
| | AUGUGCUAUACAAAUAAUUGAAGGCGAUCCCGCAGUAUAACUAUAAA | |
| | UAGUAAUGCUGCUCCGGUCCUUCAGACAAAA | |
| gACA19-L | UCAGUAUUUGUGCACAUGAUCCCGACCUGCUUUUUUUAUGUGAGUA | 42 |
| | GUGUUUCCGGUGAUGUGCUAUACAAAUAAUUGAAGGCGAUCCCGCAG | |
| | UAUAACUAUAAAUAGUAAUGCUGCUCCGGUCCUUCAGACAAAAAUUC | |
| | UAUAA | |
| gACA3 | AUCGAGGCUGAUCCCACGCUUGGGUAUCGGCUAUUGCCUGAGUGUUC | 43 |
| | CGGUGACCUCGAAGAGUAACUGCUGACUGAUCCCACUGGCUGUGGGC | |
| | CUUAUGGCACAGUCAGUUCCGCAGGUUAGAGACAUGC | |
| gACA17 | ACUGCCCCUCUGAUCCCGCAGCUGUGGCUGCCGUGUCACAUCUGUUC | 44 |
| | CGGUGAGUGGCAGAGAUUAGAGAGGCUAUGUUGAUCCCCAAGCGUUC | |
| | UGCCCCGUGAACGUUUGUCCGGUGAUAGUCUCACACUC | |
| gACA2b | UUGGCUCUUGAUCCCGGCCAGCAGUUUGCUGAAGCUGUUGGccUCCG | 45 |
| | GCAGGAGCCUAAAGAAUUGUCUUUCUAUGAUCCCUUGGCCAUUUCAU | |
| | AACUUUGGAAAUGUAAUGGUCAAUCCGGUAGAAAGAAACAUGA | |
| gACA36 | UUCCAAAGCUGAUCCCUCAGUCCAGGGCAGCUUCCCUGUUCUGAUCC | 46 |
| | GGUGAUUUGGGACAUUAAAAUGGGCUAAGGGAGGAUCCCGGGUAGAA | |
| | AGUAUUAUUCUAUUCUCCGCCUCCCAGCCUACAAAA | |
| gACA19- | GUGCACAUGUGCUAGACCUGCUUUCUUUUAUGUGAGUAGUGUUGCUG | 47 |
| 5m | UUAAUGUGCUAUACAAAUAAUUGAAGGCGAUCCCGCAGUAUAACUAU | |
| | AAAUAGUAAUGCUGCUCCGGUCCUUCAGACAAAA | |
| gACA19- | GUGCACAUGAUCCCGACCUGCUUUCUUUUAUGUGAGUAGUGUUUCCG | 48 |
| 3m | GUGAUGUGCUAUACAAAUAAUUGAAGGCGACCGGGCAGUAUAACUAU | |
| | AAAUAGUAAUGCUGCAGCUAUCCUUCAGACAAAA | |
| gACA19- | GUGCACAUGAUCCCGACCUGCUUUCUUCUAUGUGAGUAGUGUUUCCG | 49 |
| UUCU | GUGAUGUGCUAUACAAAUAAUUGAAGGCGAUCCCGCAGUAUAACUAU | |
| | AAAUAGUAAUGCUGCUCCGGUCCUUCAGACAAAA | |
| gACA19- | GUGCACAUGAUCCCGACCUGCUUUCUGUUAUGUGAGUAGUGUUUCCG | 50 |
| UGUU | GUGAUGUGCUAUACAAAUAAUUGAAGGCGAUCCCGCAGUAUAACUAU | |
| | AAAUAGUAAUGCUGCUCCGGUCCUUCAGACAAAA | |
| gACA19- | GUGCACAUGAUCCCGACCUGCUUUCUUUUAUGUGAGUAGUGUUUCCG | 51 |
| 3addG | GUGAUGUGCUAUACAAAUAAUUGAAGGCGAUCCCGCAGUAUAACUAU | |
| | AAAUAGUAAUGCUGCUCCGGUGCCUUCAGACAAAA | |
| gACA19- | GUGCACAUGAUCCCGACCUGCUUUCUUUUAUGUGAGUAGUGUUUCCG | 52 |
| 3addUG | GUGAUGUGCUAUACAAAUAAUUGAAGGCUGAUCCCGCAGUAUAACUA | |
| | UAAAUAGUAAUGCUGCUCCGGUGCCUUCAGACAAAA | |
| gACA19- | GUGCACAUCUGAUCCCGACCUGCUUUCUUUUAUGUGAGUAGUGUUUC | 53 |
| 5addCU | CGGUGAUGUGCUAUACAAAUAAUUGAAGGCGAUCCCGCAGUAUAACU | |
| | AUAAAUAGUAAUGCUGCUCCGGUCCUUCAGACAAAA | |
| gACA36- | UUCCAAAGCUCUAAGAUCAGUCCAGGGCAGCUUCCCUGUUCUGAGUA | 54 |
| 5m | AUUGAUUUGGGACAUUAAAAUGGGCUAAGGGAGGAUCCCGGGUAGAA | |
| | AGUAUUAUUCUAUUCUCCGCCUCCCAGCCUACAAAA | |
| gACA36- | UUCCAAAGCUGAUCCCUCAGUCCAGGGCAGCUUCCCUGUUCUGAUCC | 55 |
| 3m | GGUGAUUUGGGACAUUAAAAUGGGCUAAGGGAGGUAAGAGGGUAGAA | |
| | AGUAUUAUUCUAUUCGUAUCCUCCCAGCCUACAAAA | |
| gACA36- | UUCCAAAGCUGAUCCCUCAGUCCAGGGCAGCUUCCCUGUACUGAUCC | 56 |
| 5UmA | GGUGAUUUGGGACAUUAAAAUGGGCUAAGGGAGGAUCCCGGGUAGAA | |
| | AGUAUUAUUCUAUUCUCCGCCUCCCAGCCUACAAAA | |
| gACA36- | UUCCAAAGCUGAUCCCUCAGUCCAGGGCAGCUUCCCUGGACUGAUCC | 57 |
| SUUmGA | GGUGAUUUGGGACAUUAAAAUGGGCUAAGGGAGGAUCCCGGGUAGAA | |
| | AGUAUUAUUCUAUUCUCCGCCUCCCAGCCUACAAAA | |
| gACA36- | UUCCAAGGCUGAUCCCUCAGUCCAGGGCAGCUUCCCUGUUCUGAUCC | 58 |
| 5CGbp | GGUGACUUGGGACAUUAAAAUGGGCUAAGGGAGGAUCCCGGGUAGAA | |
| | AGUAUUAUUCUAUUCUCCGCCUCCCAGCCUACAAAA | |
| gACA36- | UUCCAAAGCUGAUCCCUCAGUCCAGGGCAGCUUCCCUGUUCUGAUCC | 59 |
| 3GmU | GGUGAUUUGGGACAUUAAAAUGGGCUAAGGGAUGAUCCCGGGUAGAA | |
| | AGUAUUAUUCUAUUCUCCGCCUCCCAGCCUACAAAA | |
| gACA36- | UUCCAAAGCUGAUCCCUCAGUCCAGGGCAGCUUCCCUGUUCUGAUCC | 60 |
| 3GmU- | GGUGAUUUGGGACAUUAAAAUGGGCUGGGAUGAUCCCGGGUAGAAAG | |
| delAA | UAUUAUUCUAUUCUCCGCCUCCCAGCCUACAAAA | |
| gACA36- | UUCCAAAGCUGAUCCCUCAGUCCAGGGCAGCUUCCCUGUUCUGAUCC | 61 |
| 3GmU- | GGUGAUUUGGGACAUUAAAAUGGGCUAGGGAUGAUCCCGGGUAGAAA | |
| delA | GUAUUAUUCUAUUCUCCGCCUCCCAGCCUACAAAA | |
| gACA36- | UUCCAAAGCUGAUCCCUCAGUCCAGGGCAGCUUCCCUGUUCUGAUCC | 62 |
| 3UmC | GGUGAUUUGGGACAUUAAAAUGGGCUAAGGGAGGAUCCCGGGUAGAA | |
| | AGUAUUAUUCUAUCCUCCGCCUCCCAGCCUACAAAA | |
| gACA36- | UUCCAAAGCUGAUCCCUCAGUCCAGGGCAGCUUCCCUGUUCUGAUCC | 63 |
| 3GmU- | GGUGAUUUGGGACAUUAAAAUGGGCUGGGAUGAUCCCGGGUAGAAAG | |
| delAA- | UAUUAUUCUAUCCUCCGCCUCCCAGCCUACAAAA | |
| UmC | | |
| gACA36- | UUCCAAAGCUGAUCCCUCAGUCCAGGGCAGCUUCCCUGUUCUGAUCC | 64 |
| 3delGC | GGUGAUUUGGGACAUUAAAAUGGCUAAGGGAGGAUCCCGGGUAGAAA | |
| | GUAUUAUUCUAUUCUCCGCCUCCCAGCUACAAAA | |
| gACA36- | UUCCAAAGCUGAUCCCUCAGUCCAGGGCAGCUUCCCUGUUCUGAUCC | 65 |
| 3delC | GGUGAUUUGGGACAUUAAAAUGGGCUAAGGGAGGAUCCCGGGUAGAA | |
| | AGUAUUAUUCUAUUCUCCGCUCCCAGCCUACAAAA | |
| gACA36- | UUCCAAAGCUGAUCCCUCAGUCCAGGGCAGCUUCCCUGUUCUGAUCC | 66 |
| 3GmU- | GGUGAUUUGGGACAUUAAAAUGGGCUAAGGGAUGAUCCCGGGUAGAA | |
| delC | AGUAUUAUUCUAUUCUCCGCUCCCAGCCUACAAAA | |
| gACA19- | GUGCACAUGAUCCCGACCUGCUUUCUUCUAUGUGAGUAGUGUUUCCG | 67 |
| UUCU- | GUGAUGUGCUAUACAAAUAAUUGAAGGCGAUCCCGCAGUAUAACUAU | |
| 3addG | AAAUAGUAAUGCUGCUCCGGUGCCUUCAGACAAAA | |
| gACA19- | GUGCACAUCUGAUCCCGACCUGCUUUCUUCUAUGUGAGUAGUGUUUC | 68 |
| UUCU- | CGGUGAUGUGCUAUACAAAUAAUUGAAGGCGAUCCCGCAGUAUAACU | |
| 5addCU | AUAAAUAGUAAUGCUGCUCCGGUCCUUCAGACAAAA | |
| gACA19- | GUGCACAUCUGAUCCCGACCUGCUUUCUUUUAUGUGAGUAGUGUUUC | 69 |
| 3addG- | CGGUGAUGUGCUAUACAAAUAAUUGAAGGCGAUCCCGCAGUAUAACU | |
| SaddCU | AUAAAUAGUAAUGCUGCUCCGGUGCCUUCAGACAAAA | |
| gACA19- | GUGCACAUCUGAUCCCGACCUGCUUUCUUCUAUGUGAGUAGUGUUUC | 70 |
| UUCU- | CGGUGAUGUGCUAUACAAAUAAUUGAAGGCGAUCCCGCAGUAUAACU | |
| 3addG- | AUAAAUAGUAAUGCUGCUCCGGUGCCUUCAGACAAAA | |
| 5addCU | | |
| gACA2b- | UUGGCUCUUGUAAGAGGCCAGCAGUUUGCUGAAGCUGUUGGccGUAC | 151 |
| 5m | UCAGGAGCCUAAAGAAUUGUCUUUCUAUGAUCCCUUGGCCAUUUCAU | |
| | AACUUUGGAAAUGUAAUGGUCAAUCCGGUAGAAAGAAACAUGA | |
| gACA2b- | UUGGCUCUUGAUCCCGGCCAGCAGUUUGCUGAAGCUGUUGGCCUCCG | 152 |
| 3m | GCAGGAGCCUAAAGAAUUGUCUUUCUAUGUAGGAUUGGCCAUUUCAU | |
| | AACUUUGGAAAUGUAAUGGUCAAGUGAGUAGAAAGAAACAUGA | |
| gACA2b- | UUGGCUCUCUGAUCCCGGCCAGCAGUUUGCUGAAGCUGUUGGccUCC | 153 |
| SaddC | GGCAGGAGCCUAAAGAAUUGUCUUUCUAUGAUCCCUUGGCCAUUUCA | |
| | UAACUUUGGAAAUGUAAUGGUCAAUCCGGUAGAAAGAAACAUGA | |
| gACA2b- | UUGGCUGCUGAUCCCGGCCAGCAGUUUGCUGAAGCUGUUGGccUCCG | 154 |
| 5CUmGC | GCAGGAGCCUAAAGAAUUGUCUUUCUAUGAUCCCUUGGCCAUUUCAU | |
| | AACUUUGGAAAUGUAAUGGUCAAUCCGGUAGAAAGAAACAUGA | |
| gACA2b- | UUGGCUCUUGAUCCCGGCCAGCAGUUUGCUGAAGCUGUUGGccUCCG | 156 |
| 5CAmUG | GUGGGAGCCUAAAGAAUUGUCUUUCUAUGAUCCCUUGGCCAUUUCAU | |
| | AACUUUGGAAAUGUAAUGGUCAAUCCGGUAGAAAGAAACAUGA | |
| gACA2b- | UUGGCUCUCUGAUCCCGGCCAGCAGUUUGCUGAAGCUGUUGGccUCC | 157 |
| SaddC- | GGUGGGAGCCUAAAGAAUUGUCUUUCUAUGAUCCCUUGGCCAUUUCA | |
| 5CAmUG | UAACUUUGGAAAUGUAAUGGUCAAUCCGGUAGAAAGAAACAUGA | |
| gACA2b- | UUGGCUGCUGAUCCCGGCCAGCAGUUUGCUGAAGCUGUUGGccUCCG | 158 |
| 5CUmGC- | GUGGGAGCCUAAAGAAUUGUCUUUCUAUGAUCCCUUGGCCAUUUCAU | |
| 5CAmUG | AACUUUGGAAAUGUAAUGGUCAAUCCGGUAGAAAGAAACAUGA | |
| gACA2b- | UUGGCUCUUGAUCCCGGCCAGCAGUUUGCUGAAGCUGUUGGccUCCG | 159 |
| 3delA | GCAGGAGCCUAAAGAAUUGUCUUUCUUGAUCCCUUGGCCAUUUCAUA | |
| | ACUUUGGAAAUGUAAUGGUCAAUCCGGUAGAAAGAAACAUGA | |
| gACA2b- | UUGGCUCUUGAUCCCGGCCAGCAGUUUGCUGAAGCUGUUGGccUCCG | 160 |
| 3delA- | GCAGGAGCCUAAAGAAUUGUCGUUCUUGAUCCCUUGGCCAUUUCAUA | |
| GCbp-2 | ACUUUGGAAAUGUAAUGGUCAAUCCGGUAGAACGAAACAUGA | |
| gACA2b- | UUGGCUCUUGAUCCCGGCCAGCAGUUUGCUGAAGCUGUUGGccUCCG | 170 |
| GCbp | GCAGGAGCCUAAAGAAUUGUCUUUCUAUGAUCCCGUGGCCAUUUCAU | |
| | AACUUUGGAAAUGUAAUGGUCACUCCGGUAGAAAGAAACAUGA | |
| rACA19 | GUGCACAUCUGAUCCUGACCUGCUUUCUUCUAUGUGAGUAGUGUUUC | 171 |
| | CGGUGAUGUGCUAUACAAAUAAUUGAAGGCGAUCCUGCAGUAUAACU | |
| | AUAAAUAGUAAUGCUGCUCCGGUCCUUCAGACAAAA | |
| rACA19- | GUGCACAUCUGAUCCUGACCUGCUUUCUUCUAUGUGAGUAGUGUUUC | 172 |
| 3′hairpin | CGGUGAUGUGCUAUACAAAUAAUUGAAGGCGAUCCUGCAGUAUAACU | |
| | AUAAAUAGUAAUGCUGCUCCGGUCCUUCAGACAAAAUCUAUCUAUCU | |
| | AGAGCGGACUUCGGUCCGCUUUU | |
| rACA19- | GUGCUCGCUUCGGCAGCACAUAUACUAGUGCACAUCUGAUCCUGACC | 173 |
| U6+27 | UGCUUUCUUCUAUGUGAGUAGUGUUUCCGGUGAUGUGCUAUACAAAU | |
| | AAUUGAAGGCGAUCCUGCAGUAUAACUAUAAAUAGUAAUGCUGCUCC | |
| | GGUCCUUCAGACAAAAUCUAGAGCGGACUUCGGUCCGCUUUU | |
| rH5 | GUGCACAUCUGAUCCUGACCUGCUUUCUUCUAUGUGAGUAGUGUUUC | 174 |
| | CGGUGAUGUGCUAUACAA | |
| rH3 | GGAAUUGAAGGCGAUCCUGCAGUAUAACUAUAAAUAGUAAUGCUGCU | 175 |
| | CCGGUCCUUCAGACAAAA | |

TABLE 4
Disease-associated PTC targeting gsnoRNA
(guide sequences are underlined)

| | | SEQ ID |
|---|---|---|
| Name | Sequence | NO. |
| gACA19- | GUGCACAUcGgcacgUGACCUGCUUUCUUCUAUGUGAGUAGUGUUcU | 71 |
| ALDOB- | UcccaAUGUGCUAUACAAAUAAUUGAAGGCgcacgUGCAGUAUAACU | |
| W148X | AUAAAUAGUAAUGCUGCcUUccUCCUUCAGACAAAA | |
| gACA36- | UUCCAAAGcGgcacgUUCAGUCCAGGGCAGCUUCCCUGUUCUGAUUU | 72 |
| ALDOB- | ccUaaUUUGGGACAUUAAAAUCGGCUGGUGagcacgUGGACUAAGAA | |
| W148X | AGUAUUAUUCAUAGUCCcUUcccGACCAGCCUACAAAA | |
| gACA19- | GUGCACAUaagagUUUGACCUGCUUUCUUCUAUGUGAGUAGUGUUUg | 73 |
| SMN1- | gagUaAUGUGCUAUACAAAUAAUUGAAGGCgagUUUGCAGUAUAACU | |
| W190X | AUAAAUAGUAAUGCUGCUggagUCCUUCAGACAAAA | |
| gACA36- | UUCCAAAGaagagUUUACAGUCCAGGGCAGCUUCCCUGUUCUGUUgg | 74 |
| SMN1- | agUagUUUGGGACAUUAAAAUCGGCUGGUGagagUUUGGACUAAGAA | |
| W190X | AGUAUUAUUCAUAGUCCUggagcGACCAGCCUACAAAA | |
| gACA19- | GUGCACAUUGgUUcUUGACCUGCUUUCUUCUAUGUGAGUAGUGUUgc | 75 |
| C8orf37- | UaUacAUGUGCUAUACAAAUAAUUGAAGGagUUcUUGCAGUAUAACU | |
| W185X | AUAAAUAGUAAUGCUGCgcUaUaCCUUCAGACAAAA | |
| gACA36- | UUCCAAACUagUUcUUUCAGUCCAGGGCAGCUUCCCUGUUCUGAgcU | 76 |
| C8orf37- | acacAUUUGGGACAUUAAAAUGGGCUAAGGGUagUUcUUGGGUAGAA | |
| W185X | AGUAUUAUUCUAUUCgcUaUaUCCCAGCCUACAAAA | |
| gACA19- | GUGCACAUCgaUUcUUGACCUGCUUUCUUCUAUGUGAGUAGUGUUUc | 77 |
| CBS- | cgggGAUGUGCUAUACAAAUAAUUGAAGGgaUUcUUGCAGUAUAACU | |
| C275X | AUAAAUAGUAAUGCUGCUccgggCCUUCAGACAAAA | |
| gACA36- | UUCCAAAUUgaUcUUUUCAGUCCAGGGCAGCUUCCCUGUUCUGAUcc | 78 |
| CBS- | gggacUUUGGGACAUUAAAAUCGGCUGGUGgaUUcUUGGACUAAGAA | |
| C275X | AGUAUUAUUCAUAGUCCUccgggaACCAGCCUACAAAA | |
| gACA19- | GUGCACAUgUagUgUUGACCUGCUUUCUUCUAUGUGAGUAGUGUUcc | 79 |
| CBS- | UgUcCAUGUGCUAUACAAAUAAUUGAAGGUagUgUUGCAGUAUAACU | |
| W390X | AUAAAUAGUAAUGCUGCccUgUcCCUUCAGACAAAA | |
| gACA36- | UUCCAAUGgUggUgUUUCAGUCCAGGGCAGCUUCCCUGUUCUGAccU | 80 |
| CBS- | gUcgAAUUGGGACAUUAAAAUCGGCUGGUgUagUgUUGGACUAAGAA | |
| W390X | AGUAUUAUUCAUAGUCCccUgUcgACCAGCCUACAAAA | |
| gACA19- | GUGCACAUUUcggccUGACCUGCUUUCUUCUAUGUGAGUAGUGUUUc | 81 |
| PCCB- | UagUgaUGUGCUAUACAAAUAAUUGAAGUUcggccUGCAGUAUAACU | |
| R111X | AUAAAUAGUAAUGCUGCUcUagUACUUCAGACAAAA | |
| gACA36- | UUCCAAAUAUcggccUUCAGUCCAGGGCAGCUUCCCUGUUCUGAUUU | 82 |
| PCCB- | agUUCUUUGGAACAUUAAAAUCGGCUGGAAUcggccUGGACUAAGAA | |
| R111X | AGUAUUAUUCAUAGUCCUUcagUgUCCAGCCUACAAAA | |
| gACA19- | GUGCACAUcUggUUgUGACCUGCUUUCUUCUAUGUGAGUAGUGUCUa | 83 |
| PEX7- | UaUUGAUGUGCUAUACAAAUAAUUGAAGGUggUUgUGCAGUAUAACU | |
| R232X | AUAAAUAGUAAUGCUGCUaUaUUUcU | |
| | UCAGACAAAA | |
| gACA36- | UUCCAAAUcUggUUgUUCAGUCCAGGGCAGCUUCCCUGUUCUGAUaU | 84 |
| PEX7- | aUUUcUUUGGGACAUUAAAAUCGGCUGGUcUggUUgUGGACUAAGAA | |
| R232X | AGUAUUAUUCAUAGUCCUaUgUUUACCAGCCUACAAAA | |
| gACA19- | GUGCACAUCCAUUAGUGCCCUGCUUUCUUCUAUGUGAGUAGUGGUGG | 181 |
| LMNA- | UCUCGAUGUGCUAUACAAAUAAUUGAAGGCAUUAGUGGAGUAUAACU | |
| R225X | AUAAAUAGUAAUGCUUUGGUCUCCCUUCAGACAAAA | |
| gACA36- | UUCCAGGUCCAUUAGUGCGGUCCAGGGCAGCUUCCCUGUUCUGUGGU | 182 |
| LMNA- | CUCAUCCUGGGACAUUAAAAUCGGCUGGUCCAUUGGUUGACUAAGAA | |
| R225X | AGUAUUAUUCAUAGUUGGGUCUCAACCAGCCUACAAAA | |
| gACA19- | GUGCACAUCGAGUAGCGACCUGCUUUCUUCUAUGUGAGUAGUGUUUC | 183 |
| F9-Y22X | CUGAGAUGUGCUAUACAAAUAAUUGAAGCGAGUAGCGCAGUAUAACU | |
| | AUAAAUAGUAAUGCUGCUCCUGAGCUUCAGACAAAA | |
| gACA36- | UUCCAAAGUGAGUAGCUCAGUCCAGGGCAGCUUCCCUGUUCUGAUCC | 184 |
| F9-Y22X | UGAAAUUUGGGACAUUAAAAUCGGCUGGUUGAGUAGCGGACUAAGAA | |
| | AGUAUUAUUCAUAGUCCUCCUGAAAACCAGCCUACAAAA | |
| gACA19- | GUGCACAUCUAGAUAUGACCUGCUUUCUUCUAUGUGAGUAGUGUUUA | 185 |
| F9-G21X | AAAUGAUGUGCUAUACAAAUAAUUGAAGCUAGAUAUGCAGUAUAACU | |
| | AUAAAUAGUAAUGCUGCUAAAAUGCUUCAGACAAAA | |
| gACA36- | UUCCAAAGGUAGAUAUUCAGUCCAGGGCAGCUUCCCUGUUCUGAUAA | 186 |
| F9-G21X | AAGGAUUUGGGACAUUAAAAUCGGCUGGUGUAGAUAUGGACUAAGAA | |
| | AGUAUUAUUCAUAGUCCUAAAAGGAACCAGCCUACAAAA | |
| gACA19- | GUGCACAUCUAUCCUUGACCUGCUUUCUUCUAUGUGAGUAGUGUUUG | 187 |
| ABCA4- | CUGCGAUGUGCUAUACAAAUAAUUGAAGCUAUCCUUGCAGUAUAACU | |
| R408X | AUAAAUAGUAAUGCUGCUGCUGCGCUUCAGACAAAA | |
| gACA36- | UUCCAAAGAUAUCCUUUCAGUCCAGGGCAGCUUCCCUGUUCUGAUGC | 188 |
| ABCA4- | UGCAGUUUGGGACAUUAAAAUCGGCUGGUGUAUCCUUGGACUAAGAA | |
| R408X | AGUAUUAUUCAUAGUCCUGCUGCAGACCAGCCUACAAAA | |
| gACA19- | GUGCACAUCCUUGUGCGACCUGCUUUCUUCUAUGUGAGUAGUGUCUG | 189 |
| RS1-Y65X | GGUUGAUGUGCUAUACAAAUAAUUGAAGGCUUGUGCGCAGUAUAACU | |
| | AUAAAUAGUAAUGCUGCUGGGUUCCUUCAGACAAAA | |
| gACA36- | UUCCAGAUCCUUGUGCUGAGUCCAGGGCAGCUUCCCUGUUCUCAUGG | 190 |
| RS1-Y65X | GUAACUCUGGGACAUUAAAAUCGGCUGGUGUUUGUGCGGACUAAGAA | |
| | AGUAUUAUUCAUAGUCCUGGGUGAACCAGCCUACAAAA | |
| gACA19- | GUGCACAUGGUUACAUGACCUGCUUUCUUCUAUGUGAGUAGUGUUGA | 191 |
| Rpe65- | GGAGAUAUGUGCUAUACAAAUAAUUGAAGGUUUACAUGCAGUAUAAC | |
| R44X | UAUAAAUAGUAAUGCUGUGAGGGUCCUUCAGACAAAA | |
| gACA36- | UUCCAAAGACUUACAUGCAGUCCAGGGCAGCUUCCCUGUUCUGUGAG | 192 |
| Rpe65- | GGCGAUUUGGGACAUUAAAAUCGGCUGGUGUUUACAUGGACUAAGAA | |
| R44X | AGUAUUAUUCAUAGUCUGAGGGAAACCAGCCUACAAAA | |

In one aspect, the present inventors discovered that the editing efficiency of a gsnoRNA was surprisingly higher when the gsnoRNA was encoded in tandem with its target RNA. Example 3 provides results demonstrating the increased editing efficiency using a reporter construct encoding a gsnoRNA and a target RNA in tandem.

Thus, in some aspects, provided herein is a nucleic acid molecule comprising a nucleotide sequence encoding the guide small nucleolar RNA (gsnoRNA) in tandem with a nucleotide sequence encoding the target RNA. In some embodiments, the nucleotide sequence encoding the gsnoRNA is driven by a U6 or U1 promoter. In some embodiments, the nucleotide sequence encoding the target RNA is driven by the same or a different promoter. In some embodiments, the gsnoRNA encoded in tandem with a nucleotide sequence encoding the target RNA provides at least 1.1-, 1.2-, 1.3-, 1.4-, 1.5-, 1.6-, 1.7-, 1.8-, 1.9-, or 2-fold greater editing efficiency of the target RNA compared to the same gsnoRNA encoded in a separate nucleic acid molecule from the target RNA.

### III. Methods

In some embodiments, provided herein is a method for editing a target RNA in a host cell, comprising introducing an engineered guide small nucleolar RNA (gsnoRNA) and a nucleic acid molecule encoding a DKC1 protein into the host cell, wherein the gsnoRNA comprises a guide sequence that hybridizes to a sequence comprising a target uridine residue in the target RNA, and wherein the gsnoRNA recruits the DKC1 protein to modify the target uridine residue into a pseudouridine residue in the target RNA. In some embodiments, the DKC1 protein is a DKC1 isoform (e.g., isoform 3) with cytoplasmic localization. In some embodiments, the DKC1 protein is part of a ribonucleoprotein (RNP) complex that associates with the gsnoRNA. In some embodiments, the DKC1 protein comprises a deletion of a nuclear localization signal (NLS) relative to a wildtype DKC1 protein of the same species. In some embodiments, the DKC1 protein is a truncated DKC1 variant or a DKC1 variant comprising a deletion, such as any of the truncation or deletion variants described in Section II A above. In some embodiments, the gsnoRNA recruits NOP10, GAR1, and NHP2 in the host cell.

In some embodiments, the method comprises introducing a nucleic acid (e.g., a nucleic acid vector) encoding the gsnoRNA into the cell. In other embodiments, the method comprises introducing a gsnoRNA oligonucleotide into the cell. In some embodiments, the gsnoRNA comprises a first hairpin and H box and a second hairpin and ACA box. In some embodiments, the gsnoRNA is prepared by in vitro transcription. In some embodiments, the gsnoRNA prepared by in vitro transcription comprises the sequence of any one of SEQ ID NOs: 4-6, 9-12, 15-19 and 22-36. In some embodiments, the gsnoRNA prepared by in vitro transcription comprises a 5′ cap modification or a 5′ hairpin (e.g., of a U6+U27 expression cassette). In some embodiments, the gsnoRNA prepared by in vitro transcription comprises a 5′ cap modification. In some embodiments, the 5′ cap modification is is a m⁷G modification (e.g., a cap 0, cap 1, or cap 2 modification) or an m⁶Aₘ modification. Suitable methods for adding a 5′ cap to an RNA oligonucleotide have been described, for example, in U.S. Pat. No. 10,494,399, the contents of which are herein incorporated by reference in their entirety. In some embodiments, the gsnoRNA further comprises a 3′ hairpin (e.g., the gsnoRNA comprises the sequence of any one of SEQ ID NOs: 4-6, 9-12, 15-19 and 22-36 and a 3′ hairpin). In some embodiments, the gsnoRNA comprises a 5′ cap modification and does not comprise a 3′ hairpin (e.g., as shown in FIG. 15A). In some embodiments, the in vitro transcripbed gsnoRNA is capable of guiding targeted pseudouridylation in the cell. In some embodiments, the 5′ cap modification is introduced by in vitro transcription using an m⁷G(5′)ppp(5′)G cap analog.

In some embodiments, the method comprises introducing a nucleic acid (e.g., a nucleic acid vector) encoding a gsnoRNA that is a half gsnoRNA (e.g., comprising a single hairpin and an H box, or comprising a single hairpin and an ACA box) into a cell. In other embodiments, the method comprises introducing a gsnoRNA that is a half gsnoRNA (e.g., comprising a single hairpin and an H box, or comprising a single hairpin and an ACA box) into a cell. In some embodiments, the gsnoRNA comprises no more than 20, no more than 15, no more than 10, no more than 8, no more than 6, or no more than 4 2′-OMe or 2′-MOE modifications. In some embodiments, the gsnoRNA comprises between about 2 and about 6 2′-OMe or 2′-MOE modifications. In some embodiments, the gsnoRNA comprises about 4 2′-OMe or 2′-MOE modifications. In some embodiments, the gsnoRNA comprises no more than 5 modified sugars. In some embodiments, the gsnoRNA comprises two nucleosides comprising modified sugar moieties (e.g., 2′-OMe) at the 5′ end and two nucleosides comprising modified sugar moieties (e.g., 2′-OMe) at the 3′ end of the gsnoRNA. In some embodiments, the gsnoRNA comprises no more than four, three, or two nucleosides comprising modified sugar moieties (e.g., 2′-OMe) at the 5′ end and no more than four, three, or two nucleosides comprising modified sugar moieties (e.g., 2′-OMe) at the 3′ end of the gsnoRNA. In some embodiments, the gsnoRNA comprises one or more phosphorothioate inter-nucleosidic linkages. In some embodiments, the gsnoRNA comprises no more than 20, no more than 15, no more than 10, no more than 8, or no more than 6 phosphorothioate linkages. In some embodiments, the gsnoRNA comprises between about 2 and about 10 phosphorothioate linkages. In some embodiments, the gsnoRNA comprises about 6 phosphorothioate linkages. In some embodiments, the gsnoRNA comprises about three phosphorothioate linkages at the 5′ end and about three phosphorothioate linkages at the 3′ end of the gsnoRNA. In some embodiments, the gsnoRNA comprises no more than five, four, or three phosphorothioate linkages at the 5′ end and no more than five, four, or three phosphorothioate linkages at the 3′ end of the gsnoRNA. In some embodiments, the gsnoRNA comprises a 5′ hairpin, an H box (consensus sequence ANANNA), a 3′ hairpin, and an ACA box (consensus sequence ANA). In some embodiments, the gsnoRNA comprises a single hairpin and an H box (referred to herein as a gH5 or H5 for 5′ half gsnoRNA encoding sequence or gsnoRNA oligonucleotide, respectively), and lacks an ACA box. In some embodiments, the gsnoRNA comprises a single hairpin and an ACA box (referred to herein as a gH3 or rH3 for 3′ half gsnoRNA encoding sequence or gsnoRNA oligonucleotide, respectively), and lacks an H box. In some embodiments, the gsnoRNA comprising a single hairpin is between 60 and 70 nucleotides in length. In some embodiments, the gsnoRNA comprising a single hairpin is about 65 nucleotides in length.

The present disclosure is exemplified by, but not limited to, reversing the effect of nonsense stop mutations that usually lead to translation termination and mRNA degradation (via Nonsense Mediated Decay, see below). In another aspect, targeted pseudouridylation can act as a means to recode uridine-containing codons as a mean to modulate protein function via amino acid substitution, for instance in crucial protein regions such as protein kinase active centers.

One of the consequences of mutations leading to PTCs in the coding sequence of a gene is the decrease of the mRNA levels. This is due to a mechanism known as the Nonsense-Mediated Decay (NMD), which is a cellular surveillance mechanism that degrades aberrant mRNA transcripts, preventing transcripts that were not correctly processed from being translated. It is estimated that one-third of genetic disorders are a result of a mutation leading to a PTC (such as for instance in CF, retiniti pigmentosa (RP), and beta-thalassemia). In a normal scenario, exon-junction complexes (EJCs) are formed during splicing. Then, during the first translation round, ribosomes displace these EJCs. On the other hand, when a PTC is located more than 50-54 nucleotides upstream of the last EJC, the NMD pathway is triggered by formation of a termination complex consisting of EJC-associated NMD factors. When this happens during the first pioneer round of translation and the ribosomes co-exist with at least one EJC downstream their location, this triggers the de capping and 5′-to-3′ exonuclease activity and also de-adenylation of the tail and 3′-to-5′ exonuclease-mediated transcript decay. In order to tackle the aforementioned genetic disorders, or any disorder that is due to a similar mutation, inhibition of this pathway in a gene-specific and sequence-specific manner is therefore crucial.

In some aspects, provided herein are methods for recoding a PTC, which results in an increase of mRNA levels, and in translational read-though of the recoded mRNA into a full-length protein. In some embodiments, the methods and compositions provided herein allow for PTC read-through of more than 4%, more than 5%, more than 10%, more than 12%, more than 15%, more than 20%, or more than 30%. In some embodiments, the methods and compositions herein allow for suppression of nonsense-mediated decay (NMD) by more than 10%, more than 12%, more than 15%, more than 20%, or more than 30%. PTC read-through can be assayed by evaluating protein levels, either by directly quantifying the protein expression or by assaying an activity of the expressed protein. Methods for assessing NMD suppression are also known in the art. For example, to assess NMD suppression, a known NMD-inhibition reporter assay (Zhang et al. 1998, RNA 4(7):801-815) can be used, and translational read-through of a gene carrying a PTC can also be assessed. As exemplified herein, fluorescent reporter genes carrying nonsense mutations were used as the target sequence. Without correction, this nonsense mutation leads to a lower abundance of mRNA (as a result of NMD) as well as to a truncated protein, resulting in the absence of fluorescent signal. As shown herein, correction of the mutation via targeted pseudouridylation allows the full length protein to be translated from the mRNA. The skilled person understands that the PTC region of the fluorescent reporter constructs described herein can be exchanged by any other model or therapeutically relevant target RNA of interest.

In some embodiments, provided herein are methods for recoding a PTC in an RNA encoding a protein, wherein the method results in expression of the full-length protein in the host cell of at least 4% (e.g., at least 5%, at least 6%, at least 7%, at least 8%, at least 9% or at least 10%) of the expression level of the full-length protein without a premature termination codon. In some embodiments, the method results in expression of the full-length protein, wherein the expression of the protein is detectable without enrichment (e.g., without enrichment by immunoprecipitation). In some embodiments, the protein is detected via a tag (e.g., via a fluorescent tag). In some embodiments, the protein is detected by immo-staining according to methods known in the art. In some embodiments, the method results in expression of the full-length protein in at least 20% of host cells (e.g., at least 25%, at least 30%, at least 35%, at least 40%, at least 45%, or at least 50% of host cells).

In some aspects, provided herein are methods for treating, preventing, and/or blocking nonsense-mediated RNA decay of a target mRNA, the methods comprising introducing a guide small nucleolar RNA (gsnoRNA) and a nucleic acid molecule encoding a DKC1 protein into the host cell, wherein the gsnoRNA comprises a guide sequence that hybridizes to a premature termination codon (PTC) sequence comprising a target uridine residue in the target mRNA, and wherein the gsnoRNA recruits the DKC1 protein to modify the target uridine residue into a pseudouridine residue in the target RNA, whereby the pseudouridylation of the target uridine promotes read-through of the PTC. In some embodiments, the DKC1 protein is a DKC1 isoform with cytoplasmic localization. In some embodiments, the DKC1 protein is part of a ribonucleoprotein (RNP) complex that associates with the gsnoRNA. In some embodiments, the DKC1 protein comprises a deletion of a nuclear localization signal (NLS) relative to a wildtype DKC1 protein of the same species. In some embodiments, the DKC1 protein is a truncated DKC1 variant or a DKC1 variant comprising a deletion, such as any of the truncation or deletion variants described in Section II A above.

In some embodiments, the DKC1 protein is an endogenous protein of the host cell. In some embodiments, the DKC1 protein is an endogenous, naturally expressed DKC1 isoform of the host cell, wherein the DKC1 isoform has cytoplasmic localization in the host cell. In some embodiments, the DKC1 protein corresponds to isoform 2 of a human DKC1 protein.

In some embodiments, the DKC1 and snoRNA can be delivered into the cell together (e.g., as part of a ribonucleoprotein (RNP) complex). In some embodiments, the snoRNP comprises the gsnoRNA and DKC1, NHP2, GAR1, and/or NOP10.

In some embodiments, provided herein is a method for editing a target RNA in a host cell, comprising introducing an engineered gsnoRNA into the host cell, wherein the gsnoRNA comprises a guide sequence that hybridizes to a sequence comprising a target uridine residue in the target RNA (e.g., mRNA), wherein the host cell expresses a DKC1 isoform with cytoplasmic localization, and wherein the gsnoRNA recruits the DKC1 isoform to modify the target uridine residue into a pseudouridine residue in the target RNA. In some embodiments, the method comprises introducing a splice-switching antisense oligonucleotide (ASO) into the host cell, wherein the ASO enhances expression of a DKC1 protein that is an endogenous DKC1 isoform with cytoplasmic localization in the host cell.

Splice-switching antisense oligonucleotides (ASOs) alter splicing by directing splice site selection. Splice-switching ASOs can modulate pre-mRNA splicing by binding to target pre-mRNAs and blocking access of the splicing machinery to a particular splice site, and can be used to produce novel splice variants, correct aberrant splicing or manipulate alternative splicing. Methods for the design and delivery of splice-switching antisense oligonucleotides to cells have been described, for example in U.S. Patent Publications US20180334677 and US20120040917, U.S. Pat. No. 10,190,117, and Disterer et al. Hum Gene Ther. 2014 Jul; 25(7):587-98, the contents of which are herein incorporated by reference in their entirety.

In some embodiments, the splice-switching ASO binds to a pre-mRNA of a DKC1 gene and directs splicing of DKC1 isoform 3. In some embodiments, introducing the splice-switching ASO increases expression of a DKC1 protein that is an endogenous DKC1 isoform with cytoplasmic localization in the host cell by at least 1.1-, 1.2-, 1.3-, 1.4-, 1.5-, 1.6-, 1.7-, 1.8-, 1.9-, 2-, 3-, 4-, 5-, or 10-fold compared to the expression of the same isoform in the host cell in the absence of the ASO. In some embodiments, administering the splice-switching ASO increases expression of DKC1 isoform 3 by at least 1.1-, 1.2-, 1.3-, 1.4-, 1.5-, 1.6-, 1.7-, 1.8-, 1.9-, 2-, 3-, 4-, 5-, or 10-fold compared to the expression of DKC1 isoform 3 in the host cell in the absence of the ASO.

In some embodiments, the splice-switching ASOs may be delivered via aptamers, Inverse Molecular Sentinel nanoprobes, ASO encapsulated liposome-DNA-polycation or ASO encapsulated liposome-protamine-hyluronic acid nanoparticles and the like. Suitable methods of delivering aptamers can be found in Kotula, J. W., et al., Aptamer-mediated delivery of splice-switching oligonucleotides to the nuclei of cancer cells. Nucleic Acid Ther, 2012. 22(3): p. 187-95, the contents of which are incorporated by reference in their entirety.

In some embodiments, provided herein is a method for editing a target RNA in a host cell, comprising introducing an engineered gsnoRNA into the host cell, wherein the gsnoRNA comprises a guide sequence that hybridizes to a sequence comprising a target uridine residue in the target RNA, wherein the gsnoRNA comprises a scaffold sequence derived from wildtype ACA19, ACA44, ACA27, E2, ACA3, ACA17, ACA2b or ACA36, and wherein the gsnoRNA recruits a DKC1 protein in the host cell to modify the target uridine residue into a pseudouridine residue in the target RNA (e.g., mRNA). In some embodiments, the gsnoRNA comprises a scaffold sequence derived from wildtype ACA2b or ACA36. In some embodiments, the DKC1 protein is a DKC1 isoform with cytoplasmic localization. In some embodiments, the DKC1 protein is part of a ribonucleoprotein (RNP) complex that associates with the gsnoRNA.

In some embodiments, provided herein is a method for editing a target RNA in a host cell, comprising introducing an engineered gsnoRNA into the host cell, wherein the gsnoRNA comprises a guide sequence that hybridizes to a sequence comprising a target uridine residue in the target RNA, wherein the gsnoRNA comprises a scaffold sequence derived from wildtype ACA19, ACA44, ACA27, E2, ACA3, ACA17, ACA2b or ACA36, and wherein the gsnoRNA recruits a DKC1 protein in the host cell to modify the target uridine residue into a pseudouridine residue in the target RNA (e.g., mRNA). The engineered gsnoRNA can be any one of the engineered gsnoRNAs described in Section II B. In some embodiments, the gsnoRNA comprises a nucleotide sequence selected from the group consisting of SEQ ID NOs: 4-6, 9-12, 15-19, 22-36, and 177-179. In some embodiments, the DKC1 protein is a DKC1 isoform with cytoplasmic localization. In some embodiments, the DKC1 protein is part of a ribonucleoprotein (RNP) complex that associates with the gsnoRNA. In some embodiments, the DKC1 protein comprises a deletion of a nuclear localization signal (NLS) relative to a wildtype DKC1 protein of the same species. In some embodiments, the DKC1 protein is a truncated DKC1 variant or a DKC1 variant comprising a deletion, such as any of the truncation or deletion variants described in Section II A above.

In some embodiments, provided herein is a method for editing a target RNA in a host cell, comprising introducing an engineered gsnoRNA into the host cell, wherein the gsnoRNA comprises a guide sequence that hybridizes to a sequence comprising a target uridine residue in the target RNA, wherein the gsnoRNA comprises a nucleotide sequence selected from the group consisting of SEQ ID NOs: 4-6, 9-12, 15-19, 22-36, and 177-179, and wherein the gsnoRNA recruits a DKC1 protein in the host cell to modify the target uridine residue into a pseudouridine residue in the target RNA. In some embodiments, the DKC1 protein is a DKC1 isoform with cytoplasmic localization. In some embodiments, the DKC1 protein comprises a DKC1 protein fragment corresponding to amino acid residues 1 to 419 of a full-length human DKC1 protein, wherein the amino acid numbering is according to SEQ ID NO: 1. In some embodiments, the DKC1 protein is part of a ribonucleoprotein (RNP) complex that associates with the gsnoRNA. In some embodiments, the DKC1 protein comprises a deletion of a nuclear localization signal (NLS) relative to a wildtype DKC1 protein of the same species. In some embodiments, the DKC1 protein is a truncated DKC1 variant or a DKC1 variant comprising a deletion, such as any of the truncation or deletion variants described in Section II A above.

In some embodiments, the methods provided herein comprise introducing a nucleic acid molecule comprising a nucleotide sequence encoding the guide small nucleolar RNA (gsnoRNA) in tandem with a nucleotide sequence encoding the target RNA into a host cell. In some embodiments, the nucleotide sequence encoding the gsnoRNA is driven by a U6 or U1 promoter. In some embodiments, the nucleotide sequence encoding the target RNA is driven by the same or a different promoter. In some embodiments, the gsnoRNA encoded in tandem with a nucleotide sequence encoding the target RNA provides at least 1.1-, 1.2-, 1.3-, 1.4-, 1.5-, 1.6-, 1.7-, 1.8-, 1.9-, or 2-fold greater editing efficiency of the target RNA compared to the same gsnoRNA encoded in a separate nucleic acid molecule from the target RNA.

In some embodiments, the methods provided herein comprise introducing the guide small nucleolar RNA (gsnoRNA) to an endogenous nucleic acid molecule of a host cell, wherein the endogenous nucleic acid molecule comprises a nucleotide sequence encoding the target RNA. In some embodiments, the introducing comprises inserting a nucleotide sequence encoding the gsnoRNA into a region of the endogenous nucleic acid molecule that is directly or indirectly adjacent to the region encoding the target RNA. In some embodiments, the nucleotide sequence encoding the gsnoRNA is driven by a U6 or U1 promoter. Methods for inserting nucleotide sequences into endogenous nucleic acid molecules are known in the art, such as guided-nuclease (e.g., CRISPR/Cas) editing and homology-directed repair. In some embodiments, the gsnoRNA inserted into a region of an endogenous nucleic acid molecule that is directly or indirectly adjacent to a nucleotide sequence encoding the target RNA provides at least 1.1-, 1.2-, 1.3-, 1.4-, 1.5-, 1.6-, 1.7-, 1.8-, 1.9-, or 2-fold greater editing efficiency of the target RNA compared to the same gsnoRNA encoded in a separate nucleic acid molecule from the target RNA.

In some embodiments, the degree of recruiting and redirecting the pseudouridylation entities resident in the cell may be regulated by the dosing and the dosing regimen of the gsnoRNA. This is something to be determined by the experimenter (e.g., in vitro) or the clinician, usually in phase I and/or II clinical trials.

In some embodiments, the methods provided herein comprise modification of target RNA (e.g., mRNA) sequences in eukaryotic, (e.g., metazoan or mammalian cells, such as human cells). In some aspects, the methods and compositions provided herein can be used with cells from any organ e.g. skin, lung, heart, kidney, liver, pancreas, gut, muscle, gland, eye, brain, blood and the like. The cell can be located in vitro or in vivo. One advantage of the methods, compositions, systems, kits, and articles of manufacture of the present disclosure is that they can be used with cells in situ in a living organism, but can also be used with cells in culture. In some embodiments cells are treated ex vivo and are then introduced into a living organism (e.g. re-introduced into an organism from whom they were originally derived). The methods, compositions, systems, kits, and articles of manufacture of the present disclosure can also be used to edit target RNA sequences in cells within a so-called organoid. Organoids can be thought of as three-dimensional in vitro-derived tissues but are driven using specific conditions to generate individual, isolated tissues (e.g. see Lancaster and Knoblich. 2014, Science 345 (6194):1247125). In a therapeutic setting they are useful because they can be derived in vitro from a patient's cells, and the organoids can then be re-introduced to the patient as autologous material, which is less likely to be rejected than a normal transplant. The cell to be treated will generally have a genetic mutation. The mutation may be heterozygous or homozygous. In some embodiments, the methods and compositions provided herein can be used to modify point mutations. In some embodiments, the methods and compositions provided herein are suitable for modifying sequences in cells, tissues or organs implicated in a diseased state of a subject (e.g., a human subject), for instance when the human subject suffers from a disease associated with a PTC.

The present disclosure provides methods that can be used to make a change (pseudouridylation) in a target RNA sequence in a eukaryotic cell through the use of an oligonucleotide (e.g., any of the gsnoRNAs described in Section II B above, or any gsnoRNA based on the engineered scaffolds described in Section II B above) that is capable of targeting a site to be edited and recruiting RNA editing proteins (e.g., DKC1) to bring about the editing reaction(s). In some embodiments, the DKC1 is endogenous DKC1. In some embodiments, the DKC1 is exogenously delivered. In some embodiments, the method comprises increasing the relative proportion of DKC1 isoform 3 or a DKC1 protein with cytoplasmic localization. The target RNA sequence may comprise a mutation that one may wish to correct or alter, such as a point mutation (a transition or a trans version). The target RNA may be any cellular or viral RNA sequence, but is more usually a pre-mRNA or an mRNA with a protein coding function. In some embodiments, the target sequence is endogenous to the eukaryotic, (e.g., mammalian, e.g., human) cell.

In some embodiments, the methods provided herein are suitable for promoting read-through of a PTC, wherein the PTC is an opal codon (UGA), an amber codon (UAG), or an ochre codon (UAA). In some embodiments, the PTC is an opal codon, and the method results at least 10%, at least 15%, at least 20%, or at least 25% read-through efficiency, wherein read-through efficiency is assayed as the percent of protein expression or activity (e.g., fluorescent intensity) compared to a control that lacks the PTC. In some embodiments, the PTC is an amber codon (UAG), and the method results in at least 2%, at least 5%, at least 10%, at least 12%, or at least 14% read-through efficiency, wherein read-through efficiency is assayed as the percent of protein expression or activity (e.g., fluorescent intensity) compared to a control that lacks the PTC. In some embodiments, the method results in at least 20%, 25%, 30%, 35%, 40%, 45%, 50%, 55%, 60%, 65%, or 70% of cells expressing detectable levels of the full-length protein encoded by a target gene comprising the PTC.

In some embodiments, the target uridine in the target RNA is a premature termination codon in a sequence encoding a protein, wherein the method results in expression of the full-length protein in the host cell at at least 4% (e.g., at least 5%, at least 6%, at least 7%, at least 8%, at least 9%, at least 10%, or higher) of the expression level of the full-length protein without a premature termination codon.

In some embodiments, the target uridine in the target RNA is a premature termination codon in a sequence encoding a protein, wherein the method results in expression of the full-length protein, and wherein the expression of the protein is detectable without enrichment (e.g., without enrichment by immunoprecipitation). In some embodiments, the protein is detected via a tag (e.g., via a fluorescent tag). In some embodiments, the protein is detected by immo-staining according to methods known in the art.

In some embodiments, the target uridine in the target RNA is a premature termination codon in a sequence encoding a protein, wherein the method results in expression of the full-length protein in at least 20% of host cells, e.g., at least 25%, at least 30%, at least 35%, at least 40%, at least 45%, at least 50%, or higher percentage of host cells.

Also provided are engineered gsnoRNA compositions or engineered RNA editing systems described herein for use in any one of the methods described herein, such as a method of editing a target RNA or a method of treatment. Use of any one of the engineered gsnoRNA compositions or engineered RNA editing systems described herein in the preparation of a medicament for treating a disease or condition.

### A. Methods of Treatment

In some aspects, the methods provided herein comprise modifying a target RNA using a gsnoRNA that recruits a DKC1 protein to modify the target RNA. In some embodiments, the gsnoRNA hybridizes to a target a sequence comprising a target uridine residue, and the modification of the RNA comprises modification of the target uridine to pseudouridine.

In some embodiments, the target RNA is an endogenous RNA of a cell (e.g., a eukaryotic cell such as a mammalian or human cell). In some embodiments, the target RNA is an endogenously transcribed RNA of the cell (e.g., transcribed from an endogenous nucleic acid sequence of the cell). In some embodiments, the target RNA is transcribed from a nucleic acid sequence that has been introduced into the cell (e.g., an RNA transcribed from an exogenously added nucleic acid molecule). In some embodiments, the target RNA is a ribosomal RNA. In some embodiments, the target RNA is a messenger RNA (mRNA).

In some embodiments, the sequence comprising the target uridine in the target RNA is a stop codon, and modification of the target uridine to pseudouridine causes the stop codon to be translated as a coding codon. In some embodiments, the stop codon is a premature termination codon (PTC). In some embodiments, the PTC is associated with a genetic disease or condition. Converting the target uridine in such a PTC to a pseudouridine, by using the means and methods of the present disclosure, then results in proper read-through of the reading frame during translation, thereby providing a (partly or fully) functional full length protein.

In some embodiments, provided herein is a method of treating a disease or condition associated with a PTC in a target RNA in a subject, comprising editing the target RNA in a cell of the subject using any of the RNA editing methods described herein, wherein the gsnoRNA comprises a guide sequence that hybridizes to the PTC in the target RNA, and wherein modification of the uridine residue in the PTC to a pseudouridine residue causes translation read-through of the PTC in the target RNA, thereby treating the disease or condition in the subject.

In some embodiments, the method of treating a disease or condition associated with a PTC in a target RNA in a subject comprises introducing an engineered gsnoRNA into a host cell of the subject, wherein the gsnoRNA comprises a guide sequence that hybridizes to the PTC comprising the uridine residue in the target RNA, wherein the gsnoRNA comprises a scaffold sequence derived from wildtype ACA2b, ACA36, ACA44, ACA27, E2, ACA3, or ACA17, and wherein the gsnoRNA recruits a DKC1 protein in the host cell to modify the target uridine residue into a pseudouridine residue in the target RNA. In some embodiments, the DKC1 protein is an endogenous DKC1 protein of the host cell. In some embodiments, the method further comprises introducing a nucleic acid encoding the DKC1 protein into the host cell. In some embodiments, the DKC1 protein has cytoplasmic localization in the host cell. In some embodiments, the DKC1 protein comprises a DKC1 protein fragment corresponding to amino acid residues 41 to 420 of a human DKC1 isoform 3 protein, wherein the amino acid numbering is according to SEQ ID NO: 2. In some embodiments, the DKC1 protein comprises an amino acid sequence having at least 85% (e.g., at least about any one of 90%, 91%, 92%, 93%, 94%, 95%, 96%, 97%, 98%, 99%, or more) identity to SEQ ID NO: 88. In some embodiments, the DKC1 protein comprises the amino acid sequence of SEQ ID NO: 88.

In some embodiments, the method of treating a disease or condition associated with a PTC in a target RNA in a subject comprises introducing an engineered gsnoRNA into a host cell of the subject, wherein the gsnoRNA comprises a guide sequence that hybridizes to the PTC comprising the target uridine residue in the target RNA, wherein the gsnoRNA comprises a nucleotide sequence selected from the group consisting of SEQ ID NOs: 4-6, 9-12, 15-19, 22-36, and 177-179, and wherein the gsnoRNA recruits a DKC1 protein in the host cell to modify the target uridine residue into a pseudouridine residue in the target RNA. In some embodiments, the DKC1 protein is an endogenous DKC1 protein of the host cell. In some embodiments, the method further comprises introducing a nucleic acid encoding the DKC1 protein into the host cell. In some embodiments, the DKC1 protein has cytoplasmic localization in the host cell. In some embodiments, the DKC1 protein comprises a DKC1 protein fragment corresponding to amino acid residues 41 to 420 of a human DKC1 isoform 3 protein, wherein the amino acid numbering is according to SEQ ID NO: 2. In some embodiments, the DKC1 protein comprises an amino acid sequence having at least 85% (e.g., at least about any one of 90%, 91%, 92%, 93%, 94%, 95%, 96%, 97%, 98%, 99%, or more) identity to SEQ ID NO: 88. In some embodiments, the DKC1 protein comprises the amino acid sequence of SEQ ID NO: 88.

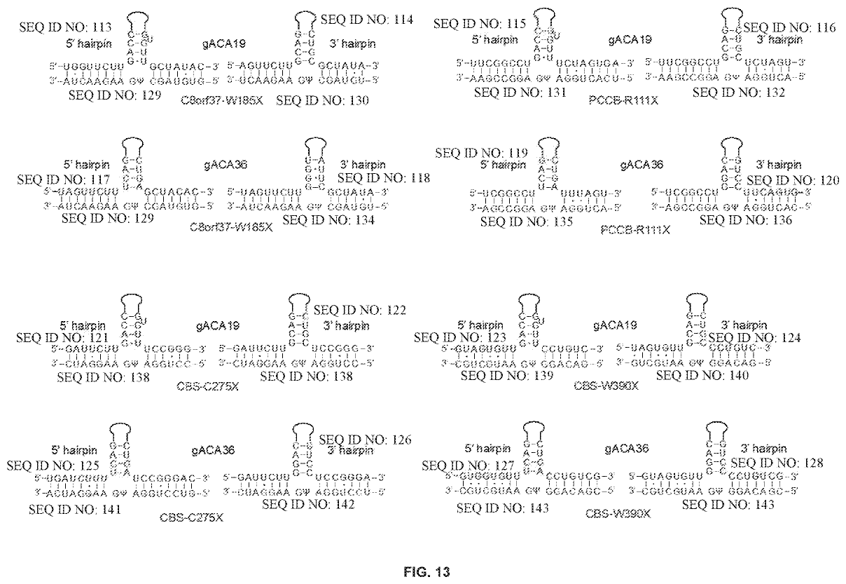

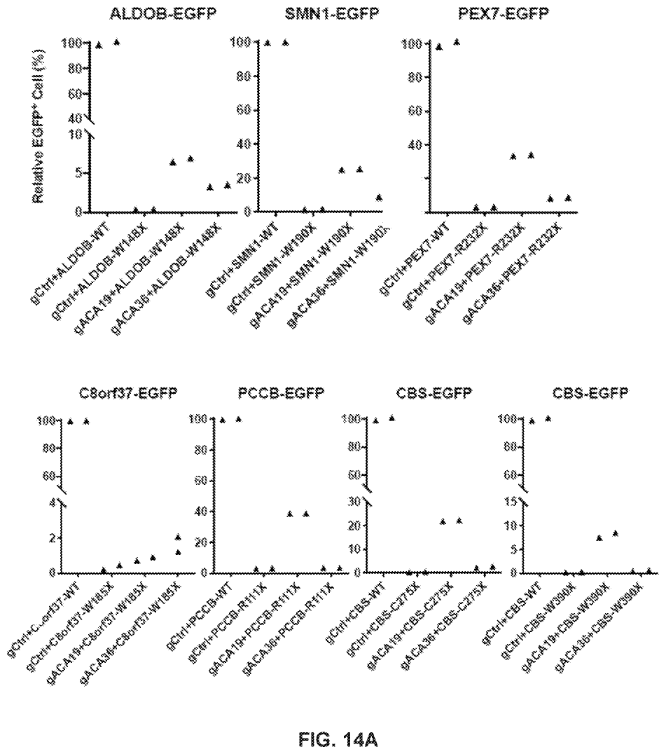

In some embodiments, the gsnoRNA is a half gsnoRNA, e.g., comprising a single hairpin and an H box, or a single hairpin and an ACA box. In some embodiments, the gsnoRNA comprises or consists of any one of the sequences set forth in SEQ ID NOS: 89-100 and 113-128, as shown in FIG. 12B and FIG. 13.

In some embodiments, the method of treating a disease or condition associated with a PTC in a target RNA in a subject comprises introducing an engineered gsnoRNA into a host cell of the subject, wherein the gsnoRNA comprises a sequence selected from SEQ ID NOs: 71-84. Sequences of exemplary engineered gsnoRNAs targeting uridine residues of exemplary disease-associated PTCs are shown in Table 4.

In some embodiments the method of treating a disease or condition associated with a PTC in a target RNA in a subject comprises introducing (a) an engineered gsnoRNA and (b) a splice-switching antisense oligonucleotide (ASO) into a host cell of the subject, wherein the gsnoRNA comprises a guide sequence that hybridizes to a sequence comprising a target uridine residue in the target RNA, wherein the ASO enhances expression of a DKC1 protein that is an endogenous DKC1 isoform with cytoplasmic localization in the host cell, and wherein the gsnoRNA recruits the DKC1 protein to modify the target uridine residue into a pseudouridine residue in the target RNA. In some embodiments, the splice-switching ASO binds to a pre-mRNA of a DKC1 gene and directs splicing of DKC1 isoform 3. In some embodiments, introducing the splice-switching ASO increases expression of a DKC1 protein that is an endogenous DKC1 isoform with cytoplasmic localization in the host cell by at least 1.1-, 1.2-, 1.3-, 1.4-, 1.5-, 1.6-, 1.7-, 1.8-, 1.9-, 2-, 3-, 4-, 5-, or 10-fold compared to the expression of the same isoform in the host cell in the absence of the ASO. In some embodiments, administering the splice-switching ASO increases expression of DKC1 isoform 3 by at least 1.1-, 1.2-, 1.3-, 1.4-, 1.5-, 1.6-, 1.7-, 1.8-, 1.9-, 2-, 3-, 4-, 5-, or 10-fold compared to the expression of DKC1 isoform 3 in the host cell in the absence of the ASO. In some embodiments, the gsnoRNA comprises a scaffold sequence derived from a wildtype H/ACA-snoRNA selected from the group consisting of ACA19, ACA2b, ACA36, ACA44, ACA27, E2, ACA3, and ACA17. In some embodiment, the gsnoRNA comprises a scaffold sequence derived from ACA2b. In some embodiments, the gsnoRNA comprises a scaffold sequence derived from ACA36. In some embodiments, the gsnoRNA comprises a scaffold sequence derived from ACA19. In some embodiments, the gsnoRNA comprises a nucleotide sequence selected from the group consisting of SEQ ID NOs: 3-12, 15-19, 22-36, and 177-179. In some embodiments, the gsnoRNA comprises a nucleotide sequence selected from the group consisting of SEQ ID NOs: 15-19.

In some embodiments, the disease or condition is selected from the group consisting of Cystic fibrosis, Hurler Syndrome, alpha-1-antitrypsin (A1AT) deficiency, Parkinson's disease, Alzheimer's disease, albinism, Amyotrophic lateral sclerosis, Asthma, 8-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase, Haemophilia, Hereditary Hemochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer. Exemplary diseases or conditions associated with PTCs in target RNAs are listed in the Human Gene Mutation Database (HGMD®, available at hgmd.cf.ac.uk) and ClinVar database (see Landrum et al. ClinVar: improvements to accessing data. Nucleic Acids Res. 2020; 48(D1): D835-D844; available at ncbi.nlm.nih.gov/clinvar/intro). In some embodiments, threonine or serine is incorporated at the ΨAA and ΨAG codons, and phenylalanine or tyrosine at the ΨGA codons.

In some embodiments, the present disclosure provides for the use of a nucleic acid molecule (encoding an engineered gsnoRNA as described herein) in the manufacture of a medicament for the treatment of one or more of the diseases listed herein. In some embodiments, provided herein are engineered gsnoRNA for use in the treatment of cystic fibrosis (CF). Exemplary PTCs associated with CF are known in the art, for example as described in international patent publication WO2019191232, the contents of which are herein incorporated by reference in their entirety. Exemplary cystic fibrosis-associated PTC mutations include, but are not limited to, G542X (UGA), W1282X (UGA), R553X (UGA), R1162X (UGA), Y122X (UAA), W1089X, W846X, and W401X mutations, which can be modified through pseudouridylation to amino acid encoding codons, thereby allowing the translation to full length proteins. It has for instance been well established in the art that ΨAA and ΨAG codons are both translated to serine or threonine, whereas a ΨGA is translated to tyrosine or phenylalanine, instead of being seen as a stop codon (Karijolich and Yu, 2011). In some embodiments, the host cell is an archaeal or eukaryotic cell. In some embodiments, the host cell is a mammalian cell. In some embodiments, the host cell is a human cell. In some embodiments, the method is carried out in vivo. In other embodiments, the method is carried out ex vivo.

The methods of the present disclosure can be applied to suppress NMD and/or promote PTC read-through of a disease-associated PTC for a wide range of known disease-associated PTCs. There are a large number of human diseases that result from nonsense mutations in the respective disease genes. For instance, Usher syndrome is an inherited retinal dystrophy (IRD) that is the principal cause of combined deafness and blindness. Nonsense mutations occur in 12% of Usher syndrome patients and have been described in different genes, such as the USH2A gene. Some Hurler Syndrome patients, suffering from skeletal abnormalities and cognitive impairment, carry a nonsense mutation in the IDUA gene that prevents the production of a functional full-length IDUA protein in these patients. A substantial fraction of cystic fibrosis (CF) cases, a chronic disease affecting the lungs and the digestive system, is due to nonsense mutations in the CFTR gene. The PTCs resulting from these nonsense mutations are identified in the coding region at several different sites, each of which leads to total lack of functional full-length CFTR protein. Nonsense mutations are also found in some relevant oncogenes of many cancer patients, resulting in complete lack of full-length protein products. Given the deleterious role of nonsense mutations in gene expression and disease, nonsense suppression becomes an attractive strategy and the ultimate goal in combating these diseases.

### C. Delivery to Target Cells

In some aspects, the methods provided herein comprise delivering (e.g., administering) a gsnoRNA and/or DKC1 protein, or a nucleic acid encoding the gsnoRNA and/or DKC1 protein, to a host cell comprising the target RNA. The amount of nucleic acid encoding a gsnoRNA and/or DKC1 protein to be administered, the dosage and the dosing regimen can vary from cell type to cell type, the disease to be treated, the target population, the mode of administration (e.g. systemic versus local), the severity of disease and the acceptable level of side activity, but these can and should be assessed by trial and error during in vitro research, in pre-clinical and clinical trials. The trials are particularly straightforward when the modified sequence leads to an easily-detected phenotypic change.

In some embodiments, the method comprises delivering one or more nucleic acids (e.g., a gsnoRNA or a nucleic acid encoding the gsnoRNA and/or DKC1 protein) and/or a pre-formed gsnoRNA protein complex (which may comprise the gsnoRNA, DKC1 protein, NOP10 protein, GAR1 protein, and/or NHP2 protein) to a cell (e.g., a mammalian or human cell). Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, the present application further provides cells produced by such methods, and organisms (e.g., non-human mammals) comprising or produced from such cells.

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787, and 4,897,355, and lipofection reagents are sold commercially (e.g., TRANSFECTAMINE™ and LIPOFECTAMIN®). In some embodiments, LIPOFECTAMINE®2000 is used to transfect a nucleic acid encoding the gsnoRNA and/or the DKC1 protein (e.g., a nucleic acid vector encoding the gsnoRNA and/or the DKC1 protein).

One suitable trial technique involves delivering the nucleic acid molecule according to the present disclosure to cell extracts, cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained and future delivery can be performed without needing to take biopsy samples. A method of the present disclosure can thus include a step of identifying the presence of the desired change in the cell's target RNA sequence, thereby verifying that the target RNA sequence has been modified. The change may be assessed on the level of the protein (length, glycosylation, function or the like), or by some functional read-out, such as a(n) (inducible) current, when the protein encoded by the target RNA sequence is an ion channel, for example. In the case of CFTR function, an Ussing chamber assay or an NPD test in a mammal, including humans, are well known to a person skilled in the art to assess restoration or gain of function.

After pseudouridylation has occurred in a cell, the modified RNA can become diluted over time, for example due to cell division, limited half-life of the edited RNAs, etc. Thus, in practical therapeutic terms a method of the present disclosure may involve repeated delivery of an oligonucleotide until enough target RNAs have been modified to provide a tangible benefit to the patient and/or to maintain the benefits over time.

In some embodiments, gsnoRNAs can be delivered to cells in the form of a naked nucleic acid. One other way by which such constructs (a gsnoRNA and/or DKC1 protein, or a nucleic acid encoding the gsnoRNA and/or DKC1 protein) can be delivered to the cell (either in vitro, ex vivo or in vivo) is by using a delivery vehicle such as a viral vector.

Conventional viral based systems for nucleic acid delivery include retroviral, lenti virus, adenoviral, adeno-associated and herpes simplex virus vectors. Integration in the host genome is possible with the retrovirus, lentivirus, and adeno-associated virus methods, often resulting in long term expression of the inserted transgene. Additionally, high transduction efficiencies have been observed in many different cell types. The tropism of a retrovirus can be altered by incorporating foreign envelope proteins, expanding the potential target population of target cells. Lentiviral vectors are retroviral vectors that are able to transduce or infect non-dividing cells and typically produce high viral titers. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the nucleic acids into the target cell to provide permanent transgene expression. Widely used retroviral vectors include those based upon murine leukemia vims (MuLV), gibbon ape leukemia virus (GaLV), Simian Immuno deficiency vims (SIV), human immuno deficiency vims (HIV), and combinations thereof. In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system.

Packaging cells are typically used to form virus particles that are capable of infecting a host cell. Such cells include 293T cells, which package adenovirus, and ψ2 cells or PA317 cells, which package retrovirus. Viral vectors are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, the viral vector is based on Adeno-Associated Virus (AAV). In some embodiments, the viral vector is for instance a retroviral vector such as a lentivirus vector and the like. Also, plasmids, artificial chromosomes, and plasmids usable for targeted homologous recombination and integration in the human genome of cells may be suitably applied for delivery of a gsnoRNA as described herein. In some embodiments, when the gsnoRNA is delivered by a viral vector, it is in the form of an RNA transcript that comprises the sequence of an oligonucleotide according to the present disclosure in a part of the transcript. In some embodiments, an AAV vector according to the present disclosure is a recombinant AAV vector and refers to an AAV vector comprising part of an AAV genome comprising an exon-intron-exon sequence according to the present disclosure encapsidated in a protein shell of capsid protein derived from an AAV serotype. Part of an AAV genome may contain the inverted terminal repeats (ITR) derived from an adeno-associated virus serotype, such as AAV1, AAV2, AAV3, AAV4, AAV5, AAV6, AAV7, AAV8, AAV9 and others. Protein shell comprised of capsid protein may be derived from an AAV serotype such as AAV1, 2, 3, 4, 5, 6, 7, 8, 9 and others. A protein shell may also be named a capsid protein shell. AAV vector may have one or all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the present disclosure a capsid protein shell may be of a different serotype than the AAV vector genome ITR. An AAV vector according to the present disclosure may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV2 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the present disclosure. In some embodiments, a recombinant AAV vector according to the present disclosure comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

In some embodiments, a recombinant AAV vector according to the present disclosure comprises a capsid protein shell of AAV serotype 2 and the AAV genome or ITRs present in said vector are derived from AAV serotype 5; such vector is referred to as an AAV 2/5 vector. In some embodiments, a recombinant AAV vector according to the present disclosure comprises a capsid protein shell of AAV serotype 2 and the AAV genome or ITRs present in said vector are derived from AAV serotype 8; such vector is referred to as an AAV 2/8 vector. In some embodiments, a recombinant AAV vector according to the present disclosure comprises a capsid protein shell of AAV serotype 2 and the AAV genome or ITRs present in said vector are derived from AAV serotype 9; such vector is referred to as an AAV 2/9 vector. In some embodiments, a recombinant AAV vector according to the present disclosure comprises a capsid protein shell of AAV serotype 2 and the AAV genome or ITRs present in said vector are derived from AAV serotype 2; such vector is referred to as an AAV 2/2 vector. In some embodiments, a nucleic acid molecule harboring an exon-intron-guide RNA-intron-exon sequence according to the present disclosure represented by a nucleic acid sequence of choice is inserted between the AAV genome or ITR sequences as identified above, for example an expression construct comprising an expression regulatory element operably linked to a coding sequence and a 3′ termination sequence. “AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art. The AAV helper functions can be supplied on an AAV helper construct, which may be a plasmid.

Introduction of the helper construct into the host cell can occur e.g. by transformation, transfection, or transduction prior to or concurrently with the introduction of the AAV genome present in the AAV vector as identified herein. The AAV helper constructs of the present disclosure may thus be chosen such that they produce the desired combination of serotypes for the AAV vector's capsid protein shell on the one hand and for the AAV genome present in said AAV vector replication and packaging on the other hand. “AAV helper virus” provides additional functions required for AAV replication and packaging.

Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456. In some embodiments, an AAV genome as present in a recombinant AAV vector according to the present disclosure does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art. In some embodiments, an AAV vector according to the present disclosure is an AAV2/5, AAV2/8, AAV2/9 or AAV2/2 vector.

In some embodiments, the gsnoRNA and DKC1 are delivered to the cell as a ribonucleoprotein complex (e.g., a complex comprising the gsnoRNA. DKC1, NOP10, GAR1, and/or NHP2). Methods for intracellular delivery of protein or protein complexes, such as pre-formed gsnoRNA-DKC1/NOP10/GAR1/NHP2 complex, include, but are not limited to, mechanical methods, such as microinjection, electroporation and mechanical deformation of cells using a microfluidic device; carrier-based methods, such as cell-penetrating peptides (CPPs), virus-like particles, supercharged proteins, nanocarriers, supramolecular carrier-based delivery systems, and nanoparticle-stabilized nanocapsules. See, for example, Fu et al. Bioconjugate Chem. 2014, 25, 1602-1608. Some mechanical methods, such as microinjection and electroporation, can be invasive, and low-throughput. In some embodiments, the ribonucleoprotein complex is delivered into the cell by inserting the complex through the cell membrane while passing cells through a microfluidic system, such as CELL SQUEEZE® (see, for example, U.S. Patent Application Publication No. 20140287509).

As described above, introduction of the nucleic acid molecule according to the present disclosure into the cell is performed by general methods known to the person skilled in the art. After pseudouridylation, the read-out of the effect (alteration of the target RNA sequence) can be monitored through different ways in an optional identification step. Hence, the identification step of whether the desired pseudouridylation of the target uridine has indeed taken place depends generally on the position of the target uridine in the target RNA sequence, and the effect that is incurred by the presence of the uridine (point mutation, PTC). Hence, in some embodiments, depending on the ultimate effect of U to Ψ conversion, the identification step comprises: assessing the presence of a functional, elongated, full length and/or wild type protein; assessing whether splicing of the pre-mRNA was altered by the pseudouridylation; or using a functional read-out, wherein the target RNA after the pseudouridylation encodes a functional, full length, elongated and/or wild type protein. The functional assessment for each of the diseases mentioned herein will generally be according to methods known to the skilled person.

The nucleic acid molecule, such as a gsnoRNA expression construct or vector according to the present disclosure is suitably administrated in aqueous solution, e.g. saline, or in suspension, optionally comprising additives, excipients and other ingredients, compatible with pharmaceutical use. Administration may be by inhalation (e.g. through nebulization), intranasally, orally, by injection or infusion, intravenously, subcutaneously, intra-dermally, intra-cranially, intravitreally, intramuscularly, intra-tracheally, intra-peritoneally, intra-rectally, and the like. Administration may be in solid form, in the form of a powder, a pill, or in any other form compatible with pharmaceutical use in humans. The present disclosure is particularly suitable for treating genetic diseases, such as CF.

In some embodiments the nucleic acid molecule, such as a gsnoRNA, expression construct or vector can be delivered systemically. In some embodiments, the nucleic acid molecule, such as a gsnoRNA, expression construct or vector can be delivered to cells or delivered locally to a tissue in which the target sequence's phenotype is seen. For instance, mutations in CFTR cause CF which is primarily seen in lung epithelial tissue, so with a CFTR target sequence in some embodiments the deliver the oligonucleotide construct specifically and directly to the lungs. This can be conveniently achieved by inhalation e.g. of a powder or aerosol, typically via the use of a nebuliser. In some embodiments, the nebulizer is a nebulizer that uses a so-called vibrating mesh, including the PARI eFlow (Rapid) or the i-neb from Respironics. It is to be expected that inhaled delivery of oligonucleotide constructs according to the present disclosure can also target these cells efficiently, which in the case of CFTR gene targeting could lead to amelioration of gastrointestinal symptoms also associated with CF. In some diseases the mucus layer shows an increased thickness, leading to a decreased absorption of medicines via the lung. One such a disease is chronical bronchitis, another example is CF. A variety of mucus normalizers are available, such as DNases, hypertonic saline or mannitol, which is commercially available under the name of Bronchitol. When mucus normalizers are used in combination with pseudouridylating oligonucleotide constructs, such as the gsnoRNA constructs according to the present disclosure, they might increase the effectiveness of those medicines. Accordingly, administration of an oligonucleotide construct according to the present disclosure to a subject, such as a human subject, may be combined with mucus normalizers. In addition, administration of the oligonucleotide constructs according to the present disclosure can be combined with administration of small molecule for treatment of CF, such as potentiator compounds for example Kalydeco (ivacaftor; VX-770), or corrector compounds, for example VX-809 (lumacaftor) and/or VX-661. Alternatively, or in combination with the mucus normalizers, delivery in mucus penetrating particles or nanoparticles can be applied for efficient delivery of pseudouridylating molecules to epithelial cells of for example lung and intestine. In some embodiments, administration of an oligonucleotide construct according to the present disclosure to a subject, such as a human subject, is combined with antibiotic treatment to reduce bacterial infections and the symptoms of those such as mucus thickening and/or biofilm formation. The antibiotics can be administered systemically or locally or both. For application in CF patients the oligonucleotide constructs according to the present disclosure, or packaged or complexed oligonucleotide constructs according to the present disclosure may be combined with any mucus normalizer such as a DNase, mannitol, hypertonic saline and/or antibiotics and/or a small molecule for treatment of CF, such as potentiator compounds for example ivacaftor, or corrector compounds, for example lumacaftor and/or VX-661. To increase access to the target cells, Broncheo-Alveolar Favage (BAF) could be applied to clean the lungs before administration of the oligonucleotide according to the present disclosure.

### IV. Pharmaceutical Compositions, Kits, and Articles of Manufacture

In some aspects, provided herein is a pharmaceutical composition comprising any of the gsnoRNAs, nucleic acid constructs/molecules, or engineered RNA-editing systems described herein, and a pharmaceutically acceptable carrier.

Pharmaceutical compositions can be prepared by mixing the therapeutic agents described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

In some embodiments, the pharmaceutical composition comprises a gsnoRNA. In other embodiments, the pharmaceutical composition comprises a nucleic acid construct (e.g., a vector such as a plasmid or viral vector) encoding the gsnoRNA. In some embodiments, the pharmaceutical composition comprises free gsnoRNAs (‘naked’ gsnoRNAs), or gsnoRNAs conjugated to other components, such as ligands for targeting, for uptake and/or for intracellular trafficking. gsnoRNAs may be used in aqueous solutions (generally pharmaceutically acceptable carriers and/or solvents), or formulated using transfection agents, liposomes or nanoparticulate forms (e.g. SNALPs, LNPs and the like). Such formulations may comprise functional ligands to enhance bioavailability and the like.

The present application further provides kits and articles of manufacture for use in any embodiment of the treatment methods described herein. The kits and articles of manufacture may comprise any one of the formulations and pharmaceutical compositions described herein.

In some aspects, provided herein is a kit for editing a target RNA in a host cell, comprising any of the gsnoRNA or nucleic acid molecules described in Section II B. In some embodiments, the kit further comprises an agent for enhancing expression of an endogenous DKC1 isoform 3 in the host cell. In some embodiments, the kit comprises a splice-switching antisense oligonucleotide (ASO), wherein the ASO enhances expression of a DKC1 protein that is an endogenous DKC1 isoform with cytoplasmic localization in the host cell. In some embodiments, the kit further comprises a DKC1 protein or nucleic acid encoding a DKC1 protein. In some embodiments, the DKC1 protein is a DKC1 isoform (e.g., isoform 3) with cytoplasmic localization. In some embodiments, the DKC1 protein is part of a ribonucleoprotein (RNP) complex that associates with the gsnoRNA. In some embodiments, the DKC1 protein comprises a deletion of a nuclear localization signal (NLS) relative to a wildtype DKC1 protein of the same species. In some embodiments, the DKC1 protein is a truncated DKC1 variant or a DKC1 variant comprising a deletion, such as any of the truncation or deletion variants described in Section II A above. In some embodiments, the kit further includes instructions for editing the target RNA according to any of the methods described herein.

In some aspects, provided herein is a kit for editing a target RNA in a host cell, comprising an engineered RNA-editing system, wherein the engineered RNA editing system comprises: (a) a gsnoRNA comprising a guide sequence that hybridizes to a sequence comprising a target uridine residue in a target RNA in a host cell, or a nucleic acid molecule encoding the gsnoRNA; and (b) a DKC1 protein, or a nucleic acid molecule encoding the DKC1 protein, wherein the gsnoRNA is capable of recruiting the DKC1 protein to modify the target uridine residue into a pseudouridine residue in the target RNA. In some embodiments, the DKC1 protein is a DKC1 isoform with cytoplasmic localization. In some embodiments, the DKC1 protein is a DKC1 isoform (e.g., isoform 3) with cytoplasmic localization. In some embodiments, the DKC1 protein is part of a ribonucleoprotein (RNP) complex that associates with the gsnoRNA. In some embodiments, the DKC1 protein comprises a deletion of a nuclear localization signal (NLS) relative to a wildtype DKC1 protein of the same species. In some embodiments, the DKC1 protein is a truncated DKC1 variant or a DKC1 variant comprising a deletion, such as any of the truncation or deletion variants described in Section II A above. In some embodiments, the kit further includes instructions for editing the target RNA according to any of the methods described herein.

The kits of the present disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the gsnoRNA and/or DKC1 protein, or nucleic acid molecules encoding the gsnoRNA and/or DKC1 protein as disclosed herein to provide effective treatment of an individual or of many individuals. Additionally, kits may be provided that contain sufficient dosages of the gsnoRNA and/or DKC1 protein, or nucleic acid molecules encoding the gsnoRNA and/or DKC1 protein to allow for multiple administrations to an individual. Kits may also include multiple unit doses of the pharmaceutical compositions and instructions for use and packaged in quantities sufficient for storage and use in pharmacies, for example, hospital pharmacies and compounding pharmacies.

In some embodiments, the kit comprises a delivery system. The delivery system may be a unit dose delivery system. Delivery systems for these various dosage forms can be syringes, dropper bottles, plastic squeeze units, atomizers, nebulizers or pharmaceutical aerosols in either unit dose or multiple dose packages. In some embodiments, there is provided a delivery system of any one of the gsnoRNA and/or DKC1 protein, or nucleic acid molecules encoding the gsnoRNA and/or DKC1 protein described herein, comprising the gsnoRNA and/or DKC1 protein, or nucleic acid molecules encoding the gsnoRNA and/or DKC1 protein and a device for delivering the gsnoRNA and/or DKC1 protein, or nucleic acid molecules encoding the gsnoRNA and/or DKC1 protein.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### EXAMPLES

The present disclosure will be more fully understood by reference to the following examples. They should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1. Exploiting Engineered Guide snoRNA for Readthrough of Premature Termination Codon

This example demonstrates the efficiency of pseudouridylation of target RNAs (e.g., mRNA pseudouridylation evidenced by pseudouridylation-dependent PTC-read-through) by engineered guide snoRNAs, and provides different expression systems for guide snoRNAs.

To achieve site-specific pseudouridylation in mRNA in vivo, artificial guide snoRNAs (gsnoRNAs) were engineered to target specific mRNAs for modification (FIG. 1A). H/ACA snoRNAs contain two hairpins followed by the H and ACA box motifs, and both hairpins of the engineered snoRNAs provided herein contained the guide sequences that are capable of targeting the PTC site. To assess the efficiency of PTC-read-through, a Venus reporter (Reporter-1) was designed, which expresses the Venus fluorescent reporter gene with an amber codon (TAG) inserted between the 154^{th} and 155^{th} amino acid codons, to prematurely terminate the Venus translation. Such a reporter allows measurement of the efficiency of PTC-read-through by monitoring the expression level of Venus. A positive control with a glycine codon (GGT) into the same position was included (FIG. 1B). The Reporter-1 (Venus-TAG) or control (Venus-GGT) was co-transfected with gsnoRNA expression constructs (gCtrl (SEQ ID NO: 14)gACA19 (SEQ ID NO: 37), gACA44 (SEQ ID NO: 38), gACA27 (SEQ ID NO: 39), gE2 (SEQ ID NO: 40), gACA19-S (SEQ ID NO: 41), or gACA19-L (SEQ ID NO: 42) into HEK293T cells in order to assay PTC-read-through as a result of modifying the corresponding stop codon with snoRNA-guided pseudouridylation. The effects of PTC-read-through were measured by a high content imaging system and quantified by comparing with the positive control group (FIGS. 1C, 1D). The relative Venus expression is reported as the percent (%) of Venus detected compared to control (Venus-GGT). These gsnoRNAs served as the first-generation RESTART (RESTART v1).

The sequence of the control gsnoRNA (gCtrl) is provided below, with guide regions underlined (SEQ ID NO: 14):

CAGCAAGCAUCGAGGGGCUGUGGCUGGUCAUAGCCAUGGGAUCGUACUCC
GCAUGCAAGAGCAACCUGGAAAGACAGUGACAGCGCAGGUCAGUACAAUA
CCUGCAAGCUGCAUGCCAGCUUUCCUAUAAUG

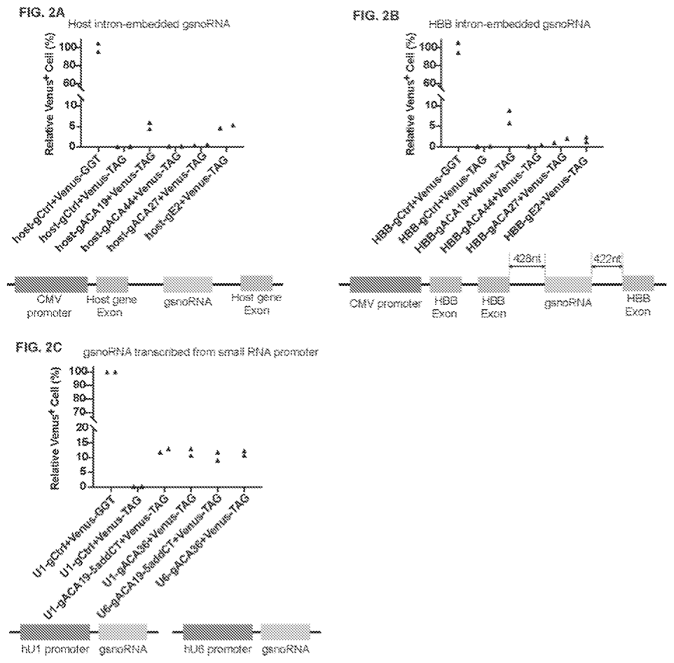

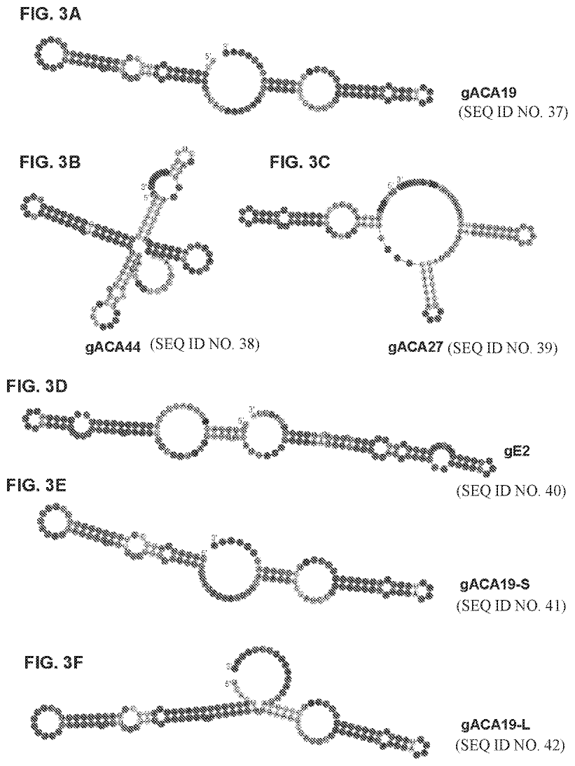

In the human genome, more than 90% of snoRNA genes are encoded in pre-mRNA introns¹. The present inventors first evaluated the effect of PTC-read-through mediated by several gsnoRNAs located in host gene introns (RESTART v1.0). The present inventors first selected 4 endogenous snoRNAs that have high expression levels in human², including ACA19, ACA44, ACA27, and E2 (within EIF3A, SNHG12, RPL21, RPSA host genes, respectively) (FIGS. 2A-2C and FIGS. 3A-3F), and engineered gsnoRNAs based on these scaffolds to target the Venus reporter PTC. The host gene fragments comprising the snoRNAs were cloned into a construct driven by a CMV promoter. After co-transfecting the Reporter-1 with these gsnoRNA expressing constructs, (host-gCtrl; host-gACA19, host gACA-44, host-gACA27, and host-gE2) evidence of PTC-read-through indicated by the Venus expression was observed: 5.2% and 5.0% Venus positive cells (compared to the control Venus-GGT reporter) were detected from cells transfected with host-gACA19 and host-gE2, respectively, while others displayed negligible signals (FIG. 2A). The Venus expression was clearly sequence-dependent because the control gsnoRNA (gCtrl) could not activate Venus expression. The present inventors realized that the gACA19 and gE2 scaffolds, which displayed higher activity that the other scaffolds, were predicted to have more stable secondary structures than gACA44 and gACA27 (FIGS. 3A-3D), suggesting that the higher efficiency of target modification evidenced by the PTC read-through may be correlated with stability of secondary structures. To test the effects of gsnoRNA-carrying host genes sequences on PTC-read-through efficiencies, the present inventors carried out further comparison by cloning the different gsnoRNAs into the intron between Exon2 and Exon3 of Hemoglobin subunit β (HBB) gene. The gACA19 again showed the highest efficiency (relative Venus positive cells: 7.3%) in mediating PTC-read-through for Reporter-1, and gE2 showed the second highest efficiency (relative Venus positive cells: 1.8%) (FIG. 2B).

Based on the present inventors' observation that host gene sequences have divergent effects on different gsnoRNAs (as shown in FIGS. 2A-2B), the present inventors envisioned that directly expressing the gsnoRNAs without host gene effects might further increase the efficiency of PTC-read-through. Therefore, the present inventors designed a series of gsnoRNA expression constructs driven by hU6 (type III RNA polymerase III promoter) and hU1 (snRNA-type RNA polymerase II promoter) promoters (RESTART v1.1) (FIGS. 1C-1D and FIG. 2C), and co-transfected them together with Reporter-1 into HEK293T cells. The PTC-read-through efficiency of hU6 promoter-driven gACA19 increased for 1.9- and 1.3-fold compared to that of host gene intron- and HBB intron-embedded gACA19, respectively (FIGS. 1C-1D and FIGS. 2A-2B). The efficiency of hU6 promoter-driven gsnoRNAs are similar to that of hU1 promoter-driven gsnoRNAs (FIG. 2C). The effects of PTC-read-through were further characterized by extending or truncating gsnoRNA: no obvious effects were observed from extending the gACA19 (gACA19-L, 9 nt on 5′ and 9 nt on 3′) while the shortened gACA19 (gACA19-S, 3 nt on 5′) reduced the Reporter-1 PTC read-through efficiency to 35% compared to full-length gACA19 (FIG. 1D and FIGS. 2E-2F). Since gsnoRNAs driven by a small RNA promoter displayed higher efficiency in mediating PTC-read-through compared to intron-embedded gsnoRNAs, the present inventors selected gsnoRNAs driven by hU6 promoter to conduct subsequent analysis.

To determine whether endogenous DKC1 proteins are responsible for the above observation, the present inventors carried out RESTART v1. 1 on DKC1 stably knockdown (DKC1 KD) HEK293T cells (FIG. 1E). No PTC-read-through was observed for gsnoRNAs from DKC1-KD cells, while these gsnoRNAs activated the expression of Venus in control groups (FIG. 1F), supporting the role of endogenous DKC1 in mediating PTC-read-through of Reporter-1 (FIG. 1A). Collectively, these observations demonstrated that the gsnoRNAs can induce the PTC-read-through of targeted transcripts.

### Example 2: Optimization of gsnoRNA Scaffolds Improves the Efficiency of PTC-Read-Through

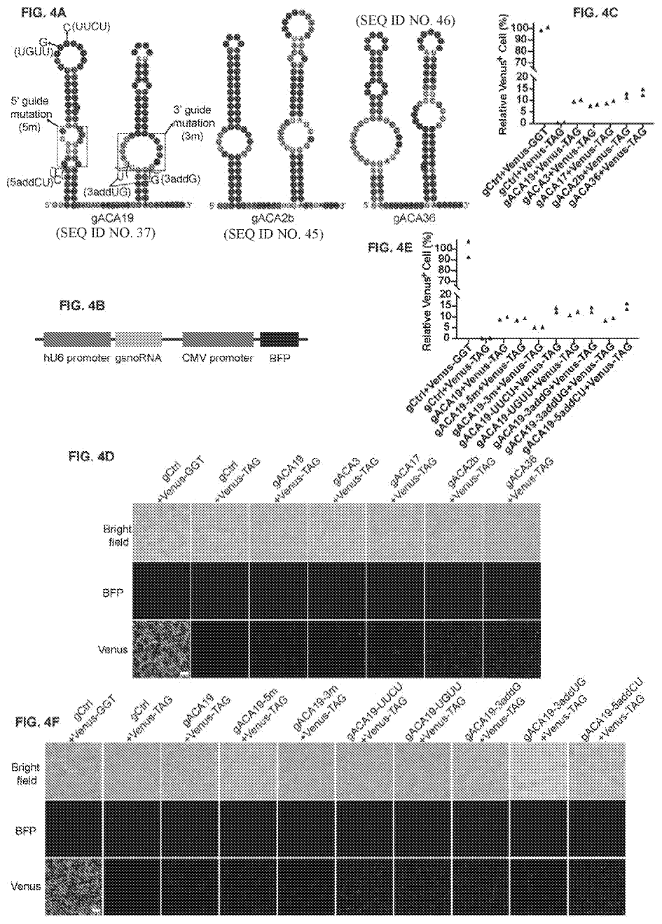

To identify optimal gsnoRNA scaffolds, the present inventors selected five snoRNAs (gACA3, gACA17, gACA19, gACA2b, and gACA36) with stable secondary structures predicted by RNAfold³ as candidate scaffolds for further characterization (RESTART v1.2) (FIG. 4A and FIG. 5A). The present inventors designed a snoRNA expression construct consisting of hU6 promoter-driven gsnoRNA and CMV promoter-driven BFP gene which was utilized to normalize the transfection efficiency (FIG. 4B). Among them, gACA36 and gACA2b outcompeted gACA19, and displayed the highest efficiencies of PTC-read-through (relative Venus positive cells: 13.7% and 12.2%, respectively) (FIGS. 4C-4D). gACA19 has a minimum free energy of −37.10 kcal/mol, gACA2b has a minimum free energy of —54.90 kcal/mol, and gACA36 has a minimum free energy of —43.50 kcal/mol. There does not seem to be a direct relationship between the stability of gsnoRNA scaffolds and editing efficiency.

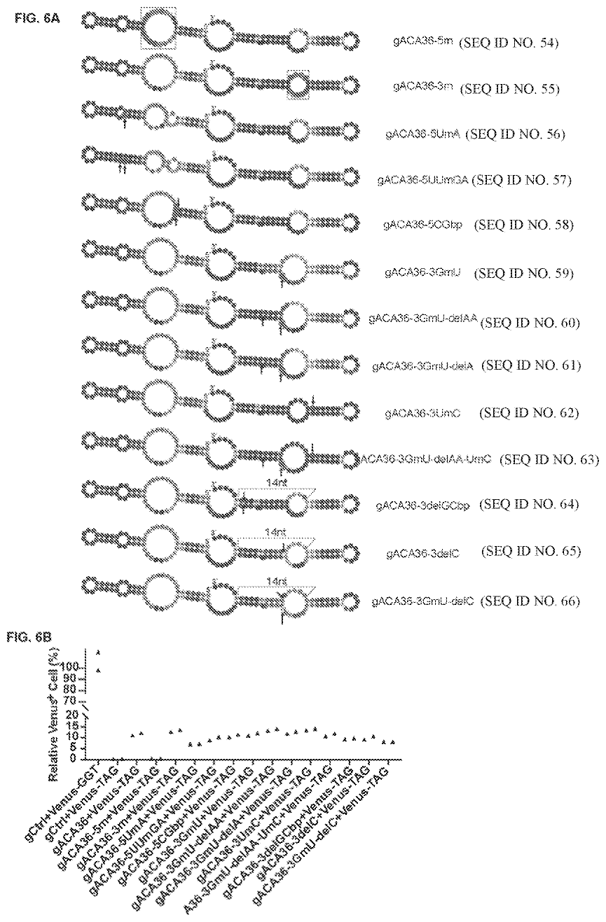

To investigate the roles of the two hairpins of gsnoRNAs, the present inventors introduced mutations in 5′ and 3′ guide elements, respectively (FIG. 4A, FIG. 5A, and FIG. 6A). The editing efficiency of gACA19 5′ hairpin mutation (gACA19-5m) was comparable to that of gACA19, while gACA19-3m displayed reduced efficiency (FIGS. 4E-4F). For gACA36, the editing efficiency of gACA36-3m was comparable to that of gACA36, while gACA36-5m displayed negligible signals (FIG. 6B). These results indicated that only one hairpin of gACA19/gACA36 plays a leading role, and the two hairpins of gsnoRNA targeting the same site might not compete with each other.

The present inventors next sought to further improve the PTC read-through efficiency by engineering the gsnoRNA scaffolds (RESTART v1.3) (FIG. 4E-4F and FIGS. 3-4). Given that RNA polymerase III terminates transcription at small polyUs stretch, the present inventors introduced a single base mutation to “UUUU” sequence in the apical loop of gACA19 (FIG. 4A and FIG. 5C). Notably, both gACA19-UUCU and gACA19-UGUU showed improvements (FIGS. 4E-4F). Without being bound by theory, the present inventors realized that altering the distance in the gsnoRNA hairpin so that the distance between the nucleotide in the guide region that hybridizes to the target uridine and the H/ACA box is 14 nucleotides increased the editing efficiency of the gsnoRNA. In an example, the present inventors inserted a single base after U115 of gACA19, so that the distance between the nucleotide in the guide region that hybridizes to the target uridine and the H/ACA box is 14 nucleotides (FIG. 4A and FIG. 5D): gACA19-3addG increased the efficiencies to 1.4-fold compared to unmodified gACA19 (FIGS. 4E-4F). Furthermore, without being bound by theory, the present inventors discovered that making the guide elements of the gsnoRNA hairpin more open (e.g., decreasing the base-pairing probability of the secondary structure within the guide region) could increase the editing efficiency of a gsnoRNA. To make the guide elements more open, the present inventors inserted dinucleotide after U8 in the 5′ hairpin of gACA19 (FIG. 4A and FIG. 5D). Notably, gACA19-5addCU increased the PTC-read-through efficiencies for 60% (FIGS. 4E-4F). However, engineered gACA36 scaffolds did not further improve the efficiency of PTC-readthrough (FIGS. 6A-6B). The present inventors also combined optimized mutations of gACA19 and expressed two tandem gsnoRNAs, but they did not further improve the efficiency either.

### Example 3: The Spatial Proximity Effect of gsnoRNA and Target PTC Site

The present inventors next asked if spatial proximities of gsnoRNA and target PTC site have impacts on the efficiency of PTC-readthrough. The inventors designed two new reporters: (1) the Report-2 contains a PTC site in between mCherry and EGFP coding regions, and activated by gsnoRNAs from RESTART v1.3. mCherry was utilized to normalize the transfection efficiency. (2) in Reporter-3 (RESTART v1.4), the gsnoRNA is arranged in tandem with the PTC reporter, which is the same PTC reporter as Reporter-2. The gsnoRNAs have comparable efficiencies in suppressing PTC of both Reporter-2 and Reporter-1, indicating gsnoRNAs work for different reporters. Unexpectedly, gsnoRNAs had increased PTC-read-through efficiencies in Reporter-3 (relative EGFP positive cells: ˜30%, ˜2-fold compared to RESTART v1.3).

### Example 4. RESTART Enables PTC-Read-Through in Multiple Cell Lines

The present inventors tested RESTART v1.4 in four different cell lines that originated from distinct tissues, including three human cell lines and one murine cell line. Efficient PTC-read-through events were observed for all cell lines tested, suggesting that the gsnoRNA design of the present disclosure is a versatile strategy to suppress PTC in different mammalian cell types.

### Example 5. Increasing DKC1-Isoform3 Expression Significantly Improves PTC-Read-Through

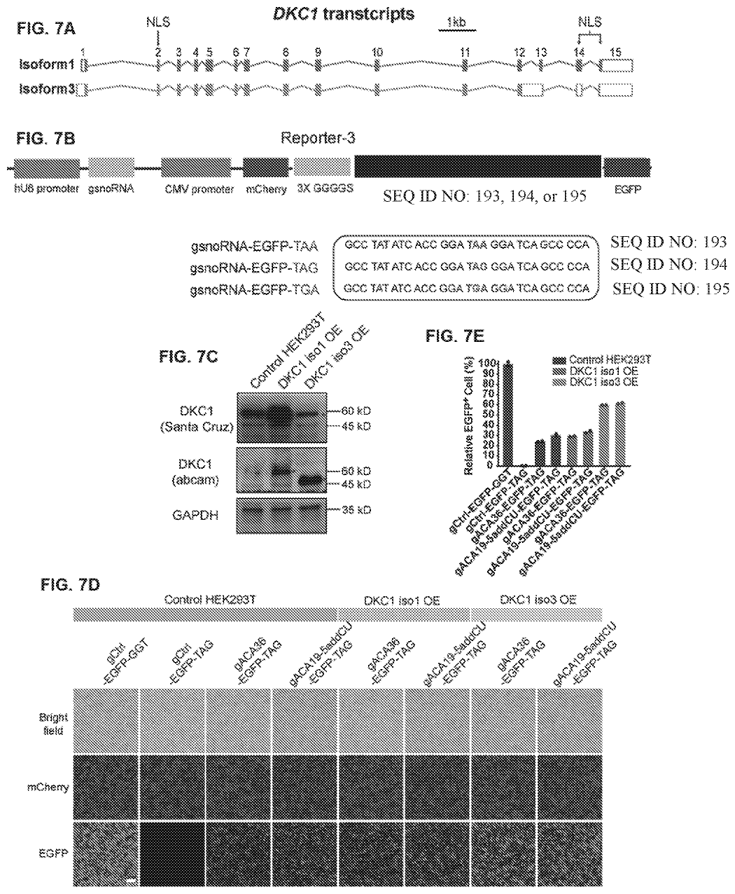

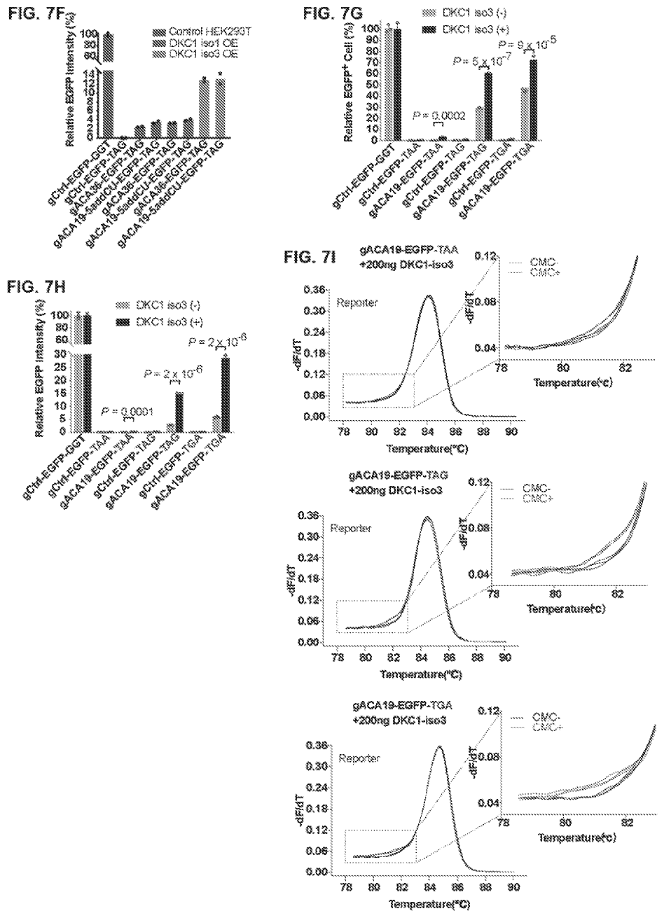

Notably, neither combining of optimized mutations nor increasing the gsnoRNA expression level by transfecting construct of two tandem gsnoRNAs further increased the PTC-read-through, suggesting RESTART v1.3 offers gsnoRNAs with optimal structure and expression levels. Based on the present inventor's realization that the engineered gsnoRNAs of the present disclosure provided optimized gsnoRNA structure and expression levels, the present inventors wondered whether enzyme levels and accessibility, rather than gsnoRNA stability and expression, might be rate-limiting factors. DKC1 is responsible for snoRNA-guided deposition of pseudouridine and the accompanied PTC read-through in RESTART (FIGS. 1A, 1F). There are two DKC1 isoforms in human cells: DKC1 isoform1 is the canonical DKC1 form containing the bipartite N- and C-terminal nuclear localization signals (NLSs); DKC1 isoform3 is an alternatively splicing variant, which is produced by retention of the intron 12 and lacks C-terminal NLS (FIG. 7A). The endogenous mRNA expression level of isoform 1 is approximately 20-fold greater than that of isoform3⁴.

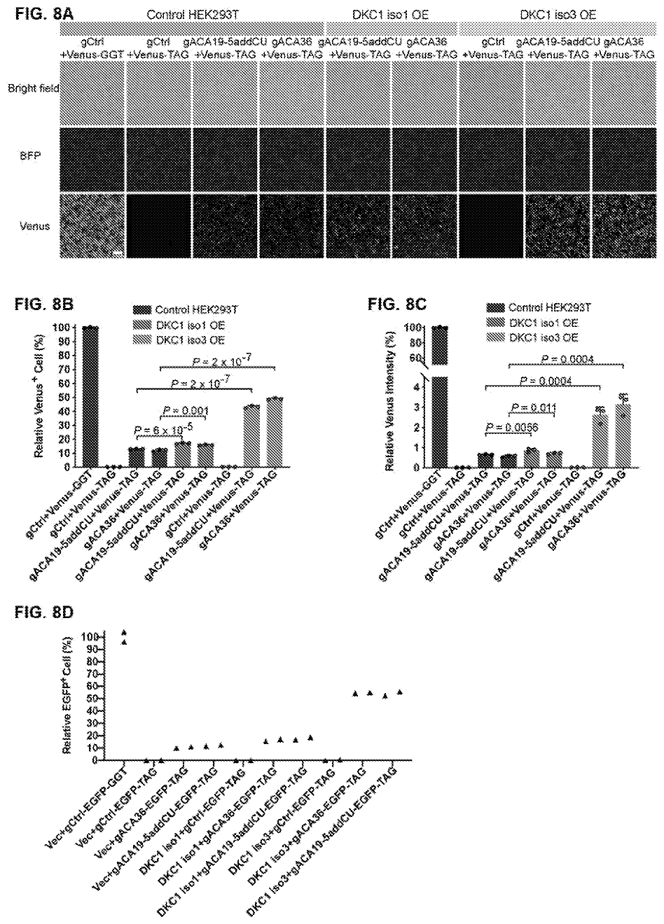

First, the present inventors generated DKC1 stable overexpressing cell lines, and transfected said DKC1-isoform1 overexpressing cells with Reporter-3 (FIGS. 7B-7C). DKC1-isoform1 overexpression only slightly increased the relative fraction of EGFP positive cells and the relative EGFP intensities to 1.2- and 1.3-fold compared to that of control cells, respectively (FIGS. 7D-7F). Surprisingly, in isoform3 overexpressed cells, the relative fraction of EGFP positive cells and relative EGFP intensities were greatly increased to 2.5- and 5.2-fold, respectively (FIGS. 7D-7F). These observations were further confirmed by co-transfecting Reporter-1 and gsnoRNA constructs into DKC1 stable overexpressing cells (FIGS. 8A-8C). To further investigate DKC1 transient roles, Reporter-3 was co-transfected together with DKC1 expressing constructs. Again, isoform3 transient overexpressing greatly increased the PTC-readthrough (FIG. 8D). We also deleted the N-terminal NLS of DKC1-isoform3, and these truncations had similar efficiency of PTC-read-through as isoform3 (FIG. 9). These unexpected results demonstrate that exogenous DKC1-isoform3 can significantly improve the efficiency of PTC-read-through, achieving 61.4% EGFP positive cells (relative to control reporter) and 13.2% EGFP intensities (relative to control reporter). The gsnoRNAs and DKC1-isoform 3 served as the second-generation RESTART (RESTART v2).

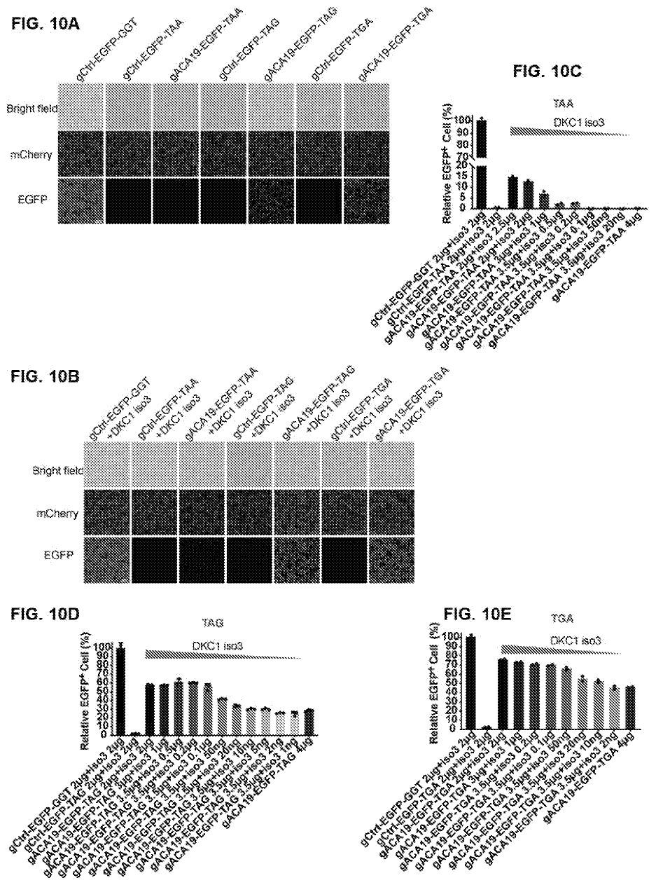

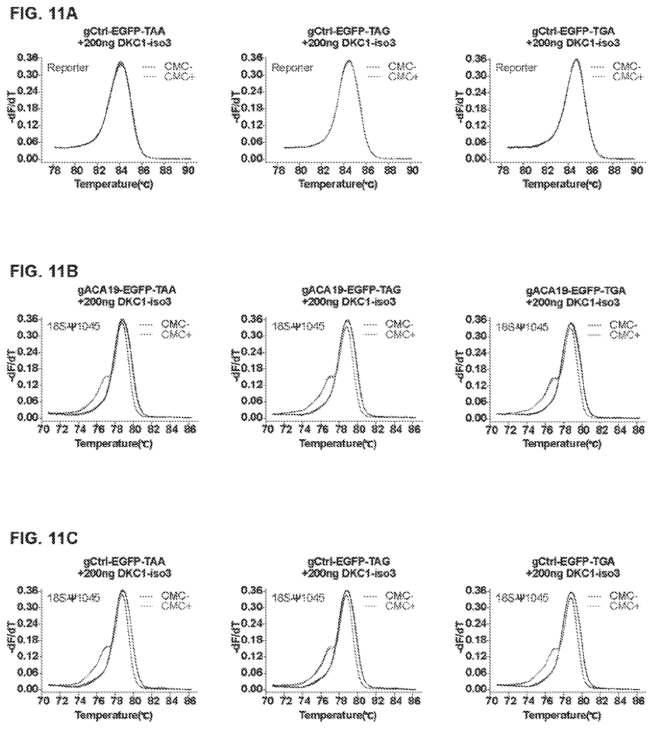

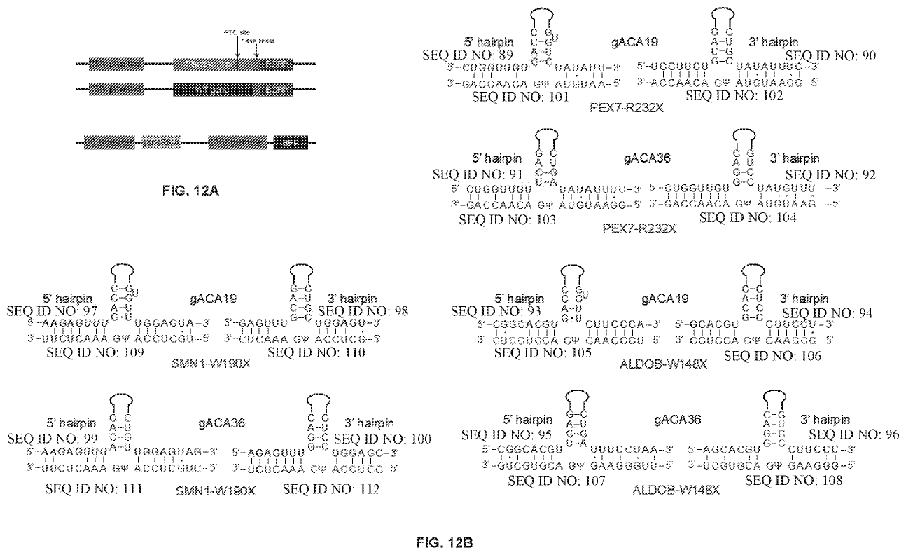

To better characterize RESTART, an additional set of Reporter-3s was constructed to include all three types of stop codons, and the resulting reporter constructs were transfected into HEK293T with and without exogenous DKC1-isoform3 (FIG. 7B). The efficiency of RESTART-mediated read-through correlated positively with that of basal or drug-induced translational readthrough⁵, with the highest read-through at opal codon (UGA), followed by amber codon (UAG) and then ochre codon (UAA) (FIGS. 7G-7H, and FIGS. 10A-10D). For the UGA (opal) codon, the relative fraction of EGFP positive cells and relative EGFP intensities were 45.3% and 5.8% (RESTART v1.4), and 72.3% and 28.6% (RESTART v2), respectively; while UAA codon displayed negligible signals without exogenous DKC1 (RESTART v1.4), and relative 2.9% EGFP positive cells and relative 0.2% EGFP intensities with DKC1-isoform3 overexpressing (RESTART v2) (FIGS. 7G-7H, and FIGS. 10A-10B). Increasing the amount of DKC1-isoform3 expressing constructs improved the PTC-readthrough of UAA (ochre) codon to relative 14.8% EGFP positive cells, while still 25% and 19% compared to UAG (amber) and UGA (opal), respectively (FIGS. 10C-10E). Together, RESTART promoted read-through of all three nonsense codons.

Next, Reporter-3 constructs of each of the three stop codons were individually co-transfected together with 200 ng DKC1-isoform3 expression construct into HEK293T cells. The locus-specific pseudouridine modification of the target was detected by a radiolabeling-free, qPCR-based method⁶ (FIG. 7I and FIGS. 11A-11C). Alterations to the melting curves were observed for all three stop codons (FIG. 7I), while negligible alteration was observed for the gCtrl group that was devoid of Ψ modification (FIG. 11A). In contrast, melting-curve alterations for Ψ1045 sites in 18S rRNA were comparable between gACA19 and gCtrl groups (FIGS. 11A-11C). Collectively, these results demonstrate that gsnoRNA-guided pseudouridylation by DKC1-isoform3 can efficiently facilitate the read-through of all three PTC codons.

### Example 6. RESTART Suppresses Disease-Relevant PTCs

This Example demonstrates correction of disease-relevant premature termination codons (PTCs) using RESTART. RNA-guided pseudouridylation of disease-relevant PTCs by the RESTART system resulted in expression of full-length gene products. Furthermore, restoration of protein function using RESTART was demonstrated for a CFTR gene containing a disease-relevant PTC. In the following example, “X” indicates a stop codon mutation. Sequences of the gsnoRNAs tested are provided in Table 4.

PTC-disease reporters were constructed in which a disease gene containing the PTC site was followed by EGFP (as shown in FIG. 12A). gACA19- and gACA36-based gsnoRNAs were designed and tested targeting seven disease-relevant nonsense mutations from six pathogenic genes, PEX7, SMN1, ALDOB, C8orf37, PCCB, and CBS (FIGS. 12B-13). By co-expressing gsnoRNA/PTC-disease gene pairs in HEK293T cells (RESTARTv1), PTC-readthrough was achieved at all sites: 6.7% (cells expressing ALDOB-W148X), 25.2% (SMN1-W190X), 33.8% (PEX7-R232X), 1.7% (C8orf37-W185X), 38.8% (PCCB-R111X), 22.1% (CBS-C275X), and 8.0% (CBS-W390X) EGFP positive cells were detected compared to positive controls, respectively (FIG. 14A). Next, PTC-readthrough of disease genes was tested for RESTARTv2 (DKC1-isoform3 overexpression) (FIG. 14B). DKC1-isoform3 overexpression (RESTARTv2) increased the relative fraction of EGFP positive cells (indicating PTC-readthrough) by an average of ˜2.8-fold compared to RESTARTv1. For cells expressing ALDOB-W148X and CBS-W390X, the relative fraction of EGFP positive cells were greatly increased with DKC1-isoform3 overexpression (by 4.8- and 6.3-fold, respectively), as shown in FIGS. 14A-14B).

RESTART was further validated for suppression of disease-relevant PTCs LMNA-R225X (associated with familial dilated cardiomyopathy (DCM) with conduction disease (DCM-CD)), F9-Y22X and F9-G21X (associated with hemophelia B), ABCA4-R408X (associated with Starfardt disease), RS1-Y65X (associated with X-linked retinoschisis), and Rpe65-R44X (associated with leber congenital amaurosis), as shown in FIG. 14C.

Finally, restoration of protein function using RESTART was demonstrated for a CFTR CFTR (cystic fibrosis transmembrane conductance regulator) gene containing a disease-relevant PTC. Mutations in CFTR cause the monogenetic disease cystic fibrosis, which affects approximately 1:2500 live births in caucasians. The ability of RESTART to repair the CFTR R553X (CGA-TGA) and W1282X (TGG-TGA) PTC sites and restore protein function was tested by electrophysiological assays, which is the “gold standard” for evaluating CFTR functional rescue. After delivery of RESTART, the function of CFTR containing PTC could be rescued to about 30% of WT CFTR level, indicating the therapeutic potential of RESTART in targeting certain monogenetic diseases.

### Example 7. Delivery of RESTART by Clinically Relevant Formats of gsnoRNAs

This example demonstrates the design and synthesis of functional oligonucleotides for gsnoRNA delivery to cells.

Full-length gsnoRNA oligonucleotides were prepared by in vitro transcription (IVT). To increase the stability of the gsnoRNA oligonucleotides in cells, a 5′ Cap modification (m⁷G(5′)ppp(5′)G cap analog) was added to the gsnoRNA oligonucleotides. The 5′ Cap modification is not present in endogenous intronic snoRNA. As an example, a 5′ Cap modified full-length gACA19 oligonucleotide targeting Reporter-2 (rACA19) was prepared by in vitro transcription (FIG. 15A-C). Of note, rACA19 increased the efficiency of PTC-readthrough for both RESTARTv1 and RESTARTv2, compared to a gACA19 expression construct vector) (FIG. 15D; data shown as mean±standard deviation).

Chemically synthesized half rACA19 oligonucleotides with 2′-O-methyl and phosphorothioate linkage modifications were prepared and tested for their ability to achieve efficient PTC-readthrough in cells, as shown in FIG. 15E (“P” indicates phosphorothioate linkages and “2′ O-methyl” indicates 2′ O-methyl modified nucleosides). The gsnoRNAs were delivered to cells by transfection.

Advantageously, the half gsnoRNA oligonucleotides facilitate chemical synthesis compared to the full-length gsnoRNA (˜130 nt), which is too long synthesized efficiently. Furthermore, the rH5 and rH3 oligonucleotides were synthesized with only six phosphorothioate linkages and four 2′ O-methyl modifications per oligonucleotide, indicating that a small number of modifications is sufficient to promote stability and function of the chemically synthesized half gsnoRNAs. The 5′ hairpin (gH5, with H box) and 3′ hairpin (gH3, with ACA box) constructs reduced the efficiency of PTC-readthrough compared to the gACA19 oligonucleotide prepared by IVT. However, both the rH5 and rH3 oligonucleotides, which have the same sequences as gH5 and gH3, exhibited comparable efficiency with the full-length gACA19 construct (FIG. 15D).

These results indicate that a gsnoRNA can be effectively delivered to cells as a full-length RNA oligonucleotide prepared by in vitro transcription (e.g., with a 5′ cap to increase stability), or as a half oligonucleotide comprising the 5′ hairpin or the 3′ hairpin prepared by chemical synthesis. Moreover, the data demonstrate that chemically synthesized rH3 or rH5 with six phosphorothioate linkages and only four 2′ O-methyl modifications are stable and functional in cells. Advantageously, the use of chemically synthesized rH3 and rH5 oligonucleotides with a small number of modifications can reducing the cost of preparing the chemically synthesized oligonucleotides. The delivered RNA oligonucleotides can function better than the same construct delivered to cells as a DNA vector encoding the same gsnoRNA construct.

### REFERENCES

1. Dieci, G., Preti, M. & Montanini, B. Eukaryotic snoRNAs: a paradigm for gene expression flexibility. Genomics 94, 83-8 (2009).
2. Jorjani, H. et al. An updated human snoRNAome. Nucleic Acids Res 44, 5068-82 (2016)
3. Gruber, A. R., Lorenz, R., Bernhart, S. H., Neuböck, R. & Hofacker, I. L. The Vienna RNA websuite. Nucleic Acids Res 36, W70-4 (2008).
4. Angrisani, A., Turano, M., Paparo, L., Di Mauro, C. & Furia, M. A new human dyskerin isoform with cytoplasmic localization. Biochim Biophys Acta 1810, 1361-8 (2011).
5. Dabrowski, M., Bukowy-Bieryllo, Z. & Zietkiewicz, E. Translational readthrough potential of natural termination codons in eukaryotes—The impact of RNA sequence. RNA Biol 12, 950-8 (2015).
6. Lei, Z. & Yi, C. A Radiolabeling-Free, qPCR-Based Method for Locus-Specific Pseudouridine Detection. Angew Chem Int Ed Engl 56, 14878-14882 (2017).

What is claimed is:

1. A method for editing a target RNA in a host cell, comprising introducing an engineered guide small nucleolar RNA (gsnoRNA) and a nucleic acid molecule encoding a DKC1 protein into the host cell, wherein the gsnoRNA comprises a guide sequence that hybridizes to a sequence comprising a target uridine residue in the target RNA, and wherein the gsnoRNA recruits the DKC1 protein to modify the target uridine residue into a pseudouridine residue in the target RNA.

2. A method for editing a target RNA in a host cell, comprising introducing an engineered gsnoRNA into the host cell, wherein the gsnoRNA comprises a guide sequence that hybridizes to a sequence comprising a target uridine residue in the target RNA, wherein the gsnoRNA comprises a scaffold sequence derived from a wildtype H/ACA-snoRNA selected from the group consisting of ACA2b, ACA36, ACA44, ACA27, E2, ACA3, and ACA17, and wherein the gsnoRNA recruits a DKC1 protein in the host cell to modify the target uridine residue into a pseudouridine residue in the target RNA.

3. A method for editing a target RNA in a host cell, comprising introducing an engineered gsnoRNA into the host cell, wherein the gsnoRNA comprises a guide sequence that hybridizes to a sequence comprising a target uridine residue in the target RNA, wherein the gsnoRNA comprises a nucleotide sequence selected from the group consisting of SEQ ID NOs: 4-6, 9-12, 15-19, 22-36, and 177-179, and wherein the gsnoRNA recruits a DKC1 protein in the host cell to modify the target uridine residue into a pseudouridine residue in the target RNA.

4. The method of claim 2 or 3, further comprising introducing a nucleic acid encoding the DKC1 protein into the host cell.

5. The method of claim 2 or 3, wherein the DKC1 protein is an endogenous DKC1 protein of the host cell.

6. The method of any one of claims 1-5, wherein the DKC1 protein has cytoplasmic localization in the host cell.

7. The method of any one of claims 1-6, wherein the DKC1 protein comprises a DKC1 protein fragment corresponding to amino acid residues 41 to 420 of a human DKC1 isoform 3 protein, wherein the amino acid numbering is according to SEQ ID NO: 2.

8. The method of any one of claims 1-7, wherein the DKC1 protein comprises an amino acid sequence having at least 85% identity to SEQ ID NO: 88.

9. The method of any one of claims 1-8, wherein the DKC1 protein comprises a naturally occurring DKC1 isoform with cytoplasmic localization in the host cell.

10. A method for editing a target RNA in a host cell, comprising introducing: (a) an engineered gsnoRNA and (b) a splice-switching antisense oligonucleotide (ASO) into the host cell, wherein the gsnoRNA comprises a guide sequence that hybridizes to a sequence comprising a target uridine residue in the target RNA, wherein the ASO enhances expression of a DKC1 protein that is an endogenous DKC1 isoform with cytoplasmic localization in the host cell, and wherein the gsnoRNA recruits the DKC1 protein to modify the target uridine residue into a pseudouridine residue in the target RNA.

11. The method of claim 9 or 10, wherein the DKC1 isoform corresponds to isoform 3 of human DKC1 protein.

12. The method of any one of claims 1-11, wherein the DKC1 protein comprises an amino acid sequence having at least 85% identity to SEQ ID NO: 2.

13. The method of any one of claims 1-12, wherein the target RNA is not a ribosomal RNA (rRNA).

14. The method of any one of claims 1 and 4-13, wherein the gsnoRNA comprises a scaffold sequence derived from a wildtype H/ACA-snoRNA selected from the group consisting of ACA19, ACA2b, ACA36, ACA44, ACA27, E2, ACA3, and ACA17.

15. The method of claim 2 or 14, wherein the gsnoRNA comprises a scaffold sequence derived from ACA2b.

16. The method of claim 2 or 14, wherein the gsnoRNA comprises a scaffold sequence derived from ACA36.

17. The method of claim 16, wherein the gsnoRNA comprises a mutation in the 3′ hairpin of the ACA36 scaffold.

18. The method of claim 14, wherein the gsnoRNA comprises a scaffold sequence derived from ACA19.

19. The method of any one of claims 14-18, wherein the gsnoRNA comprises one or more guide sequences each located in a region corresponding to a hairpin structure of the wildtype H/ACA-snoRNA.

20. The method of claim 19, wherein at least one of the one or more guide sequences is located in a hairpin structure at the 3′ terminal part of the wildtype H/ACA-snoRNA.

21. The method of claim 19 or 20, wherein at least one of the one or more guide sequences is located in a hairpin structure at the 5′ terminal part of the wildtype H/ACA-snoRNA.

22. The method of any one of claims 17-21, wherein the gsnoRNA comprises one or more mutations in one or more hairpin structures of the wildtype ACA19.

23. The method of any one of claims 1-22, wherein the engineered gsnoRNA comprises one or more substitution mutations in nucleotides of a polyU sequence in the wildtype H/ACA-snoRNA, wherein the polyU sequence comprises at least 4 consecutive U residues.

24. The method of any one of claims 1-23, wherein the engineered gsnoRNA comprises one or more insertion or deletion mutations positioned between the nucleotide residue in the guide region that hybridizes to the target uridine and an H/ACA box of the wildtype H/ACA snoRNA, whereby the engineered gsnoRNA comprises 14 or 15 nucleotides between the nucleotide residue in the guide region that hybridizes to the target uridine and the H/ACA box.

25. The method of claim 22, wherein the one or more mutations are selected from the group consisting of substitution of residues 26-29 with UUCU, substitution of residues 26-29 with UGUU, addition of G to the 3′ hairpin structure after residue 115, and addition of CU to the 5′ hairpin after residue 8, and wherein the numbering is according to SEQ ID NO: 37.

26. The method of any one of claims 3-4 and 14-25, wherein the gsnoRNA comprises a nucleotide sequence selected from the group consisting of SEQ ID NOs: 3-12, 15-19, 22-36, and 177-179.

27. The method of claim 4 or 26, wherein the gsnoRNA comprises a nucleotide sequence selected from the group consisting of SEQ ID NOs: 15-19.

28. The method of any one of claims 1-27, wherein the method comprises introducing a nucleic acid molecule encoding the gsnoRNA into the host cell.

29. The method of claim 28, wherein the nucleic acid molecule encoding the gsnoRNA is under a promoter selected from the group consisting of U6 promoter and U1 promoter.

30. The method of claim 28 or 29, wherein the nucleic acid molecule encoding the gsnoRNA is embedded in an intron sequence located between a first exon sequence and a second exon sequence, and wherein the first exon sequence, the intron sequence and the second exon sequence are derived from a naturally-occurring gene.

31. The method of any one of claims 1, 4 and 28-30, wherein the nucleic acid molecule encoding the DKC1 protein and/or the nucleic acid molecule encoding the gsnoRNA are present in a viral vector.

32. The method of claim 1 or 4, wherein the method comprises introducing into the host cell a vector comprising a first nucleic acid sequence encoding the DKC1 protein and a second nucleic acid sequence encoding the gsnoRNA.

33. The method of claim 32, wherein the vector is a viral vector.

34. The method of claim 32 or 33, wherein the vector is an adeno-associated viral (AAV) vector.

35. The method of any one of claims 1-27, wherein the gsnoRNA comprises one or more chemically modified nucleosides and/or inter-nucleosidic linkages.

36. The method of claim 35, wherein the gsnoRNA comprises one or more nucleosides having 2′-OMe or 2′-MOE modifications.

37. The method of claim 35 or 36, wherein the gsnoRNA comprises no more than 10, no more than 8, no more than 6, or no more than 4 chemically modified nucleosides.

38. The method of any one of claims 35-37, wherein the gsnoRNA comprises one or more phosphorothioate inter-nucleosidic linkages.

39. The method of any one of claims 35-38, wherein the gsnoRNA comprises no more than 10, no more than 9, no more than 8, or no more than 6 phosphorothioate inter-nucleosidic linkages.

40. The method of any one of claims 1-39, wherein the gsnoRNA comprises a 5′ cap modification.

41. The method of claim 40, wherein the S′ cap modification is a 7-methylguanosine (m⁷G) cap.

42. The method of any one of claims 1-38, wherein efficiency of editing the target RNA is at least 10%.

43. The method of any of claims 1-42, wherein the target RNA is mRNA.

44. The method of any one of claims 1-43, wherein the sequence comprising the target uridine in the target RNA is a stop codon, and wherein modification of the target uridine to pseudouridine causes the stop codon to be translated as a coding codon.

45. The method of claim 44, wherein the stop codon is a premature termination codon (PTC).

46. The method of claim 45, wherein the PTC is associated with a genetic disease or condition.

47. The method of any one of claims 1-46, wherein the DKC1 protein is part of a ribonucleoprotein (RNP) complex that associates with the gsnoRNA.

48. The method of any one of claims 1-47, wherein the host cell is an archaeal or eukaryotic cell.

49. The method of claim 48, wherein the host cell is a mammalian cell.

50. The method of claim 49, wherein the host cell is a human cell.

51. The method of any one of claims 1-50, wherein the method is carried out in vivo.

52. The method of any one of claims 1-51, wherein the method is carried out ex vivo.

53. A method of treating a disease or condition associated with a PTC in a target RNA in a subject, comprising editing the target RNA in a cell of the subject using the method of any one of claims 1-52, wherein the gsnoRNA comprises a guide sequence that hybridizes to the PTC in the target RNA, and wherein modification of the uridine residue in the PTC to a pseudouridine residue causes translation read-through of the PTC in the target RNA, thereby treating the disease or condition in the subject.

54. The method of claim 53, wherein the disease or condition is selected from the group consisting of Cystic fibrosis, Hurler Syndrome, alpha-1-antitrypsin (A1AT) deficiency, Parkinson's disease, Alzheimer's disease, albinism, Amyotrophic lateral sclerosis, Asthma, 8-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase, Haemophilia, Hereditary Hemochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer.

55. An engineered gsnoRNA comprising a guide sequence that hybridizes to a sequence comprising a target uridine residue in a target RNA in a host cell, wherein the gsnoRNA comprises a nucleotide sequence selected from the group consisting of SEQ ID NOs: 4-6, 9-12, 15-19,22-36, and 177-179, and wherein the gsnoRNA is capable of recruiting a DKC1 protein in the host cell to modify the target uridine residue into a pseudouridine residue in the target RNA.

56. An engineered gsnoRNA comprising a guide sequence that hybridizes to a sequence comprising a target uridine residue in a target RNA in a host cell, wherein the gsnoRNA comprises a scaffold sequence derived from a wildtype H/ACA-snoRNA selected from the group consisting of ACA2b, ACA36, ACA44, ACA27, E2, ACA3, and ACA17, and wherein the gsnoRNA is capable of recruiting a DKC1 protein in the host cell to modify the target uridine residue into a pseudouridine residue in the target RNA.

57. The engineered gsnoRNA of any one of claims 55-56, wherein the gsnoRNA comprises a 5′ cap modification.

58. The engineered gsnoRNA of claim 57, wherein the 5′ cap modification is a 7-methylguanosine (m⁷G) cap.

59. The engineered gsnoRNA of any one of claims 55-58, wherein the gsnoRNA comprises one or more chemically modified nucleosides and/or inter-nucleosidic linkages.

60. The engineered gsnoRNA of any one of claims 55-59, wherein the gsnoRNA comprises one or more nucleosides having 2′-OMe or 2′-MOE modifications.

61. The engineered gsnoRNA of any one of claims 55-60, wherein the gsnoRNA comprises no more than 10, no more than 8, no more than 6, or no more than 4 chemically modified nucleosides.

62. The engineered gsnoRNA of any one of claims 55-61, wherein the gsnoRNA comprises one or more phosphorothioate inter-nucleosidic linkages.

63. The engineered gsnoRNA of claim 62, wherein the gsnoRNA comprises no more than 10, no more than 9, no more than 8, or no more than 6 phosphorothioate inter-nucleosidic linkages.

64. An isolated nucleic acid molecule comprising a sequence encoding the gsnoRNA of any one of claims 55-632.

65. An engineered RNA-editing system comprising:
(a) a gsnoRNA comprising a guide sequence that hybridizes to a sequence comprising a target uridine residue in a target RNA in a host cell, or a nucleic acid molecule encoding the gsnoRNA; and
(b) a DKC1 protein, or a nucleic acid molecule encoding the DKC1 protein, wherein the gsnoRNA is capable of recruiting the DKC1 protein to modify the target uridine residue into a pseudouridine residue in the target RNA.

66. A pharmaceutical composition comprising the gsnoRNA of any one of claims 55-63, the nucleic acid molecule of claim 64, or the engineered RNA-editing system of claim 65, and a pharmaceutically acceptable carrier.

67. A host cell comprising the gsnoRNA of any one of claims 55-63, the nucleic acid molecule of claim 64, or the engineered RNA-editing system of claim 65.

68. A kit for editing a target RNA in a host cell, comprising the gsnoRNA of any one of claims 55-63, the nucleic acid molecule of claim 64, or the engineered RNA-editing system of claim 65.


SEQUENCE LISTING (197 sequences)

SEQ ID NO: 1 - protein, 514 aa, Homo sapiens
MADAEVIILPKKHKKKKERKSLPEEDVAEIQHAEEFLIKPESKVAKLDTSQWPLLLKNFDKLNVRTTHYTPLACGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWKQEYVDYSESAKKEVVAEVVKAPQVVAEAAKTAKRKRESESESDETPPAAPQLIKKEKKKSKKDKKAKAGLESGAEPGDGDSDTTKKKKKKKKAKEVELVSE

SEQ ID NO: 2 - protein, 420 aa, Homo sapiens
MADAEVIILPKKHKKKKERKSLPEEDVAEIQHAEEFLIKPESKVAKLDTSQWPLLLKNFDKLNVRTTHYTPLACGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWKQEYVDYR

SEQ ID NO: 3 - RNA, 104 nt, Artificial Sequence
  Synthetic Construct
  misc_feature 8, 38, 62, 91: n = A, U, G, or C, and is present in repeats of 4, 5, 6, 7, 8, 9, 10, 11, or 12
gugcacangaccugcuuucuuuuaugugaguaguguungugcuauacaaauaauugaaggcngcaguauaacuauaaauaguaaugcugcnccuucagacaaaa

SEQ ID NO: 4 - RNA, 110 nt, Artificial Sequence
  Synthetic Construct
  misc_feature 6, 36, 61, 95: n = A, U, G, or C, and is present in repeats of 4, 5, 6, 7, 8, 9, 10, 11, or 12
cagcangggcuguggcuggucauagccaugggaucngcaugcaagagcaaccuggaaaganacagcgcaggucaguacaauaccugcaagcugcnagcuuuccuauaaug

SEQ ID NO: 5 - RNA, 98 nt, Artificial Sequence
  Synthetic Construct
  misc_feature 7, 35, 59, 86: n = A, U, G, or C, and is present in repeats of 4, 5, 6, 7, 8, 9, 10, 11, or 12
uaccccngccaguuggacuuaugucuuuauuggunaguggggcaaaggaaauauccuunucaggcaaacuggguguuugucuguangaggaaacaaau

SEQ ID NO: 6 - RNA, 128 nt, Artificial Sequence
  Synthetic Construct
  misc_feature 9, 52, 73, 112: n = A, U, G, or C, and is present in repeats of 4, 5, 6, 7, 8, 9, 10, 11, or 12
ugugcacangcuuggaguugaggcuacugacuggccgaugaacucgcaagungugcuacaugaggggcaagunacaccacaagggucucuggcccaaugaguggaguuuganauucuugcuacaagua

SEQ ID NO: 7 - RNA, 102 nt, Artificial Sequence
  Synthetic Construct
  misc_feature 5, 35, 60, 89: n = A, U, G, or C, and is present in repeats of 4, 5, 6, 7, 8, 9, 10, 11, or 12
cacangaccugcuuucuuuuaugugaguaguguunugugcuauacaaauaauugaaggcngcaguauaacuauaaauaguaaugcugcnccuucagacaaaa

SEQ ID NO: 8 - RNA, 122 nt, Artificial Sequence
  Synthetic Construct
  misc_feature 17, 47, 71, 100: n = A, U, G, or C, and is present in repeats of 4, 5, 6, 7, 8, 9, 10, 11, or 12
ucaguauuugugcacangaccugcuuucuuuuaugugaguaguguungugcuauacaaauaauugaaggcngcaguauaacuauaaauaguaaugcugcnccuucagacaaaaauucuauaa

SEQ ID NO: 9 - RNA, 106 nt, Artificial Sequence
  Synthetic Construct
  misc_feature 6, 37, 59, 90: n = A, U, G, or C, and is present in repeats of 4, 5, 6, 7, 8, 9, 10, 11, or 12
aucganacgcuuggguaucggcuauugccugagugunccucgaagaguaacugcugacnacuggcugugggccuuauggcacagucaguncagguuagagacaugc

SEQ ID NO: 10 - RNA, 103 nt, Artificial Sequence
  Synthetic Construct
  misc_feature 10, 39, 65, 92: n = A, U, G, or C, and is present in repeats of 4, 5, 6, 7, 8, 9, 10, 11, or 12
acugccccungcagcuguggcugccgugucacaucugunguggcagagauuagagaggcuauguncaagcguucugccccgugaacguuugngucucacacuc

SEQ ID NO: 11 - RNA, 116 nt, Artificial Sequence
  Synthetic Construct
  misc_feature 9, 38, 65, 102: n = A, U, G, or C, and is present in repeats of 4, 5, 6, 7, 8, 9, 10, 11, or 12
uuggcucunggccagcaguuugcugaagcuguuggccncaggagccuaaagaauugucuuucuanuuggccauuucauaacuuuggaaauguaauggucaanagaaagaaacauga

SEQ ID NO: 12 - RNA, 107 nt, Artificial Sequence
  Synthetic Construct
  misc_feature 8, 37, 66, 90: n = A, U, G, or C, and is present in repeats of 4, 5, 6, 7, 8, 9, 10, 11, or 12
uuccaaanucaguccagggcagcuucccuguucuganuuugggacauuaaaaugggcuaagggagnggguagaaaguauuauucuauucnccucccagccuacaaaa

SEQ ID NO: 13 - protein, 400 aa, Homo sapiens
MLPEEDVAEIQHAEEFLIKPESKVAKLDTSQWPLLLKNFDKLNVRTTHYTPLACGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWKQEYVDYR

SEQ ID NO: 14 - RNA, 132 nt, Artificial Sequence
  Synthetic Construct
cagcaagcaucgaggggcuguggcuggucauagccaugggaucguacuccgcaugcaagagcaaccuggaaagacagugacagcgcaggucaguacaauaccugcaagcugcaugccagcuuuccuauaaug

SEQ ID NO: 15 - RNA, 104 nt, Artificial Sequence
  Synthetic Construct
  misc_feature 8, 38, 62, 91: n = A, U, G, or C, and is present in repeats of 4, 5, 6, 7, 8, 9, 10, 11, or 12
gugcacangaccugcuuucuucuaugugaguaguguungugcuauacaaauaauugaaggcngcaguauaacuauaaauaguaaugcugcnccuucagacaaaa

SEQ ID NO: 16 - RNA, 104 nt, Artificial Sequence
  Synthetic Construct
  misc_feature 8, 38, 62, 91: n = A, U, G, or C, and is present in repeats of 4, 5, 6, 7, 8, 9, 10, 11, or 12
gugcacangaccugcuuucuguuaugugaguaguguungugcuauacaaauaauugaaggcngcaguauaacuauaaauaguaaugcugcnccuucagacaaaa

SEQ ID NO: 17 - RNA, 105 nt, Artificial Sequence
  Synthetic Construct
  misc_feature 8, 38, 62, 91: n = A, U, G, or C, and is present in repeats of 4, 5, 6, 7, 8, 9, 10, 11, or 12
gugcacangaccugcuuucuuuuaugugaguaguguungugcuauacaaauaauugaaggcngcaguauaacuauaaauaguaaugcugcngccuucagacaaaa

SEQ ID NO: 18 - RNA, 105 nt, Artificial Sequence
  Synthetic Construct
  misc_feature 8, 38, 63, 92: n = A, U, G, or C, and is present in repeats of 4, 5, 6, 7, 8, 9, 10, 11, or 12
gugcacangaccugcuuucuuuuaugugaguaguguungugcuauacaaauaauugaaggcungcaguauaacuauaaauaguaaugcugcnccuucagacaaaa

SEQ ID NO: 19 - RNA, 107 nt, Artificial Sequence
  Synthetic Construct
  misc_feature 11, 41, 65, 94: n = A, U, G, or C, and is present in repeats of 44, 5, 6, 7, 8, 9, 10, 11, or 12
gugcacaucungaccugcuuucuuuuaugugaguaguguungugcuauacaaauaauugaaggcngcaguauaacuauaaauaguaaugcugcnccuucagacaaaa

SEQ ID NO: 20 - RNA, 117 nt, Artificial Sequence
  Synthetic Construct
  misc_feature 10, 39, 66, 103: n = A, U, G, or C, and is present in repeats of 4, 5, 6, 7, 8, 9, 10, 11, or 12
uuggcucucnggccagcaguuugcugaagcuguuggccncaggagccuaaagaauugucuuucuanuuggccauuucauaacuuuggaaauguaauggucaanagaaagaaacauga

SEQ ID NO: 21 - RNA, 114 nt, Artificial Sequence
  Synthetic Construct
  misc_feature 7, 36, 63, 100: n = A, U, G, or C, and is present in repeats of 4, 5, 6, 7, 8, 9, 10, 11, or 12
uuggcunggccagcaguuugcugaagcuguuggccncaggagccuaaagaauugucuuucuanuuggccauuucauaacuuuggaaauguaauggucaanagaaagaaacauga

SEQ ID NO: 22 - RNA, 107 nt, Artificial Sequence
  Synthetic Construct
  misc_feature 8, 37, 66, 90: n = A, U, G, or C, and is present in repeats of 4, 5, 6, 7, 8, 9, 10, 11, or 12
uuccaaanucaguccagggcagcuucccuguacuganuuugggacauuaaaaugggcuaagggagnggguagaaaguauuauucuauucnccucccagccuacaaaa

SEQ ID NO: 23 - RNA, 107 nt, Artificial Sequence
  Synthetic Construct
  misc_feature 8, 37, 66, 90: n = A, U, G, or C, and is present in repeats of 4, 5, 6, 7, 8, 9, 10, 11, or 12
uuccaaanucaguccagggcagcuucccuggacuganuuugggacauuaaaaugggcuaagggagnggguagaaaguauuauucuauucnccucccagccuacaaaa

SEQ ID NO: 24 - RNA, 107 nt, Artificial Sequence
  Synthetic Construct
  misc_feature 8, 37, 66, 90: n = A, U, G, or C, and is present in repeats of 4, 5, 6, 7, 8, 9, 10, 11, or 12
uuccaagnucaguccagggcagcuucccuguucugancuugggacauuaaaaugggcuaagggagnggguagaaaguauuauucuauucnccucccagccuacaaaa

SEQ ID NO: 25 - RNA, 106 nt, Artificial Sequence
  Synthetic Construct
  misc_feature 8, 37, 65, 89: n = A, U, G, or C, and is present in repeats of 4, 5, 6, 7, 8, 9, 10, 11, or 12
uuccaaanucaguccagggcagcuucccuguucuganuuugggacauuaaaaugggcuaaggganggguagaaaguauuauucuauucnccucccagccuacaaaa

SEQ ID NO: 26 - RNA, 104 nt, Artificial Sequence
  Synthetic Construct
  misc_feature 8, 37, 63, 87: n = A, U, G, or C, and is present in repeats of 4, 5, 6, 7, 8, 9, 10, 11, or 12
uuccaaanucaguccagggcagcuucccuguucuganuuugggacauuaaaaugggcuggganggguagaaaguauuauucuauucnccucccagccuacaaaa

SEQ ID NO: 27 - RNA, 105 nt, Artificial Sequence
  Synthetic Construct
  misc_feature 8, 37, 64, 88: n = A, U, G, or C, and is present in repeats of 4, 5, 6, 7, 8, 9, 10, 11, or 12
uuccaaanucaguccagggcagcuucccuguucuganuuugggacauuaaaaugggcuaggganggguagaaaguauuauucuauucnccucccagccuacaaaa

SEQ ID NO: 28 - RNA, 107 nt, Artificial Sequence
  Synthetic Construct
  misc_feature 8, 37, 66, 90: n = A, U, G, or C, and is present in repeats of 4, 5, 6, 7, 8, 9, 10, 11, or 12
uuccaaanucaguccagggcagcuucccuguucuganuuugggacauuaaaaugggcuaagggagnggguagaaaguauuauucuauccnccucccagccuacaaaa

SEQ ID NO: 29 - RNA, 104 nt, Artificial Sequence
  Synthetic Construct
  misc_feature 8, 37, 63, 87: n = A, U, G, or C, and is present in repeats of 4, 5, 6, 7, 8, 9, 10, 11, or 12
uuccaaanucaguccagggcagcuucccuguucuganuuugggacauuaaaaugggcuggganggguagaaaguauuauucuauccnccucccagccuacaaaa

SEQ ID NO: 30 - RNA, 105 nt, Artificial Sequence
  Synthetic Construct
  misc_feature 8, 37, 65, 89: n = A, U, G, or C, and is present in repeats of 4, 5, 6, 7, 8, 9, 10, 11, or 12
uuccaaanucaguccagggcagcuucccuguucuganuuugggacauuaaaauggcuaagggagnggguagaaaguauuauucuauucnccucccagcuacaaaa

SEQ ID NO: 31 - RNA, 106 nt, Artificial Sequence
  Synthetic Construct
  misc_feature 8, 37, 66, 90: n = A, U, G, or C, and is present in repeats of 4, 5, 6, 7, 8, 9, 10, 11, or 12
uuccaaanucaguccagggcagcuucccuguucuganuuugggacauuaaaaugggcuaagggagnggguagaaaguauuauucuauucncucccagccuacaaaa

SEQ ID NO: 32 - RNA, 105 nt, Artificial Sequence
  Synthetic Construct
  misc_feature 8, 37, 65, 89: n = A, U, G, or C, and is present in repeats of 4, 5, 6, 7, 8, 9, 10, 11, or 12
uuccaaanucaguccagggcagcuucccuguucuganuuugggacauuaaaaugggcuaaggganggguagaaaguauuauucuauucncucccagccuacaaaa

SEQ ID NO: 33 - RNA, 105 nt, Artificial Sequence
  Synthetic Construct
  misc_feature 8, 38, 63, 92: n = A, U, G, or C, and is present in repeats of 4, 5, 6, 7, 8, 9, 10, 11, or 12
gugcacangaccugcuuucuucuaugugaguaguguunugugcuauacaaauaauugaaggcngcaguauaacuauaaauaguaaugcugcnccuucagacaaaa

SEQ ID NO: 34 - RNA, 105 nt, Artificial Sequence
  Synthetic Construct
  misc_feature 9, 39, 63, 92: n = A, U, G, or C, and is present in repeats of 4, 5, 6, 7, 8, 9, 10, 11, or 12
gugcacaungaccugcuuucuucuaugugaguaguguungugcuauacaaauaauugaaggcngcaguauaacuauaaauaguaaugcugcnccuucagacaaaa

SEQ ID NO: 35 - RNA, 105 nt, Artificial Sequence
  Synthetic Construct
  misc_feature 9, 39, 63, 92: n = A, U, G, or C, and is present in repeats of 4, 5, 6, 7, 8, 9, 10, 11, or 12
gugcacaungaccugcuuucuuuuaugugaguaguguungugcuauacaaauaauugaaggcngcaguauaacuauaaauaguaaugcugcnccuucagacaaaa

SEQ ID NO: 36 - RNA, 106 nt, Artificial Sequence
  Synthetic Construct
  misc_feature 10, 40, 64, 93: n = A, U, G, or C, and is present in repeats of 4, 5, 6, 7, 8, 9, 10, 11, or 12
gugcacaucngaccugcuuucuucuaugugaguaguguungugcuauacaaauaauugaaggcngcaguauaacuauaaauaguaaugcugcnccuucagacaaaa

SEQ ID NO: 37 - RNA, 128 nt, Artificial Sequence
  Synthetic Construct
gugcacaugaucccgaccugcuuucuuuuaugugaguaguguuuccggugaugugcuauacaaauaauugaaggcgaucccgcaguauaacuauaaauaguaaugcugcuccgguccuucagacaaaa

SEQ ID NO: 38 - RNA, 132 nt, Artificial Sequence
  Synthetic Construct
cagcagcugaucccgggcuguggcuggucauagccaugggaucuccgguggcaugcaagagcaaccuggaaagaaucccacagcgcaggucaguacaauaccugcaagcugcuccggagcuuuccuauaaug

SEQ ID NO: 39 - RNA, 126 nt, Artificial Sequence
  Synthetic Construct
uacccccggcugaucccgccaguuggacuuaugucuuuauugguuccgguaguggggcaaaggaaauauccuuugaucccucaggcaaacuggguguuugucuguauccggugagaggaaacaaau

SEQ ID NO: 40 - RNA, 154 nt, Artificial Sequence
  Synthetic Construct
ugugcacacugaucccgcuuggaguugaggcuacugacuggccgaugaacucgcaaguuccggugaugugcuacaugaggggcaagucugaucccacaccacaagggucucuggcccaaugaguggaguuugauccggauucuugcuacaagua

SEQ ID NO: 41 - RNA, 125 nt, Artificial Sequence
  Synthetic Construct
cacaugaucccgaccugcuuucuuuuaugugaguaguguuuccggugaugugcuauacaaauaauugaaggcgaucccgcaguauaacuauaaauaguaaugcugcuccgguccuucagacaaaa

SEQ ID NO: 42 - RNA, 146 nt, Artificial Sequence
  Synthetic Construct
ucaguauuugugcacaugaucccgaccugcuuucuuuuaugugaguaguguuuccggugaugugcuauacaaauaauugaaggcgaucccgcaguauaacuauaaauaguaaugcugcuccgguccuucagacaaaaauucuauaa

SEQ ID NO: 43 - RNA, 131 nt, Artificial Sequence
  Synthetic Construct
aucgaggcugaucccacgcuuggguaucggcuauugccugaguguuccggugaccucgaagaguaacugcugacugaucccacuggcugugggccuuauggcacagucaguuccgcagguuagagacaugc

SEQ ID NO: 44 - RNA, 132 nt, Artificial Sequence
  Synthetic Construct
acugccccucugaucccgcagcuguggcugccgugucacaucuguuccggugaguggcagagauuagagaggcuauguugauccccaagcguucugccccgugaacguuuguccggugauagucucacacuc

SEQ ID NO: 45 - RNA, 137 nt, Artificial Sequence
  Synthetic Construct
uuggcucuugaucccggccagcaguuugcugaagcuguuggccuccggcaggagccuaaagaauugucuuucuaugaucccuuggccauuucauaacuuuggaaauguaauggucaauccgguagaaagaaacauga

SEQ ID NO: 46 - RNA, 130 nt, Artificial Sequence
  Synthetic Construct
uuccaaagcugaucccucaguccagggcagcuucccuguucugauccggugauuugggacauuaaaaugggcuaagggaggaucccggguagaaaguauuauucuauucuccgccucccagccuacaaaa

SEQ ID NO: 47 - RNA, 128 nt, Artificial Sequence
  Synthetic Construct
gugcacaugugcuagaccugcuuucuuuuaugugaguaguguugcuguuaaugugcuauacaaauaauugaaggcgaucccgcaguauaacuauaaauaguaaugcugcuccgguccuucagacaaaa

SEQ ID NO: 48 - RNA, 128 nt, Artificial Sequence
  Synthetic Construct
gugcacaugaucccgaccugcuuucuuuuaugugaguaguguuuccggugaugugcuauacaaauaauugaaggcgaccgggcaguauaacuauaaauaguaaugcugcagcuauccuucagacaaaa

SEQ ID NO: 49 - RNA, 128 nt, Artificial Sequence
  Synthetic Construct
gugcacaugaucccgaccugcuuucuucuaugugaguaguguuuccggugaugugcuauacaaauaauugaaggcgaucccgcaguauaacuauaaauaguaaugcugcuccgguccuucagacaaaa

SEQ ID NO: 50 - RNA, 128 nt, Artificial Sequence
  Synthetic Construct
gugcacaugaucccgaccugcuuucuguuaugugaguaguguuuccggugaugugcuauacaaauaauugaaggcgaucccgcaguauaacuauaaauaguaaugcugcuccgguccuucagacaaaa

SEQ ID NO: 51 - RNA, 129 nt, Artificial Sequence
  Synthetic Construct
gugcacaugaucccgaccugcuuucuuuuaugugaguaguguuuccggugaugugcuauacaaauaauugaaggcgaucccgcaguauaacuauaaauaguaaugcugcuccggugccuucagacaaaa

SEQ ID NO: 52 - RNA, 130 nt, Artificial Sequence
  Synthetic Construct
gugcacaugaucccgaccugcuuucuuuuaugugaguaguguuuccggugaugugcuauacaaauaauugaaggcugaucccgcaguauaacuauaaauaguaaugcugcuccggugccuucagacaaaa

SEQ ID NO: 53 - RNA, 130 nt, Artificial Sequence
  Synthetic Construct
gugcacaucugaucccgaccugcuuucuuuuaugugaguaguguuuccggugaugugcuauacaaauaauugaaggcgaucccgcaguauaacuauaaauaguaaugcugcuccgguccuucagacaaaa

SEQ ID NO: 54 - RNA, 130 nt, Artificial Sequence
  Synthetic Construct
uuccaaagcucuaagaucaguccagggcagcuucccuguucugaguaauugauuugggacauuaaaaugggcuaagggaggaucccggguagaaaguauuauucuauucuccgccucccagccuacaaaa

SEQ ID NO: 55 - RNA, 130 nt, Artificial Sequence
  Synthetic Construct
uuccaaagcugaucccucaguccagggcagcuucccuguucugauccggugauuugggacauuaaaaugggcuaagggagguaagaggguagaaaguauuauucuauucguauccucccagccuacaaaa

SEQ ID NO: 56 - RNA, 130 nt, Artificial Sequence
  Synthetic Construct
uuccaaagcugaucccucaguccagggcagcuucccuguacugauccggugauuugggacauuaaaaugggcuaagggaggaucccggguagaaaguauuauucuauucuccgccucccagccuacaaaa

SEQ ID NO: 57 - RNA, 130 nt, Artificial Sequence
  Synthetic Construct
uuccaaagcugaucccucaguccagggcagcuucccuggacugauccggugauuugggacauuaaaaugggcuaagggaggaucccggguagaaaguauuauucuauucuccgccucccagccuacaaaa

SEQ ID NO: 58 - RNA, 130 nt, Artificial Sequence
  Synthetic Construct
uuccaaggcugaucccucaguccagggcagcuucccuguucugauccggugacuugggacauuaaaaugggcuaagggaggaucccggguagaaaguauuauucuauucuccgccucccagccuacaaaa

SEQ ID NO: 59 - RNA, 130 nt, Artificial Sequence
  Synthetic Construct
uuccaaagcugaucccucaguccagggcagcuucccuguucugauccggugauuugggacauuaaaaugggcuaagggaugaucccggguagaaaguauuauucuauucuccgccucccagccuacaaaa

SEQ ID NO: 60 - RNA, 128 nt, Artificial Sequence
  Synthetic Construct
uuccaaagcugaucccucaguccagggcagcuucccuguucugauccggugauuugggacauuaaaaugggcugggaugaucccggguagaaaguauuauucuauucuccgccucccagccuacaaaa

SEQ ID NO: 61 - RNA, 129 nt, Artificial Sequence
  Synthetic Construct
uuccaaagcugaucccucaguccagggcagcuucccuguucugauccggugauuugggacauuaaaaugggcuagggaugaucccggguagaaaguauuauucuauucuccgccucccagccuacaaaa

SEQ ID NO: 62 - RNA, 130 nt, Artificial Sequence
  Synthetic Construct
uuccaaagcugaucccucaguccagggcagcuucccuguucugauccggugauuugggacauuaaaaugggcuaagggaggaucccggguagaaaguauuauucuauccuccgccucccagccuacaaaa

SEQ ID NO: 63 - RNA, 128 nt, Artificial Sequence
  Synthetic Construct
uuccaaagcugaucccucaguccagggcagcuucccuguucugauccggugauuugggacauuaaaaugggcugggaugaucccggguagaaaguauuauucuauccuccgccucccagccuacaaaa

SEQ ID NO: 64 - RNA, 128 nt, Artificial Sequence
  Synthetic Construct
uuccaaagcugaucccucaguccagggcagcuucccuguucugauccggugauuugggacauuaaaauggcuaagggaggaucccggguagaaaguauuauucuauucuccgccucccagcuacaaaa

SEQ ID NO: 65 - RNA, 129 nt, Artificial Sequence
  Synthetic Construct
uuccaaagcugaucccucaguccagggcagcuucccuguucugauccggugauuugggacauuaaaaugggcuaagggaggaucccggguagaaaguauuauucuauucuccgcucccagccuacaaaa

SEQ ID NO: 66 - RNA, 129 nt, Artificial Sequence
  Synthetic Construct
uuccaaagcugaucccucaguccagggcagcuucccuguucugauccggugauuugggacauuaaaaugggcuaagggaugaucccggguagaaaguauuauucuauucuccgcucccagccuacaaaa

SEQ ID NO: 67 - RNA, 129 nt, Artificial Sequence
  Synthetic Construct
gugcacaugaucccgaccugcuuucuucuaugugaguaguguuuccggugaugugcuauacaaauaauugaaggcgaucccgcaguauaacuauaaauaguaaugcugcuccggugccuucagacaaaa

SEQ ID NO: 68 - RNA, 130 nt, Artificial Sequence
  Synthetic Construct
gugcacaucugaucccgaccugcuuucuucuaugugaguaguguuuccggugaugugcuauacaaauaauugaaggcgaucccgcaguauaacuauaaauaguaaugcugcuccgguccuucagacaaaa

SEQ ID NO: 69 - RNA, 131 nt, Artificial Sequence
  Synthetic Construct
gugcacaucugaucccgaccugcuuucuuuuaugugaguaguguuuccggugaugugcuauacaaauaauugaaggcgaucccgcaguauaacuauaaauaguaaugcugcuccggugccuucagacaaaa

SEQ ID NO: 70 - RNA, 131 nt, Artificial Sequence
  Synthetic Construct
gugcacaucugaucccgaccugcuuucuucuaugugaguaguguuuccggugaugugcuauacaaauaauugaaggcgaucccgcaguauaacuauaaauaguaaugcugcuccggugccuucagacaaaa

SEQ ID NO: 71 - RNA, 130 nt, Artificial Sequence
  Synthetic Construct
gugcacaucggcacgugaccugcuuucuucuaugugaguaguguucuucccaaugugcuauacaaauaauugaaggcgcacgugcaguauaacuauaaauaguaaugcugccuuccuccuucagacaaaa

SEQ ID NO: 72 - RNA, 132 nt, Artificial Sequence
  Synthetic Construct
uuccaaagcggcacguucaguccagggcagcuucccuguucugauuuccuaauuugggacauuaaaaucggcuggugagcacguggacuaagaaaguauuauucauagucccuucccgaccagccuacaaaa

SEQ ID NO: 73 - RNA, 130 nt, Artificial Sequence
  Synthetic Construct
gugcacauaagaguuugaccugcuuucuucuaugugaguaguguuuggaguaaugugcuauacaaauaauugaaggcgaguuugcaguauaacuauaaauaguaaugcugcuggaguccuucagacaaaa

SEQ ID NO: 74 - RNA, 132 nt, Artificial Sequence
  Synthetic Construct
uuccaaagaagaguuuacaguccagggcagcuucccuguucuguuggaguaguuugggacauuaaaaucggcuggugagaguuuggacuaagaaaguauuauucauaguccuggagcgaccagccuacaaaa

SEQ ID NO: 75 - RNA, 130 nt, Artificial Sequence
  Synthetic Construct
gugcacauugguucuugaccugcuuucuucuaugugaguaguguugcuauacaugugcuauacaaauaauugaaggaguucuugcaguauaacuauaaauaguaaugcugcgcuauaccuucagacaaaa

SEQ ID NO: 76 - RNA, 130 nt, Artificial Sequence
  Synthetic Construct
uuccaaacuaguucuuucaguccagggcagcuucccuguucugagcuacacauuugggacauuaaaaugggcuaaggguaguucuuggguagaaaguauuauucuauucgcuauaucccagccuacaaaa

SEQ ID NO: 77 - RNA, 130 nt, Artificial Sequence
  Synthetic Construct
gugcacaucgauucuugaccugcuuucuucuaugugaguaguguuuccggggaugugcuauacaaauaauugaagggauucuugcaguauaacuauaaauaguaaugcugcuccgggccuucagacaaaa

SEQ ID NO: 78 - RNA, 132 nt, Artificial Sequence
  Synthetic Construct
uuccaaauugaucuuuucaguccagggcagcuucccuguucugauccgggacuuugggacauuaaaaucggcugguggauucuuggacuaagaaaguauuauucauaguccuccgggaaccagccuacaaaa

SEQ ID NO: 79 - RNA, 130 nt, Artificial Sequence
  Synthetic Construct
gugcacauguaguguugaccugcuuucuucuaugugaguaguguuccuguccaugugcuauacaaauaauugaagguaguguugcaguauaacuauaaauaguaaugcugcccugucccuucagacaaaa

SEQ ID NO: 80 - RNA, 132 nt, Artificial Sequence
  Synthetic Construct
uuccaauggugguguuucaguccagggcagcuucccuguucugaccugucgaauugggacauuaaaaucggcugguguaguguuggacuaagaaaguauuauucauaguccccugucgaccagccuacaaaa

SEQ ID NO: 81 - RNA, 130 nt, Artificial Sequence
  Synthetic Construct
gugcacauuucggccugaccugcuuucuucuaugugaguaguguuucuagugaugugcuauacaaauaauugaaguucggccugcaguauaacuauaaauaguaaugcugcucuaguacuucagacaaaa

SEQ ID NO: 82 - RNA, 132 nt, Artificial Sequence
  Synthetic Construct
uuccaaauaucggccuucaguccagggcagcuucccuguucugauuuaguucuuuggaacauuaaaaucggcuggaaucggccuggacuaagaaaguauuauucauaguccuucaguguccagccuacaaaa

SEQ ID NO: 83 - RNA, 130 nt, Artificial Sequence
  Synthetic Construct
gugcacaucugguugugaccugcuuucuucuaugugaguagugucuauauugaugugcuauacaaauaauugaaggugguugugcaguauaacuauaaauaguaaugcugcuauauuucuucagacaaaa

SEQ ID NO: 84 - RNA, 132 nt, Artificial Sequence
  Synthetic Construct
uuccaaaucugguuguucaguccagggcagcuucccuguucugauauauuucuuugggacauuaaaaucggcuggucugguuguggacuaagaaaguauuauucauaguccuauguuuaccagccuacaaaa

SEQ ID NO: 85 - protein, 419 aa, Homo sapiens
MADAEVIILPKKHKKKKERKSLPEEDVAEIQHAEEFLIKPESKVAKLDTSQWPLLLKNFDKLNVRTTHYTPLACGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWKQEYVDY

SEQ ID NO: 86 - protein, 407 aa, Homo sapiens
MADAEVIILPEEDVAEIQHAEEFLIKPESKVAKLDTSQWPLLLKNFDKLNVRTTHYTPLACGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWKQEYVDYR

SEQ ID NO: 87 - protein, 387 aa, Homo sapiens
MEFLIKPESKVAKLDTSQWPLLLKNFDKLNVRTTHYTPLACGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWKQEYVDYR

SEQ ID NO: 88 - protein, 381 aa, Homo sapiens
MESKVAKLDTSQWPLLLKNFDKLNVRTTHYTPLACGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWKQEYVDYR

SEQ ID NO: 89 - RNA, 43 nt, Artificial Sequence
  Synthetic Construct
cugguugugaccugcuuucuucuaugugaguagugucuauauu

SEQ ID NO: 90 - RNA, 43 nt, Artificial Sequence
  Synthetic Construct
ugguugugcaguauaacuauaaauaguaaugcugcuauauuuc

SEQ ID NO: 91 - RNA, 44 nt, Artificial Sequence
  Synthetic Construct
cugguuguucaguccagggcagcuucccuguucugauauauuuc

SEQ ID NO: 92 - RNA, 42 nt, Artificial Sequence
  Synthetic Construct
cugguuguggacuaagaaaguauuauucauaguccuauguuu

SEQ ID NO: 93 - RNA, 44 nt, Artificial Sequence
  Synthetic Construct
cggcacgugaccugcuuucuucuaugugaguaguguucuuccca

SEQ ID NO: 94 - RNA, 41 nt, Artificial Sequence
  Synthetic Construct
cgcacgugcaguauaacuauaaauaguaaugcugccuuccu

SEQ ID NO: 95 - RNA, 44 nt, Artificial Sequence
  Synthetic Construct
cggcacguucaguccagggcagcuucccuguucugauuuccuaa

SEQ ID NO: 96 - RNA, 40 nt, Artificial Sequence
  Synthetic Construct
agcacguggacuaagaaaguauuauucauagucccuuccc

SEQ ID NO: 97 - RNA, 44 nt, Artificial Sequence
  Synthetic Construct
aagaguuugaccugcuuucuucuaugugaguaguguuuggagua

SEQ ID NO: 98 - RNA, 40 nt, Artificial Sequence
  Synthetic Construct
gaguuugcaguauaacuauaaauaguaaugcugcuggagu

SEQ ID NO: 99 - RNA, 44 nt, Artificial Sequence
  Synthetic Construct
aagaguuuacaguccagggcagcuucccuguucuguuggaguag

SEQ ID NO: 100 - RNA, 40 nt, Artificial Sequence
  Synthetic Construct
agaguuuggacuaagaaaguauuauucauaguccuggagc

SEQ ID NO: 101 - RNA, 16 nt, Artificial Sequence
  Synthetic Construct
aauguaugacaaccag

SEQ ID NO: 102 - RNA, 17 nt, Artificial Sequence
  Synthetic Construct
ggaauguaugacaacca

SEQ ID NO: 103 - RNA, 18 nt, Artificial Sequence
  Synthetic Construct
ggaauguaugacaaccag

SEQ ID NO: 104 - RNA, 17 nt, Artificial Sequence
  Synthetic Construct
gaauguaugacaaccag

SEQ ID NO: 105 - RNA, 17 nt, Artificial Sequence
  Synthetic Construct
ugggaagugacgugcug

SEQ ID NO: 106 - RNA, 14 nt, Artificial Sequence
  Synthetic Construct
gggaagugacgugc

SEQ ID NO: 107 - RNA, 18 nt, Artificial Sequence
  Synthetic Construct
uugggaagugacgugcug

SEQ ID NO: 108 - RNA, 15 nt, Artificial Sequence
  Synthetic Construct
gggaagugacgugcu

SEQ ID NO: 109 - RNA, 16 nt, Artificial Sequence
  Synthetic Construct
gcuccaugaaacucuu

SEQ ID NO: 110 - RNA, 14 nt, Artificial Sequence
  Synthetic Construct
gcuccaugaaacuc

SEQ ID NO: 111 - RNA, 15 nt, Artificial Sequence
  Synthetic Construct
cuccaugaaacucuu

SEQ ID NO: 112 - RNA, 15 nt, Artificial Sequence
  Synthetic Construct
gcuccaugaaacucu

SEQ ID NO: 113 - RNA, 44 nt, Artificial Sequence
  Synthetic Construct
ugguucuugaccugcuuucuucuaugugaguaguguugcuauac

SEQ ID NO: 114 - RNA, 41 nt, Artificial Sequence
  Synthetic Construct
aguucuugcaguauaacuauaaauaguaaugcugcgcuaua

SEQ ID NO: 115 - RNA, 45 nt, Artificial Sequence
  Synthetic Construct
uucggccugaccugcuuucuucuaugugaguaguguuucuaguga

SEQ ID NO: 116 - RNA, 42 nt, Artificial Sequence
  Synthetic Construct
uucggccugcaguauaacuauaaauaguaaugcugcucuagu

SEQ ID NO: 117 - RNA, 43 nt, Artificial Sequence
  Synthetic Construct
uaguucuuucaguccagggcagcuucccuguucugagcuacac

SEQ ID NO: 118 - RNA, 37 nt, Artificial Sequence
  Synthetic Construct
uaguucuuggguagaaaguauuauucuauucgcuaua

SEQ ID NO: 119 - RNA, 41 nt, Artificial Sequence
  Synthetic Construct
ucggccuucaguccagggcagcuucccuguucugauuuagu

SEQ ID NO: 120 - RNA, 41 nt, Artificial Sequence
  Synthetic Construct
ucggccuggacuaagaaaguauuauucauaguccuucagug

SEQ ID NO: 121 - RNA, 42 nt, Artificial Sequence
  Synthetic Construct
gauucuugaccugcuuucuucuaugugaguaguguuuccggg

SEQ ID NO: 122 - RNA, 41 nt, Artificial Sequence
  Synthetic Construct
gauucuugcaguauaacuauaaauaguaaugcugcuccggg

SEQ ID NO: 123 - RNA, 43 nt, Artificial Sequence
  Synthetic Construct
guaguguugaccugcuuucuucuaugugaguaguguuccuguc

SEQ ID NO: 124 - RNA, 41 nt, Artificial Sequence
  Synthetic Construct
uaguguugcaguauaacuauaaauaguaaugcugcccuguc

SEQ ID NO: 125 - RNA, 44 nt, Artificial Sequence
  Synthetic Construct
ugaucuuuucaguccagggcagcuucccuguucugauccgggac

SEQ ID NO: 126 - RNA, 41 nt, Artificial Sequence
  Synthetic Construct
gauucuuggacuaagaaaguauuauucauaguccuccggga

SEQ ID NO: 127 - RNA, 43 nt, Artificial Sequence
  Synthetic Construct
gugguguuucaguccagggcagcuucccuguucugaccugucg

SEQ ID NO: 128 - RNA, 42 nt, Artificial Sequence
  Synthetic Construct
guaguguuggacuaagaaaguauuauucauaguccccugucg

SEQ ID NO: 129 - RNA, 17 nt, Artificial Sequence
  Synthetic Construct
guguagcugaagaacua

SEQ ID NO: 130 - RNA, 15 nt, Artificial Sequence
  Synthetic Construct
uguagcugaagaacu

SEQ ID NO: 131 - RNA, 18 nt, Artificial Sequence
  Synthetic Construct
ucacuggaugaggccgaa

SEQ ID NO: 132 - RNA, 16 nt, Artificial Sequence
  Synthetic Construct
acuggaugaggccgaa

SEQ ID NO: 133 - skipped

SEQ ID NO: 134 - RNA, 16 nt, Artificial Sequence
  Synthetic Construct
uguagcugaagaacua

SEQ ID NO: 135 - RNA, 15 nt, Artificial Sequence
  Synthetic Construct
acuggaugaggccga

SEQ ID NO: 136 - RNA, 16 nt, Artificial Sequence
  Synthetic Construct
cacuggaugaggccga

SEQ ID NO: 137 - skipped

SEQ ID NO: 138 - RNA, 15 nt, Artificial Sequence
  Synthetic Construct
ccuggaugaaggauc

SEQ ID NO: 139 - RNA, 16 nt, Artificial Sequence
  Synthetic Construct
gacaggugaaugcugc

SEQ ID NO: 140 - RNA, 15 nt, Artificial Sequence
  Synthetic Construct
gacaggugaaugcug

SEQ ID NO: 141 - RNA, 17 nt, Artificial Sequence
  Synthetic Construct
guccuggaugaaggauc

SEQ ID NO: 142 - RNA, 16 nt, Artificial Sequence
  Synthetic Construct
uccuggaugaaggauc

SEQ ID NO: 143 - RNA, 17 nt, Artificial Sequence
  Synthetic Construct
cgacaggugaaugcugc

SEQ ID NO: 144 - skipped

SEQ ID NO: 145 - RNA, 114 nt, Artificial Sequence
  Synthetic Construct
  misc_feature 9, 38, 63, 100: n = A, U, G, or C, and is present in repeats of 4, 5, 6, 7, 8, 9, 10, 11, or 12
uuggcucunggccagcaguuugcugaagcuguuggccnggagccuaaagaauugucuuucuanuuggccauuucauaacuuuggaaauguaauggucaanagaaagaaacauga

SEQ ID NO: 146 - RNA, 115 nt, Artificial Sequence
  Synthetic Construct
  misc_feature 10, 39, 64, 101: n = A, U, G, or C, and is present in repeats of 4, 5, 6, 7, 8, 9, 10, 11, or 12
uuggcucucnggccagcaguuugcugaagcuguuggccnggagccuaaagaauugucuuucuanuuggccauuucauaacuuuggaaauguaauggucaanagaaagaaacauga

SEQ ID NO: 147 - RNA, 112 nt, Artificial Sequence
  Synthetic Construct
  misc_feature 7, 36, 61, 98: n = A, U, G, or C, and is present in repeats of 4, 5, 6, 7, 8, 9, 10, 11, or 12
uuggcunggccagcaguuugcugaagcuguuggccnggagccuaaagaauugucuuucuanuuggccauuucauaacuuuggaaauguaauggucaanagaaagaaacauga

SEQ ID NO: 148 - RNA, 115 nt, Artificial Sequence
  Synthetic Construct
  misc_feature 9, 38, 64, 101: n = A, U, G, or C, and is present in repeats of 4, 5, 6, 7, 8, 9, 10, 11, or 12
uuggcucunggccagcaguuugcugaagcuguuggccncaggagccuaaagaauugucuuucunuuggccauuucauaacuuuggaaauguaauggucaanagaaagaaacauga

SEQ ID NO: 149 - RNA, 115 nt, Artificial Sequence
  Synthetic Construct
  misc_feature 9, 38, 64, 101: n = A, U, G, or C, and is present in repeats of 4, 5, 6, 7, 8, 9, 10, 11, or 12
uuggcucunggccagcaguuugcugaagcuguuggccncaggagccuaaagaauugucguucunuuggccauuucauaacuuuggaaauguaauggucaanagaacgaaacauga

SEQ ID NO: 150 - RNA, 116 nt, Artificial Sequence
  Synthetic Construct
  misc_feature 9, 38, 65, 102: n = A, U, G, or C, and is present in repeats of 4, 5, 6, 7, 8, 9, 10, 11, or 12
uuggcucunggccagcaguuugcugaagcuguuggccncaggagccuaaagaauugucuuucuanguggccauuucauaacuuuggaaauguaauggucacnagaaagaaacauga

SEQ ID NO: 151 - RNA, 137 nt, Artificial Sequence
  Synthetic Construct
uuggcucuuguaagaggccagcaguuugcugaagcuguuggccguacucaggagccuaaagaauugucuuucuaugaucccuuggccauuucauaacuuuggaaauguaauggucaauccgguagaaagaaacauga

SEQ ID NO: 152 - RNA, 137 nt, Artificial Sequence
  Synthetic Construct
uuggcucuugaucccggccagcaguuugcugaagcuguuggccuccggcaggagccuaaagaauugucuuucuauguaggauuggccauuucauaacuuuggaaauguaauggucaagugaguagaaagaaacauga

SEQ ID NO: 153 - RNA, 138 nt, Artificial Sequence
  Synthetic Construct
uuggcucucugaucccggccagcaguuugcugaagcuguuggccuccggcaggagccuaaagaauugucuuucuaugaucccuuggccauuucauaacuuuggaaauguaauggucaauccgguagaaagaaacauga

SEQ ID NO: 154 - RNA, 137 nt, Artificial Sequence
  Synthetic Construct
uuggcugcugaucccggccagcaguuugcugaagcuguuggccuccggcaggagccuaaagaauugucuuucuaugaucccuuggccauuucauaacuuuggaaauguaauggucaauccgguagaaagaaacauga

SEQ ID NO: 155 - skipped

SEQ ID NO: 156 - RNA, 137 nt, Artificial Sequence
  Synthetic Construct
uuggcucuugaucccggccagcaguuugcugaagcuguuggccuccggugggagccuaaagaauugucuuucuaugaucccuuggccauuucauaacuuuggaaauguaauggucaauccgguagaaagaaacauga

SEQ ID NO: 157 - RNA, 138 nt, Artificial Sequence
  Synthetic Construct
uuggcucucugaucccggccagcaguuugcugaagcuguuggccuccggugggagccuaaagaauugucuuucuaugaucccuuggccauuucauaacuuuggaaauguaauggucaauccgguagaaagaaacauga

SEQ ID NO: 158 - RNA, 137 nt, Artificial Sequence
  Synthetic Construct
uuggcugcugaucccggccagcaguuugcugaagcuguuggccuccggugggagccuaaagaauugucuuucuaugaucccuuggccauuucauaacuuuggaaauguaauggucaauccgguagaaagaaacauga

SEQ ID NO: 159 - RNA, 136 nt, Artificial Sequence
  Synthetic Construct
uuggcucuugaucccggccagcaguuugcugaagcuguuggccuccggcaggagccuaaagaauugucuuucuugaucccuuggccauuucauaacuuuggaaauguaauggucaauccgguagaaagaaacauga

SEQ ID NO: 160 - RNA, 136 nt, Artificial Sequence
  Synthetic Construct
uuggcucuugaucccggccagcaguuugcugaagcuguuggccuccggcaggagccuaaagaauugucguucuugaucccuuggccauuucauaacuuuggaaauguaauggucaauccgguagaacgaaacauga

SEQ ID NO: 161-169 - skipped (9 entries)

SEQ ID NO: 170 - RNA, 137 nt, Artificial Sequence
  Synthetic Construct
uuggcucuugaucccggccagcaguuugcugaagcuguuggccuccggcaggagccuaaagaauugucuuucuaugaucccguggccauuucauaacuuuggaaauguaauggucacuccgguagaaagaaacauga

SEQ ID NO: 171 - RNA, 130 nt, Artificial Sequence
  Synthetic Construct
gugcacaucugauccugaccugcuuucuucuaugugaguaguguuuccggugaugugcuauacaaauaauugaaggcgauccugcaguauaacuauaaauaguaaugcugcuccgguccuucagacaaaa

SEQ ID NO: 172 - RNA, 164 nt, Artificial Sequence
  Synthetic Construct
gugcacaucugauccugaccugcuuucuucuaugugaguaguguuuccggugaugugcuauacaaauaauugaaggcgauccugcaguauaacuauaaauaguaaugcugcuccgguccuucagacaaaaucuaucuaucuagagcggacuucgguccgcuuuu

SEQ ID NO: 173 - RNA, 183 nt, Artificial Sequence
  Synthetic Construct
gugcucgcuucggcagcacauauacuagugcacaucugauccugaccugcuuucuucuaugugaguaguguuuccggugaugugcuauacaaauaauugaaggcgauccugcaguauaacuauaaauaguaaugcugcuccgguccuucagacaaaaucuagagcggacuucgguccgcuuuu

SEQ ID NO: 174 - RNA, 65 nt, Artificial Sequence
  Synthetic Construct
gugcacaucugauccugaccugcuuucuucuaugugaguaguguuuccggugaugugcuauacaa

SEQ ID NO: 175 - RNA, 65 nt, Artificial Sequence
  Synthetic Construct
ggaauugaaggcgauccugcaguauaacuauaaauaguaaugcugcuccgguccuucagacaaaa

SEQ ID NO: 176 - skipped

SEQ ID NO: 177 - RNA, 139 nt, Artificial Sequence
  Synthetic Construct
  misc_feature 9, 39, 63, 92: n = A, U, G, or C, and is present in repeats of 4, 5, 6, 7, 8, 9, 10, 11, or 12
gugcacaungaccugcuuucuucuaugugaguaguguungugcuauacaaauaauugaaggcngcaguauaacuauaaauaguaaugcugcnccuucagacaaaaucuaucuaucuagagcggacuucgguccgcuuuu

SEQ ID NO: 178 - RNA, 158 nt, Artificial Sequence
  Synthetic Construct
  misc_feature 36, 66, 90, 119: n = A, U, G, or C, and is present in repeats of 4, 5, 6, 7, 8, 9, 10, 11, or 12
gugcucgcuucggcagcacauauacuagugcacaungaccugcuuucuucuaugugaguaguguungugcuauacaaauaauugaaggcngcaguauaacuauaaauaguaaugcugcnccuucagacaaaaucuagagcggacuucgguccgcuuuu

SEQ ID NO: 179 - RNA, 50 nt, Artificial Sequence
  Synthetic Construct
  misc_feature 9, 39: n = A, U, G, or C, and is present in repeats of 4, 5, 6, 7, 8, 9, 10, 11, or 12
gugcacaungaccugcuuucuucuaugugaguaguguungugcuauacaa

SEQ ID NO: 180 - RNA, 55 nt, Artificial Sequence
  Synthetic Construct
  misc_feature 13, 42: n = A, U, G, or C, and is present in repeats of 4, 5, 6, 7, 8, 9, 10, 11, or 12
ggaauugaaggcngcaguauaacuauaaauaguaaugcugcnccuucagacaaaa

SEQ ID NO: 181 - RNA, 130 nt, Artificial Sequence
  Synthetic Construct
gugcacauccauuagugcccugcuuucuucuaugugaguagugguggucucgaugugcuauacaaauaauugaaggcauuaguggaguauaacuauaaauaguaaugcuuuggucucccuucagacaaaa

SEQ ID NO: 182 - RNA, 132 nt, Artificial Sequence
  Synthetic Construct
uuccagguccauuagugcgguccagggcagcuucccuguucuguggucucauccugggacauuaaaaucggcugguccauugguugacuaagaaaguauuauucauaguugggucucaaccagccuacaaaa

SEQ ID NO: 183 - RNA, 130 nt, Artificial Sequence
  Synthetic Construct
gugcacaucgaguagcgaccugcuuucuucuaugugaguaguguuuccugagaugugcuauacaaauaauugaagcgaguagcgcaguauaacuauaaauaguaaugcugcuccugagcuucagacaaaa

SEQ ID NO: 184 - RNA, 133 nt, Artificial Sequence
  Synthetic Construct
uuccaaagugaguagcucaguccagggcagcuucccuguucugauccugaaauuugggacauuaaaaucggcugguugaguagcggacuaagaaaguauuauucauaguccuccugaaaaccagccuacaaaa

SEQ ID NO: 185 - RNA, 130 nt, Artificial Sequence
  Synthetic Construct
gugcacaucuagauaugaccugcuuucuucuaugugaguaguguuuaaaaugaugugcuauacaaauaauugaagcuagauaugcaguauaacuauaaauaguaaugcugcuaaaaugcuucagacaaaa

SEQ ID NO: 186 - RNA, 133 nt, Artificial Sequence
  Synthetic Construct
uuccaaagguagauauucaguccagggcagcuucccuguucugauaaaaggauuugggacauuaaaaucggcugguguagauauggacuaagaaaguauuauucauaguccuaaaaggaaccagccuacaaaa

SEQ ID NO: 187 - RNA, 130 nt, Artificial Sequence
  Synthetic Construct
gugcacaucuauccuugaccugcuuucuucuaugugaguaguguuugcugcgaugugcuauacaaauaauugaagcuauccuugcaguauaacuauaaauaguaaugcugcugcugcgcuucagacaaaa

SEQ ID NO: 188 - RNA, 133 nt, Artificial Sequence
  Synthetic Construct
uuccaaagauauccuuucaguccagggcagcuucccuguucugaugcugcaguuugggacauuaaaaucggcugguguauccuuggacuaagaaaguauuauucauaguccugcugcagaccagccuacaaaa

SEQ ID NO: 189 - RNA, 130 nt, Artificial Sequence
  Synthetic Construct
gugcacauccuugugcgaccugcuuucuucuaugugaguagugucuggguugaugugcuauacaaauaauugaaggcuugugcgcaguauaacuauaaauaguaaugcugcuggguuccuucagacaaaa

SEQ ID NO: 190 - RNA, 132 nt, Artificial Sequence
  Synthetic Construct
uuccagauccuugugcugaguccagggcagcuucccuguucucauggguaacucugggacauuaaaaucggcugguguuugugcggacuaagaaaguauuauucauaguccugggugaaccagccuacaaaa

SEQ ID NO: 191 - RNA, 131 nt, Artificial Sequence
  Synthetic Construct
gugcacaugguuacaugaccugcuuucuucuaugugaguaguguugaggagauaugugcuauacaaauaauugaagguuuacaugcaguauaacuauaaauaguaaugcugugaggguccuucagacaaaa

SEQ ID NO: 192 - RNA, 132 nt, Artificial Sequence
  Synthetic Construct
uuccaaagacuuacaugcaguccagggcagcuucccuguucugugagggcgauuugggacauuaaaaucggcugguguuuacauggacuaagaaaguauuauucauagucugagggaaaccagccuacaaaa

SEQ ID NO: 193 - DNA, 30 bp, Artificial Sequence
  Synthetic Construct
gcctatatcaccggataaggatcagcccca

SEQ ID NO: 194 - DNA, 30 bp, Artificial Sequence
  Synthetic Construct
gcctatatcaccggatagggatcagcccca

SEQ ID NO: 195 - DNA, 30 bp, Artificial Sequence
  Synthetic Construct
gcctatatcaccggatcaggatcagcccca

SEQ ID NO: 196 - DNA, 15 bp, Artificial Sequence
  Synthetic Construct
ggatagggatcagcc

SEQ ID NO: 197 - DNA, 15 bp, Artificial Sequence
  Synthetic Construct
ggaggtggatcagcc